(19) United States
(12) Patent Application Publication
(10) Pub. No.: US 2017/0238401 A1
(43) Pub. Date: Aug. 17, 2017

(54) Solid State Lighting Systems

(71) Applicant: INNOSYS, INC., Salt Lake City, UT (US)

(72) Inventors: Laurence P. Sadwick, Salt Lake City, UT (US); Skylar Stoddard, Salt Lake City, UT (US); Derrick K. Kress, Salt Lake City, UT (US)

(21) Appl. No.: 15/114,064

(22) PCT Filed: Jan. 26, 2015
(86) PCT No.: PCT/US15/12965

§ 371 Date: Jul. 25, 2016

(51) Int. Cl. H05B 37/02; A61N 5/01; F21K 9/235; A61N 5/06; H05B 33/08; F21V 21/15

(52) U.S. Cl. CPC H05B 37/0281; H05B 33/0896; H05B 33/0857; H05B 37/0272; F21V 21/15; F21K 9/235; A61N 5/0618; A61N 5/01; F21K 9/27

(57) ABSTRACT

A solid state lighting system performing color changes based on time of day. The light source may be an OLED panel, a quantum dot, pin point source. The light may be programed based on circadian rhythms, and may have separate controllers for each light source. T light may be a threaded lamp, or be installed into a fluorescent lamp fixture.

### BACKGROUND

A major source of wasted and excessive energy usage is inefficient lighting such as incandescent bulbs and older types of ballasts used for T8 and T12 fluorescent lights. Interest has grown rapidly in replacing incandescent lights with more efficient lighting, such as fluorescent lighting and light emitting diodes (LEDs). However, great room for improvement remains in efficient lighting.

### SUMMARY

Some embodiments of the present invention provide for efficient, controllable lighting, including task lighting with smart control and reporting features.

Some embodiments of the present invention provide for energy collection, storage and/or usage from solar cells or collectors embedded in or mounted on curtains, drapes, shutters, shades, blinds or other structures adapted to control sunlight through a window or incident on another area such as a patio or porch.

A fixed and/or portable single or array of solar energy conversion cells or units such a photovoltaic energy converters that are incorporated into, for example but not limited to, shades, curtains, drapes, shutters, blinds, etc. that are intended to partially or completely block or screen sunlight, solar energy and/or other sources of light/energy is disclosed herein for wirelessly controlling one or more lights or other devices. An embodiment of the control panel includes a solar panel, a regulator connected to the solar panel, a power storage device connected to the regulator, a wireless transceiver, a controller connected to the power storage device, and a user interface connected to the controller. The user interface is adapted to accept control input and provide it to the controller. The controller is adapted to transmit commands on the wireless transceiver.

In an embodiment of the control panel, the user interface comprises a lighting control interface.

In an embodiment of the control panel, the lighting control interface comprises a dimming interface.

In an embodiment of the control panel, the lighting control interface comprises a multi-color lighting control interface.

An embodiment of the control panel also includes a display, and the controller is adapted to display lighting status on the display.

An embodiment of the control panel also includes a light sensor, and the controller is adapted to generate lighting control commands at least in part based on an ambient light level measured by the light sensor.

In an embodiment of the control panel, the user interface comprises a temperature control interface.

An embodiment of the control panel also includes a temperature sensor, and the controller is adapted as an HVAC controller to read an ambient temperature from the temperature sensor and to transmit the ambient temperature.

In an embodiment of the control panel, the controller is adapted to transmit temperature settings commands.

In an embodiment of the control panel, the controller is adapted to take priority as a master HVAC controller in a group of control panels with temperature sensors.

In an embodiment of the control panel, the user interface includes a touch sensitive display screen and a graphical user interface.

In an embodiment of the control panel, the controller is adapted to store customized settings.

In an embodiment of the control panel, the controller is adapted to store multiple user preferences.

In an embodiment of the control panel, the controller is programmable to add additional devices which can be controlled by the control panel.

In an embodiment of the control panel, the controller is adapted to receive a notice of error conditions in a remote device and to transmit a user alert of the error conditions.

In some embodiments of the control panel, the controller contains USB, barrel plugs, and other connectors with which consumer electronics or rechargeable batteries may be recharged or interfaced.

An embodiment of the control panel also includes a display, and the controller is adapted to receive and display information from a remote device on the display such as voltage, current, power, phase, watt hours, power factor, VA, and lead-lag.

In an embodiment of the control panel, the controller is adapted to receive electricity rates and to customize the commands based on the electricity rates to reduce electricity costs.

In an embodiment of the control panel, the solar panel may be angled to maximize light reception.

In an embodiment of the control panel, the user interface is detachable.

Another embodiment of a control panel includes a solar panel, a regulator connected to the solar panel, a power storage device connected to the regulator, a wireless transceiver, a controller connected to the power storage device, a temperature sensor connected to the controller, a light sensor connected to the controller, and a user interface connected to the controller. The user interface is adapted to accept multi-color dimming light control input. The controller is adapted to generate light control commands based in part on the user interface and in part on an ambient light level measured by the light sensor. The user interface is also adapted to accept temperature control input. The controller is also adapted to generate temperature control commands based in part on the user interface and in part on an ambient temperature measured by the temperature sensor and on a remote ambient temperature measured by a remote control panel, and to transmit the light control commands and the temperature control commands on the wireless transceiver.

In another embodiment of the present invention, control and or monitor signals are sent to an additional unit that is connected to the power lines and the commands sent from the present solar powered invention are transmitted via the power lines to the intended device to be controlled. In a similar fashion, monitoring information can be sent to and from the present solar powered remote transceiver invention via the power lines.

The present invention can be used with a holster that provides additional solar power to power and charge up the remote. Such a holster can be designed to be both attractive and decorative while providing power to the remote unit. Such a holster can also have the appearance of a conventional “wall” dimmer or light control. and can be used to control fan(s), portable air conditioner(s), window air conditioner(s), evaporative cooler,(s), etc., combinations of these and other types heating, cooling, flow, HVAC, registers, vents, ducts, etc.

These devices can be controlled by the user through a smart phone, tablet, personal device, computers, etc., running software applications while connected to a home or business wifi network. This network can be connected to a server or main control unit that communicates to the remote devices wirelessly.

It is also possible to connect the server to the remote devices via the power lines that already exist in, for example, the home or business, etc.

These devices can also be controlled via Bluetooth connection through a smart phone, tablet, personal device, computers, etc. In some embodiments of the present invention, this method typically bypasses the wifi network and the server or main controller and connects directly to the remote devices wirelessly. In other embodiments more than one of the methodologies discussed herein may be used in various implementations.

This system can be used in all control devices such as heaters, air-conditioners, televisions, personal fans, air purifiers, DVD units, DVR, satellite, cable boxes, etc., using an infrared LED that can output commands the devices recognize. The remote device unit can also capture infrared commands from remote controls that operate their respected devices.

Using Bluetooth to connect to the remote devices, the devices can adjust appliances and/or devices in a room, home, or business or any other locations according to the user's desires. When the user enters a room, for example, home, or business the devices can recognize the Bluetooth device of the user and adjust the lighting, heating, air-conditioning, blinds, including solar blinds, solar drapes, solar shutters, solar curtains, solar shades, etc. and other devices.

These remote devices can relay data and information back to the user on their smart phone, tablet, personal device, computers, laptops, servers, cloud, etc., such as battery charge, voltage, current usage, power, state, etc.

This system can be used with speakers that may be built into the control panel, or into the lights themselves in any form factor to provide music and other audio signals for a variety of purposes including but not limited to receiving audio signals and information including music that are sent to them wirelessly.

The user can create scheduled remote events, tasks, etc., for example, for infrared controlled appliances or devices to carry out specific functions and at specific times with simple to complex sequences that can also include scheduling which can optionally be conditionally modified.

This system can also be used with sensors that sense water, moisture, barometric pressure and humidity and can be used for irrigation, flood prevention, humidity control, etc.

This system can also be used for motion sensing and proximity sensing to aid in home or business security and for sensing when an individual enters a room or building and customizing the infrared controlled devices based on the individual, other information including but not limited to time, day of the week and/or month, date, user input, weather conditions, other input and feedback, etc.

A standard, custom or adapted remote can be used with this system to aid individuals without access to smart phones, tablets, computers, etc., so they can still control and interact with their infrared devices remotely. Such a remote can also be wired and wireless and can use existing visual devices such as televisions, computer and other monitors, etc. Embodiments of the present invention can also use microphones to receive and execute voice commands including using voice recognition as well as speakers to provide information and query as well as provide alerts and warnings. Other embodiments can use gesturing including hand gesturing as well as combinations of other methods herein to create, sort, sequence, schedule, organize, evaluate, make decisions, execute commands and perform tasks, implement and act on conditional statements, stack, analyze, etc. In many cases, the scheduling and sequencing as well as the organization and sorting and even the creation can be conditional on the results and outcomes of previous events, scheduled tasks, information, input, both internal and external information and stimuli, etc. The present invention allows overriding previously scheduled, sequenced and/or sorted operations and executions to adapt to new stimuli, input, results, information, etc. or to ignore such and continue with the program sequencing and schedule or to allow a combination of both or to allow weighted combinations of both or other scenarios as well including adaptable responses and requests to changes in scenarios, situations, results, input, unexpected outcomes and results, etc. Some of these responses include interacting with the user or others such as neighbors, family and friends, guardians, surveillance personnel, etc.

These remote devices can relay data and information back to the user on their smart phone, tablet, personal device, computers, etc., such as battery charge, voltage, current usage, power, state, etc.

This invention contains lighting devices that can be controlled wirelessly by the same convention, signals, protocols, etc. as the remote devices. These lighting devices, for example, can be dimmed, turned on/off, turned, rotated, moved, etc., from a smart phone, tablet, personal device, etc. The remote movement works, for example, by way of servos in the arms and/or body of the lamp, light, light fixture, ballast, desk lamp, etc. In some embodiments of the present invention, The lighting devices, if capable of changing colors, can be controlled to change color based on the users wants.

These lighting devices can be powered by a battery that is charged by solar panels installed into shades/blinds/shutters/curtains/drapes or, for example, into the lighting devices themselves. The lighting can be either or both inside and/or outside lights that can be set, programmed, controlled, etc. to time shift and light (i.e., time of day such that it is sunny to time of night when it is dark) shifted by for example using storage devices including but not limited to batteries, fuel cells, etc.

The shades/blinds/shutters/drapes/curtains can interact with the lighting devices as in general they can are part of the same network.

The lights can be set-up to a schedule and for example sequence including but not limited to pre-programmed input, etc. based on minutes, hours, days, months, years. They can be programmed to carry out commands at specific times set by the user. For example, The light can be programmed to turn on in the morning in a diverse number of ways from gently and slowly to abrupt, assertively turning on to awake the user.

Combined with motion sensors the lighting devices can be used for security purposes and/or for providing light in the dark when motion is sensed.

The lighting devices can respond to user's presets when a familiar Bluetooth device is recognized and in range. This can consist of but is not limited to, dimming level, position, color, or status, temperature, rates, other parameters/conditions, etc.

The lighting device(s) can dim either all or the respective brightness or can prioritize which lighting device(s) should continue to use and receive certain power level(s) when the supplied battery power from the shades/blinds/drapes/curtains/shutters is/are getting low in order to conserve battery power.

This summary provides only a general outline of some embodiments according to the present invention. Many other embodiments of the present invention will become more fully apparent from the following detailed description.

### BRIEF DESCRIPTION OF THE DRAWINGS

A further understanding of the various exemplary embodiments may be realized by reference to the figures which are described in remaining portions of the specification. In the figures, like reference numerals may be used throughout several drawings to refer to similar components.

FIG. 1 depicts a window with closed vertical blinds with solar collectors in accordance with some embodiments of the invention.

FIG. 2 depicts a window with open solar collecting blinds, shutters, drapes, or curtains, etc. in accordance with some embodiments of the invention.

FIG. 3 depicts a window with open vertical blinds with solar collectors in accordance with some embodiments of the invention.

FIG. 4 depicts a window with closed horizontal blinds with solar collectors in accordance with some embodiments of the invention.

FIG. 5 depicts a window with open horizontal blinds with solar collectors in accordance with some embodiments of the invention.

FIG. 6 depicts a window with open horizontal blinds with solar collectors in accordance with some embodiments of the invention.

FIG. 7 depicts a window with strings of solar cells forming a window covering in accordance with some embodiments of the invention.

FIG. 8 depicts a window with strings of solar cells forming a window covering with another connection mechanism in accordance with some embodiments of the invention.

FIG. 9 depicts a block diagram of a home automation system with mobile sensors in accordance with some embodiments of the invention.

FIG. 10 depicts a block diagram of wireless/wired connections between components of a home automation system with mobile sensors in accordance with some embodiments of the invention.

FIGS. 11-13 depict perspective, top, and side views of an IR sensor and/or transmitter suitable for use in some embodiments of a home automation system in accordance with some embodiments of the invention.

FIGS. 14-16 depicts side, perspective and top views of another IR sensor and/or transmitter suitable for use in some embodiments of a home automation system in accordance with some embodiments of the invention.

FIG. 17 depicts example usage of a home automation system with mobile sensors in a home floorplan in accordance with some embodiments of the invention.

FIG. 18 depicts a diagram of WiFi connections in an example embodiment of a home automation system in accordance with some embodiments of the invention.

FIG. 19 depicts a diagram of Bluetooth connections in an example embodiment of a home automation system in accordance with some embodiments of the invention.

FIG. 20 is a perspective view of a plantation shutter window covering with solar collection louvers in accordance with some embodiments of the invention.

FIG. 21 is a front view of a plantation shutter window covering with solar collection louvers in accordance with some embodiments of the invention.

FIG. 22 is a front view of a solar collection louver for a plantation shutter window covering in accordance with some embodiments of the invention.

FIG. 23 is a perspective top view of an automated register assembly suitable for use in some embodiments of a home automation system in accordance with some embodiments of the invention.

FIG. 24 is a top view of an automated register assembly suitable for use in some embodiments of a home automation system in accordance with some embodiments of the invention.

FIG. 25 is a perspective bottom view of an automated register assembly suitable for use in some embodiments of a home automation system in accordance with some embodiments of the invention.

FIG. 26 is a bottom view of an automated register assembly with substantially open vents in accordance with some embodiments of the invention.

FIG. 27 is a bottom view of an automated register assembly with substantially closed vents in accordance with some embodiments of the invention.

FIGS. 28-29 are perspective front views of another automated register assembly with a motorized directional control in open and closed positions in accordance with some embodiments of the invention.

FIGS. 30-31 are side views of the automated register assembly of FIGS. 28-29 in open and closed positions in accordance with some embodiments of the invention.

FIGS. 32-33 are perspective front views of another automated register assembly with a motorized vent control in open and closed positions in accordance with some embodiments of the invention.

FIG. 34 is a side view of the register assembly of FIGS. 32-33.

FIGS. 35-36 are perspective bottom views of the register assembly of FIGS. 32-33 with a motorized vent control in open and closed positions in accordance with some embodiments of the invention.

FIGS. 37-38 are bottom views of the register assembly of FIGS. 32-33 with a motorized vent control in open and closed positions in accordance with some embodiments of the invention.

FIG. 39 is a block diagram of wireless monitoring of power conversion and usage for collection and storage of power from solar window coverings in accordance with some embodiments of the invention.

FIGS. 40-42 depict an IR interpreter in side, front perspective and rear perspective views in accordance with some embodiments of the invention.

FIGS. 43-44 depict an articulating desk lamp with one or more rotating solid state lighting panels in accordance with some embodiments of the invention.

FIGS. 45-46 depict a motorized articulating desk lamp with one or more rotating solid state lighting panels in accordance with some embodiments of the invention.

FIGS. 47-48 depict a lamp base fitted with a solid state lighting panel that can be rotated in accordance with some embodiments of the invention.

FIGS. 49-50 depict a lamp base fitted with multiple solid state lighting panels that can be rotated in accordance with some embodiments of the invention.

FIG. 51 depicts a lamp base fitted with four rectangular solid state lighting panels in accordance with some embodiments of the invention.

FIG. 52 depicts a lamp base fitted with one or more solid state lighting panels and/or LEDs or QDs or other substantially point light sources under a cylindrical cover in accordance with some embodiments of the invention.

FIG. 53 depicts a lamp base fitted with a solid state lighting panel and a LED or QD or other substantially point light source in accordance with some embodiments of the invention.

FIG. 54 depicts a lamp base fitted with solid state lighting panel and a LED or QD or other substantially point light source in accordance with some embodiments of the invention.

FIGS. 55-58 are circuit diagrams of power supply/dimming control circuits for solid state lighting devices such as LED, OLED, QD, etc. in accordance with some embodiments of the invention.

FIG. 59 is a block diagram of a power supply/dimming control circuit with selectable linear and switching regulation for solid state lighting devices such as LED, OLED, QD, etc. in accordance with some embodiments of the invention.

FIG. 60 is a block diagram of a power supply/dimming control circuit for solid state lighting devices such as LED, OLED, QD, etc. in accordance with some embodiments of the invention.

FIG. 61 is a block diagram of a solid state lighting power supply/dimming circuit in accordance with some embodiments of the invention.

FIG. 62 is a schematic illustration of an example of a common cathode for, for example, a three color OLED stack in accordance with some embodiments of the invention.

FIG. 63 is a schematic illustration of an example of a common anode for, for example, a three color OLED stack in accordance with some embodiments of the invention.

FIG. 64 is a schematic illustration of 3 channel (i.e. RGB or RYB) common cathode solid state lights with driver regulator for each channel in accordance with some embodiments of the invention.

FIG. 65 is a schematic illustration of 3 channel (i.e. RGB or RYB) common anode solid state lights with driver regulator for each channel in accordance with some embodiments of the invention.

FIG. 66 is a simplified schematic illustration of 4 channel solid state lights with current control for each channel in accordance with some embodiments of the invention.

FIG. 67 is a simplified schematic illustration of 4 channel solid state lights with shared current control for each channel in accordance with some embodiments of the invention.

FIG. 68 is a simplified schematic illustration of 4 channel solid state lights with shared current control in accordance with some embodiments of the invention.

FIG. 69 is a block diagram of a power supply/dimming control circuit for a solid state lighting device that allows both manual/local dimming which can be selectively allowed or overridden by a wired and/or wireless interface in accordance with some embodiments of the invention.

FIG. 70 is a block diagram of an example AC to low voltage DC output bus for a solid state lighting system in accordance with some embodiments of the invention.

FIG. 71 is a block diagram of another example AC to low voltage DC output bus for a solid state lighting system in accordance with some embodiments of the invention.

FIG. 72 is a block diagram of a solid state lighting system including an example N-channel driver for controlling/monitoring multiple individual OLED panels, stacked OLED panels, RGB/RYB etc., OLED panels in accordance with some embodiments of the invention.

FIG. 73 is a block diagram of a wireless controller/monitor for a solid state lighting system in accordance with some embodiments of the invention.

FIG. 74 is a block diagram illustrating example wired and wireless control input types and sources that can be fed to the wireless controller or to solid state lighting drivers directly in accordance with some embodiments of the invention.

FIG. 75 is a simplified schematic illustration of 3 channel solid state lights with floating output current in accordance with some embodiments of the invention.

FIG. 76 depicts a retrofit of a fluorescent lighting fixture with an array of solid state lighting panels in accordance with some embodiments of the invention.

FIG. 77 depicts a retrofit of a fluorescent lighting fixture with a solid state lighting panel in accordance with some embodiments of the invention.

### DESCRIPTION

Various embodiments of the present invention provide efficient lighting systems, including smart task lighting that can be controlled locally and/or remotely and which provides monitoring or reporting features. In some embodiments, panel lighting and point source lighting including, but not limited to, light emitting devices (LEDs), organic light emitting diodes (OLEDs) and phosphorescent organic light emitting diodes (PHOLEDs), quantum dots (QDs), other solid state lighting (SSL), other type of lighting and lamps, etc. and/or combinations of these, etc. provide high efficiency lighting for a variety of applications.

In some embodiments, task lights are mounted on articulated and/or telescoping arms. The light source can swivel in any direction, including horizontally or vertically, and can be provided with motorized control in one or more locations, such as at the mounting point between the light source and a support arm, or at the base of a support arm, or at one or more joints of an articulated support arm.

Various embodiments of the smart task light provide one or more of the following features:

Remote control of motorized aiming including wired and wireless (i.e., powerline control, RS232, USB, SPI, SPC, I2C, etc., WiFi, Bluetooth, ZigBee, IEEE 801, ISM, infrared (IR), etc.) so as to move the light source up and down, left and right, more or additional axes of motion/rotation, etc.

The present invention can be dimmed and turned on/off remotely. The present invention can be tilted/aimed/pointed/flipped/closed/etc. remotely. The present invention can be color changing (i.e., include RGB) in addition to various colors of white, color temperatures of white, full spectrum lighting, etc. Embodiments of the present invention can use RGB color changing plus white light (i.e., WRGB) and/or RGB color changing plus amber light (i.e., RGBA) and/or RGB color changing plus white and/or amber light (WRGBA), etc., including combinations of LED, OLED, QD, other SSL, other lighting, etc. and/or combinations of these, etc.

The present invention can be controlled by smart phones (i.e., iPhones, Androids, Samsung), tablets (iPads, iPods, Androids, Kindle, Samsung, etc.), laptops, desk top computers, etc.

The present invention can have integrated built-in battery back-up/storage.

The present invention can be used as an emergency, camping, personal or portable light and can be used as an emergency beacon.

The present invention can respond to/interact with RFID and other such signals and systems

The present invention can be solar power and/or solar charged.

The present invention can be used as an alarm clock in numerous modes including an embodiment where the light comes on gradually and increases in intensity while, for example, rotating from a horizontal facing down light source direction to either a vertical light facing direction or a horizontal light facing up direction or alternate between various facing directions while also providing optional sound (words, alarms, music, etc.).

The present invention can be voice activated and controlled.

The present invention can provide monitoring including input and output current, voltage, power, etc. (analytics) and also respond to motion, sound, light, etc. and report and store any or all monitoring information, conditions, etc.

The present invention can provide color changing remotely and also be sound activated including changing colors to sound, music, temperature, vibration, etc.

The present invention can be implemented to track sound, motion, light, vibrations, etc.

The present invention can be, but not limited to, a desk lamp, a track lamp, a task lamp, a table lamp, a floor lamp, a room lamp, a downlight, a can light, sconce, pendant, etc.

The present invention can be programmed to turn on or off by time of day, day of week, event-based including dawn or dusk, etc. The present invention can use motion sensors that can do, for example, multiple duties—turning on/off lights, alerting that there are people there, heating or cooling spaces, being part of a burglar alarm, etc.

The present invention can track, report, store, display, show, log, control, manage, control, monitor, respond, feedback, distribute, modify, interact, allocate, respond, adapt, the position and angle, etc. either dynamically or statically or both of the lamp, including of the motors, actuators, light, power, and related items, etc.

The present invention can use sensors of any type including but not limited to position, acceleration, velocity, angular, height, incline, decline, slope, color temperature, light, pressure, touch, mechanical, vibration, strain, stress, etc.

The present invention can use storage of lighting direction, to remember previous settings to repeat again and again and to also learn and store new ones; to store favorites; to make new favorites; etc.

The present invention including lamp embodiments can sway and move including in arbitrary directions to various types of stimuli including, but not limited to, sounds, music, noise, vibrations, light, movement, pre-programming, user-programming, remote programming, etc.

Voice commands, sound control, color sensors, microphones, tones, audio, volume level, etc. can be used with the present invention.

The present invention can use solar conversion to store energy to turn on later.

The present invention can be, but is not limited to, a task lamp, desk lamp, a wall lamp, a can lamp a ceiling lamp, a track lamp, a lamp fixture, a sconce lamp, a pendant lamp, an accent lamp, under counter lamps, over counter lamps, cabinet lamps, part of a multi-lamp fixture, part of a fan lamp, a bed lamp, a reading lamp, a floor lamp, a bed headboard lamp, a bed footboard lamp, a table lamp, a multi-purpose lamp, a bathroom lamp, a vanity lamp, a kitchen lamp, a minor lamp, a picture lamp, a dresser lamp, a bathroom lamp, a closet lamp, a bath lamp, a shower lamp, combinations of these, multiples of these, etc.

The present invention offers healthy, economical, energy-efficiency benefits. With the energy savings and the potentially energy-neutral nature of the present invention there are both economical and human health benefits associated with adopting the present invention.

The present invention also may improve human health when used in certain circumstances such as light therapy, in hospitals including children hospitals, critical care, intensive care, neonatal, maternity, short term and long term care, and psychiatric hospitals, schools, office environments and buildings to alleviate anxiety and tension with soothing color tones, choices and intensities, as a wake-up aid to naturally wake due to an increase in light exposure of appropriate wavelengths, and in other capacities such as streetlights and street signs where different colors/tones/amplitudes/hues, etc. of light may be beneficial.

The present invention may include lights such as LEDs, OLEDs, QDs, fluorescent lighting and even, in certain cases, incandescent bulbs, etc. on the IR modules and may also employ solar cells to assist in supplying power and charging, or to fully power the device. Power from the solar cells may also be applied back to the grid to supply power/energy elsewhere or to be used throughout the home or building to power other devices or to be provided back to the electrical grid. In some embodiments of the present invention batteries may also be incorporated into the lighting. The present invention can provide full spectrum or selected user or programmed partial spectrum lighting, for example but not limited to, that changes predominant wavelengths/colors depending on the time of day or night and can be dimmed up, for example, in the morning and dimmed down at night and bedtime. Such lighting can be used for producing increased health, immunity to diseases, productivity, learning and focus, and other health benefits for hospitals, schools, libraries, convalescent homes, assisted living, colleges and universities, dormitories, office and other buildings of all kinds, etc.

The present invention may be used to provide emergency lighting in hospitals, schools, libraries, convalescent homes, assisted living, colleges and universities, dormitories and buildings of all kinds. The invention may also be used as an emergency beacon where lights and sounds may take place when one or more of any number of, for example, disasters or emergencies occur such as fires, earthquakes, tornadoes, floods, and any other event when an alarm is needed. The present invention also may receive signals from the emergency broadcast systems and radio weather stations and other sources to further display information about current emergency conditions. Units may communicate to other units in the nearby geographical area to alert of any current danger or emergency situation. The present invention may also include sensors such as those used to detect temperature, smoke, CO, propane, natural gas, and other airborne particles/chemicals to further provide safe environment monitoring in any situation.

To ensure that the IR transmitter or IR transmitter array is visible to any and all devices in the current area, the IR unit may employ gimbals, servo motors, stepper motors, linear motors and any type of IR lens such as Fresnel, convex, concave, aspheric, achromatic, ball, half-ball, plano-convex and any other lens to create omnidirectional sensitivity to the IR sensor or IR sensor arrays.

The present invention may employ the reflective mirror-like surface of certain OLEDs structures and devices (which is sometimes dependent on construction and, for example, choices of materials used, for example, for the ohmic and/or electrode contacts) as a light reflecting surface for providing directional light from another light source such as an LED, and it may also be used as a mirror surface for a number of purposes including but not limited to reflecting light from, for example, other SSL including LEDs. An example implementation of this is use in a vanity minor that reflects normal visible light when the OLED is turned off, but illuminates when it is turned on that, for example, can also wavelength/color change from white or blue at wake up to amber before bedtime. Another example is combination light containing one or more each of OLEDs and LEDs each of which can be independently controlled, dimmed and monitored, etc.

The present invention is not limited to controlling any single device and is capable of connecting to virtually an unlimited number of devices Likewise multiple solid state lamp/lighting devices may be controlled by a single IR unit with one or more IR emitters or through any single or more than one phone/tablet/computer/smart device, etc. In some embodiments of the present invention, fluorescent lamp replacements are provided including but not limited to T8, T12, and/or T5 linear solid state lighting including LED, OLED, QD, etc. combinations of these, etc. In some embodiments of the present invention, the wireless or wired implementation may be used to provide dimmable, color/wavelength-changing, full or partial spectrum selectable and programmable lighting that can also have IR LED emitter incorporated into the solid state lighting replacement for fluorescent tubes such that one or more IR LEDs at different angles, positions, locations for example on linear fluorescent tubes may be used to remotely wired and/or wirelessly control IR remote control heaters, coolers, air conditioners, humidifiers, televisions, DVD, DVR, VHS, Blu-ray players and recorders, cable and/or satellite receivers, CD players and recorders, other audio-visual and entertainment equipment, etc. In other embodiments of the present invention, lighting that is directly plugged into the AC lines may also may use powerline, wireless and/or wired interfaces that may be used to provide dimmable, color/wavelength-changing, full or partial spectrum selectable and programmable lighting that can also have IR LED emitter incorporated into the solid state lighting replacement for fluorescent tubes such that one or more IR LEDs at different angles, positions, locations may be used to remotely wired or wirelessly control IR remote control heaters, coolers, air conditioners, humidifiers, televisions, DVD, DVR, VHS, Blu-ray players and recorders, cable and/or satellite receivers, CD players and recorders, other audio-visual and entertainment equipment, etc.

The present invention allows automatic, manual, programmable including user-programmable or selectable switchover from linear to duty cycle (e.g., pulse width modulation (PWM)) or duty cycle to linear regulation as a function of either current or voltage on the load (e.g., OLED, LED, QD, other solid state lighting, combinations of these, etc.)

Embodiments of the present invention can track user movements and, for example, light and/or heat the way using for example, but not limited to, motion, proximity, RF, RFID, heat, temperature, sound, pressure, displacement, radar, ultrasonic, infrared, velocity, acceleration, thermal, etc.

In some embodiments, panel lighting is provided including, but not limited to, phosphorescent OLED lighting panels. OLEDs which offer a thin, lightweight, energy-efficient and large-area diffuse source of lighting with excellent visual quality. Compared to fluorescent lighting (FL), OLED lighting panels do not contain hazardous materials. There are aesthetic and visual effects to OLED lighting that are not easily possible to replicate with fluorescent lighting or LEDs. As with LEDs, Phosphorescent OLED lighting devices are current controlled devices. To achieve innovative and imaginative lighting products consisting of multiple panels including non-rectangular shapes, the power supplies can be configured to fully support OLED applications. In particular these power supplies are able to, for example, convert AC input to DC output power, have a high power factor (PF) and low total harmonic distortion (THD), support various types of dimming, meet FCC EMI limits, provide over-current (OCP), over-voltage (OVP), over-temperature (OTP) and short circuit protection (SCP). Also, these power supplies are amenable in some embodiments to form fit applications for OLEDs. Both isolated and non-isolated power supplies for OLEDs support both white light, white-changing and color tunable red/green/blue (RGB) modes of operation. The power supply and design avoids localized heating that may lead to localized degradation of the OLEDs, especially the blue OLEDs, resulting in an unattractive localized yellowing of the part OLED panel(s) in the proximity of the power supply. Two example ways of addressing this potential issue are to: (1) design extremely efficient (i.e., ˜97%) AC to DC power conversion power supplies, and (2) innovatively replace the discrete power supply components that operate at higher temperatures over a number of smaller components that are distributed over a larger area such that there is neither a large variation in temperature nor any hot spots that would raise the OLED display temperature, even locally, by more than a few ° C.

In some embodiments, the simple, low-cost drivers are at least 90% efficient for output powers greater than 10 Watts, have PF >0.95 (typically 0.98+), and THD <20%. The smart drivers, in addition to the performance specified for the simple drivers can support, among others, optional wall (Triac), 0 to 10 V, powerline (PLC), wireless, DMX and DALI dimming. In addition to versions that support white light dimming via smart phones, tablets, iPods, iPads, iPhones, Android devices, Kindles, computers, etc., RGB, WRGB, WRGBA, RGBA, etc. color/mood changing LED, OLED, QD and/or other SSL panels are also supported via the same interfaces and mobile/computer devices which can also provide white light. Examples of control and monitoring system using iPhones, iPads and iPods to control and monitor the light color and light (dimming) level are showed below. In other embodiments of the present invention blue and amber LEDs, OLEDs, QDs, other SSL, etc., and/or combinations of these, etc. can be used to provide white color as well as blue color or amber color so as to provide appropriate lighting for various times of the day which could, for example, support healthy lighting options including lighting to support circadian rhythms, seasonal affective disorder (SAD), etc. In an example embodiment, blue and amber OLEDs can be integrated and incorporated into the same lighting panel and each color is independently controllable such that the blue and amber OLEDs—or other lighting sources such as quantum dots (QDs)—can be independently controlled, adjusted, dimmed, turned on or off, etc. for example by having one or more separately addressable electrodes, contacts, etc. For example, the lighting can be set to white (or blue wavelength/color-enriched) in the morning and set to amber at night for people and animals on a more typical circadian rhythm cycle and the lighting can be set to white (or blue wavelength/color-enriched) in the afternoon, or night or other appropriate time(s) and set to amber at later night or late night (with the time being dependent on the individual's particular schedule including but not limited to work schedule, etc.) for people and animals on a non-normal circadian rhythm cycle.

This example RGBW and power management control and monitoring system operates with virtually any smart phone, tablet, laptop, computer, server, etc. to, for example, dynamically separately select and control any number of lights including controlling light level (dimming), power factor, power/energy usage (i.e., kWH), input and output current, voltage, etc. The cost of ownership and the cost of implementation are relatively low for this system yet extremely flexible and powerful including high efficiency low and high power drivers that are adaptable and support many forms of dimming, monitoring and control. graphical user interface (GUI) pages as well as a very large number of user-adjustable and selectable and custom colors can be used with the present invention. A graphical user interface can be provided on one or multiple control devices, such as, but not limited to, smart phone, tablet, laptop, computer, server, remote control, etc. Such a graphical user interface can support the selection and control of devices on an individual basis or in groups, for example grouped by area, room, type, user-defined groups of devices, etc. The graphical user interface enables the user to turn devices on and off, to set dimming levels, colors, etc. For example, a color wheel is provided in some embodiments to select lighting colors. In some embodiments, predefined colors can be selected. In some embodiments, RGBW or CMYK, etc., values can be entered using numerical entry, icon selection, movement of graphical input icons such as sliders, knobs, etc., touch gestures such as swiping to vary settings, etc. The graphical user interface also enables the user to save settings individually or by groups, to name settings, to share settings with other users, to store and retrieve settings, to schedule settings changes based on time, day, sensor inputs, etc. In some embodiments, the graphical user interface also provides user feedback of device status such as, but not limited to, on/off state, power factor, power efficiency, color, upcoming scheduled events, etc. In some embodiments of the present invention, this particular suite of GUIs, in addition to controlling and monitoring the light, etc. also controls and monitors temperature including room and local temperature, humidity, entertainment status including whether the television is turned on, the channel, show, volume, etc., selected, the temperature in various locations and spaces in the house, residence, apartment, building, condo, office, school, etc. the heater, cooler, air conditioner on/off/level/ power consumption, etc., combinations of these, etc.

Unlike simple infrared controlled RGB lightstrips, ropes and the likes with limited color choices and dimming levels, the low-cost RGB lighting allows for high resolution 8-bit to 12-bit (256 to 1024, 2048, 4096, etc.) or higher resolution color levels per RGB channel and with innovative ways to interactively and dynamically user-select the resolution and dimming level. The present invention can also be used for WRGB. WRGBA, RGBA, etc. lightstrips, strings, ropes, etc.

Highly innovative and novel flexible and adaptable OLED or QD large or larger area replacements for fluorescent lamp luminaire retrofitting including both ballast-less (i.e., OLED power supply directly connected to AC lines) replacement and ‘drop-in’ socket replacement (i.e., T-8 or T-12 OLED power supply directly connected to either a magnetic or electronic ballast in place of the fluorescent lamp tube)—are provided, such as the example T8 or T12 fluorescent lamp conversion using an OLED and/or LED retrofit ‘kit’ which, for example, can be made up of either WOLED or RGB OLED panels and in some implementation LED panels that are ‘stitched’ together to form a flexible area panel. In this example embodiment, four 4 foot long T8 FLs are replaced by OLED area lighting which may be a single panel or a group of stitched panels with an OLED power supply that is designed to plug either directly into the ballast(s) for the T8 FLs or into the AC mains (or both) so as to make it easier to retrofit and install (when the ballast eventually fails, the ballast can be removed and the OLED power supply can be plugged directly into the AC lines. Other embodiments can include other SSL including but not limited to LED, OLED, QD, etc., combinations, etc. of these. The OLED/LED/QD/SSL retrofit including the power supply can be hung/suspended (like a false ceiling) from the FL luminaire or, for example, the OLED/LED/QD/SSL power supply can be inserted in place of the ballast and the OLED/LED/QD/SSL stitched panel can be attached/suspended from the OLED/LED/QD/SSL power supply and drivers. The present invention can also be used with Edison sockets such as A-lamps, PAR 30, PAR 38, MR 16, etc. as well as high intensity discharge (HID) including but not limited to sodium discharge lamps, mercury vapor lamps, metal halide (MH) lamps , ceramic MH lamps , sodium vapor lamps, xenon short-arc lamps, ultra high pressure lamps (UHPs), other types of gas and metal-halide and/or metal salts, etc. Edge emitting solid state lighting (SSL) including edge emitting LEDs and/or Edge-Lit LEDs can be used with the present invention.

Another embodiment provides for highly flexible and adaptable SSL/LED/OLED/QD replacement area lighting that in some embodiments is extremely easy to install and suspend with gravity leveling the SSL/LED/OLED/QD panels and the associated power supply and drivers supported by, for example, the fluorescent luminaire/fixture by a number of secure methods. In addition, the OLED panels do not need diffusers typically used with fluorescent luminaires. Also, innovative color changeable RGB OLED and/or QD and/or LED fluorescent replacement retrofitting lighting (with associated OLED RGB power supplies) can be readily implemented with this approach that can be dual use (i.e., white or user-selectable color) without compromising performance, efficiency, efficacy, etc. In some embodiments, an OLED or, for example, an OLED/LED A-lamp can swivel about the axis of the socket. The internal power supply is contained within the socket. The internal drivers are dimmable, high efficiency and high PF. In some embodiments, a white LED and an amber OLED are used to provide white light ‘daylight’ and amber light ‘nightlight’ to support, for example, circadian rhythms and other health effects at work places, homes, hospitals, etc.

In some embodiments, a vertical version of the OLED or LED/OLED A-lamp is provided with the internal OLED and LED drivers inside the A26 lamp socket and a round plastic cover cylinder attached between the socket on the OLED panel. Another version of the OLED A-lamp includes two back-to-back OLED panels powered by internal driver(s). The internal drivers are dimmable, high efficiency and high PF. Embodiments of the present invention may also use motors, actuators, etc. to tilt, move, angle, etc. the OLED (or LED or both) lighting. In other embodiments of the present invention, the OLEDs may be replaced or augmented with either white LEDs (or any other color) or RGB LEDs to perform the T8, T12, T5, U shaped or other fluorescent lamp replacement, etc. Other embodiments of the present invention may employ wireless power transfer such as inductive coupling or resonant coupling to remotely power the OLEDs or LEDs.

In some embodiments, a motorized, telescoping mount or support is provided for a lighting panel, enabling the light to extend to a desired position and direct the light in a desired direction, and then to retract into a compact configuration when not in use or when commanded. In some embodiments, the retracted configuration provides protection for the lighting panel, either within a housing or using segments of the telescoping mount for protection. Such embodiments may also be portable and may also have personalized settings such that the lamp returns to the same height, angle, etc. (i.e. location) when turned on or commanded to do so. Any number of such personalized settings from 1 to N where N can be a very large number can be incorporated and be part of the present invention.

The present invention may be integrated with other forms of automation, control, monitoring and management of energy and power, etc.

The present invention may have speakers placed inside standard light bulb formats such as T8, T10, PAR30, etc. These light/speaker combos, or simply speaker assemblies may be wired or wireless, connecting to various electronic systems capable of providing audio signals. Methods of communication include Bluetooth, radio, LAN, RS-232, SPI, I2C, other forms of serial interfaces, DMX, DALI, etc. and other forms and protocols of communication. Embodiments and implementations of the present invention can include the inclusion of speakers and/or microphones into the light devices which provide experiences where both light and sound interact together. For example, the light amplitude may be correlated to audio amplitude, so that the intensity increases when the music is louder. Other situations for such a device include setting up a home theater with 5.1, 7.1, etc. surround sound in which simply replacing the lights with the speaker-enabled lighting device can be used instead. A full home surround system can be implemented using this present invention.

The present invention may be in the form of non-standard lighting solution such as but not limited to wall surfaces, minors, floor tiles, automotive head lamps, curved surfaces such as rotating cylinders and furniture components.

The present invention can coordinate with other storage/charging systems including wall mount, desk furniture (which may also have, for example, solar cells attached to, for example, but not limited to sofas, chairs, seats, tables of any type, desks, shelves, ottomans, love seats, beds, head boards, dressers, pianos, foot boards, end tables, bookshelves, floors, walls, ceilings, etc. so as to be able to optimize/maximize the energy storage and usage.

The present invention can support all standards and conventions for 0 to 10 V dimming or other dimming techniques including but not limited to DALI, DMX, RS232, RS485, other serial interfaces and protocols, etc. In addition the present invention can support, for example, but not limited to, overcurrent, undervoltage, overvoltage, short circuit, under current, and over-temperature protection.

The electronics for converters, inverters, direct current to alternating current (i.e., DC to AC), chargers, distribution, etc. in general may use and be configured in continuous conduction mode (CCM), critical conduction mode (CRM), discontinuous conduction mode (DCM), resonant conduction modes, etc., with any type of circuit topology including but not limited to buck, boost, buck-boost, boost-buck, cuk, SEPIC, flyback, forward-converters, etc. The present invention works with both isolated and non-isolated designs including, but not limited to, buck, boost-buck, buck-boost, boost, flyback and forward-converters. The present invention itself may also be non-isolated or isolated, for example using a tagalong inductor or transformer winding or other isolating techniques, including, but not limited to, transformers including signal, gate, isolation, etc. transformers, optoisolators, optocouplers, etc.

The present invention may include other implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, digital signal processors (DSPs), application specific integrated circuits (ASICs), etc. can also be used either alone or in combinations including analog and digital combinations for the present invention. Parts of the present invention can be incorporated into an integrated circuit, be an integrated circuit, etc.

The present invention may be used with a linear regulator, a switching regulator, a linear power supply, a switching power supply, multiple linear and switching regulator and power supplies, hybrid linear and switching regulators, hybrids of these, combinations of these, etc.

The present invention can also incorporate at an appropriate location or locations one or more thermistors (i.e., either of a negative temperature coefficient [NTC] or a positive temperature coefficient [PTC]) to provide temperature-based load current limiting.

The present invention can also be used for purposes and applications other than discussed above.

The present invention can also include circuit breakers including solid state circuit breakers and other devices, circuits, systems, etc. that limit or trip in the event of an overload condition/situation. The present invention can also include, for example analog or digital controls including but not limited to wired (i.e., 0 to 10 V, RS 232, RS485, IEEE standards, SPI, I2C, other serial and parallel standards and interfaces, UARTS in general, etc.), wireless, powerline, powerline communications (PLC),etc. and can be implemented in any part of the circuit for the present invention.

Embodiments of the present invention can use comparators, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices, field programmable gate arrays, etc.

The present invention includes implementations that contain various control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc.

The example embodiments disclosed herein illustrate certain features of the present invention and not limiting in any way, form or function of present invention. The present invention is, likewise, not limited in materials choices including semiconductor materials such as, but not limited to, silicon (Si), silicon carbide (SiC), silicon on insulator (SOI), other silicon combination and alloys such as silicon germanium (SiGe), etc., diamond, graphene, gallium nitride (GaN) and GaN-based materials, gallium arsenide (GaAs) and GaAs-based materials, etc. The present invention can include any type of switching elements including, but not limited to, field effect transistors (FETs) of any type such as metal oxide semiconductor field effect transistors (MOSFETs) including either p-channel or n-channel MOSFETs of any type, junction field effect transistors (JFETs) of any type, metal emitter semiconductor field effect transistors, etc. again, either p-channel or n-channel or both, bipolar junction transistors (BJTs) again, either NPN or PNP or both, heterojunction bipolar transistors (HBTs) of any type, high electron mobility transistors (HEMTs) of any type, unijunction transistors of any type, modulation doped field effect transistors (MODFETs) of any type, etc., again, in general, n-channel or p-channel or both, vacuum tubes including diodes, triodes, tetrodes, pentodes, etc. and any other type of switch, light/optical/temperature/humidity/pressure/force/position sensing device, circuit, system, etc solar cells including all types and makes or photovoltaic, hybrid, etc. solar cells.

Embodiments of the present invention are related to systems, apparatuses, and methods for energy collection, storage and/or usage from solar panels or collectors embedded in or mounted on curtains, drapes, shutters, shades, blinds or other structures adapted to control, for example, but not limited to sunlight, other sources of light or other parts of the electromagnetic spectrum, and/or artificial light/solar/electromagnetic energy through a window or incident on another area such as a patio or porch. Solar panels are provided on window coverings such as, but not limited to, curtains, drapes, shutters, shades, blinds or other shade structures such as awnings or automatic solar shades including sun blocks used in recreational vehicles such as motor homes, camper trailers and temporary housing structures. The solar panels can be any device or material known or that may be developed in the future for converting light to electricity in any manner. The solar panels can be mounted or attached in any suitable manner, whether permanent or removable. For example, solar panels are attached to fabric curtains in some embodiments by an adhesive, or by sliding them into partial pockets that hold the solar panels at the edges and/or corners but that expose the active light collecting surfaces. In some embodiments, attachment points are provided on the solar panels, such as holes at the edges or corners through which fasteners are attached between the solar panels and the curtains or other shade material.

Various embodiments of the invention include one or more of the following features:

The present invention can be used to harness solar energy.

The present invention can provide protection from IR and UV light while providing energy/power.

Embodiments and implementations of the present invention can be manual, motorized, automatic, etc.

Embodiments and implementations of the present invention can come in numerous sizes and shapes

Embodiments and implementations of the present invention can be rotated to allow partial or full light through window or to track and maximize solar energy transfer.

Embodiments and implementations of the present invention can use flexible solar cells

Embodiments and implementations of the present invention can provide wired and/or wireless control and monitoring including, but not limited to, powerline control, RS232, USB, SPI, SPC, I2C, etc., WiFi, Bluetooth, ZigBee, IEEE 801, ISM, etc.

The remote control can, for example, open/close/raise/lower/rotate/tilt/etc. the respective shade, drapes, shutters, curtains, blinds, etc.

Embodiments and implementations of the present invention can be connected to energy storage devices including batteries and fuel cells including but not limited to in cosmetically attractive ways.

Embodiments and implementations of the present invention can respond to sound, motion, other light sources, etc.

Embodiments and implementations of the present invention can be activated by dusk and dawn scenarios and situations.

Embodiments and implementations of the present invention can monitor and report energy usage.

Embodiments and implementations of the present invention can be used to charge and provide power to portable and other devices including cell phones, tablets, smart phones, flash lights, cameras, tablets, iPads, iPods, laptops, televisions, telephones, radios, Internet, Web, DVD, DVR and other media players, desk top computers, stereo systems, lights, lamps, etc. As an example, a standard land line telephone or non-smart phone can also be used to provide the same or similar control and even monitoring of the present invention by sending digital signals such as tones including but not limited to two tone modulation signals including, if desired, password protected information or encrypted information, to set, turn on, turn off, dim, increase, monitor, control, etc. implementations of the present invention. Other embodiments may use light sensors including color spectrum light sensors to sense, detect, measure, etc. and control the color and/or color temperature of the lighting including solid state lighting (SSL) such as, but not limited to, light emitting diode (LED), organic light emitting diode (OLED), quantum dot (QD), etc. lighting. A spectrum sensor/detector, etc. could consist of color filtered charge coupled devices (CCDs), notch filters, QDs, including QD single or multiple color/wavelength QDs including red green blue wavelength (RGB) QDs, full spectrum QDs, red green blue amber (RGBA) QDs, four color/wavelength QDs, five color/wavelength QDs, more than five (5) color/wavelength QDs, filters, etc.

Embodiments and implementations of the present invention can be used as an emergency source of energy.

Embodiments and implementations of the present invention can be set to automatically open and close as person(s) enter and exit.

Embodiments and implementations of the present invention can charge batteries for portable devices and other items needing batteries.

Embodiments and implementations of the present invention can be equipped with emergency/non-emergency lighting capabilities such as LEDs or any other light emitting devices including other types of solid state lighting for use during power outages or normal lighting periods.

Embodiments and implementations of the present invention can be activated to open at dawn and/or at a prescribed time as an wake-up system or part of an alarm system including a protection or alert system. The present invention can be used to schedule and sequence turn-on, turn-off, dimming, increasing, decreasing, channel changing, recording, monitoring, control, etc. of any number of devices, appliances, heaters, coolers, fans, air conditioners, furnaces, humidifiers, dehumidifiers, etc., in any combination and combinations of sequences and scheduling including time scheduling with set or variable or specific set or variable timing and duration that can be, for example, user selected, sequenced and/or scheduled or automatically selected, sequenced and/or scheduled.

Embodiments and implementations of the present invention can be used to provide privacy both during the day and at night

Embodiments and implementations of the present invention can also charge at night using lighting sources such as street lamps, outdoor lighting and other sources of human-made or natural solar/light/optical energy, etc.

Switches can be set to route and switch power from the solar shades, panels, curtains, drapes, shutters, blinds, etc. to batteries and other storage devices, cells, etc., to DC to DC converters, DC to AC inverters, AC to AC inverters, etc. other power sources, other power storage, converters, consumers, users, including power consumers such as heaters, coolers, air conditioners, fans, etc., and/or to the power grid, etc. Such power switching and direction and bidirectional power transfer, movement, distribution, redistribution, etc. can be accomplished wirelessly, wired, by powerline, by combinations of these, etc., by manual and/or automatic operation, including automatic decision making, algorithms, other forms of remote control, etc.

Embodiments and implementations of the present invention can include smart and intelligent power inverters and converters including wired, wireless, powerline remote controlled and monitored with analytics.

Embodiments and implementations of the present invention can be used for alert, home and/or business protection by including motion sensors or other intrusion detection which transmits status through the system.

Embodiments and implementations of the present invention can be used for fire detection, smoke detection, carbon monoxide detection, gas detection including but not limited to natural gas detection by including the appropriate detectors or sensors which transmit status through the system.

Embodiments and implementations of the present invention can monitor for water, moisture leaks and optionally being able to turn off water by including moisture sensors which transmit status through the system and optionally including automated valves which can be opened and closed by remote commands transmitted through the system.

Embodiments and implementations of the present invention can provide remote operation and monitoring for physically or mentally impaired.

Implementation of the present invention can perform powering on or off or optionally dimming, depending on the type of load, including remote wired, wireless, and/or powerline, appliances, heaters, coolers, fans, HVAC, air conditioners, furnaces, central air, humidifiers, dehumidifiers, TVs, entertainment centers, Cable boxes, satellite boxes, gaming boxes, DVD, Blu-Ray. DVRs, VCRs, CDs, audio and/or video tapes, stereo players, record players, amplifiers, radios, including frequency modulated (FM) and amplitude modulated (AM) mono and stereo radios, as well as weather, shortwave and/or other radio frequencies, ranges, bands. etc., switching networks, switches including IR controlled audio visual (AV) switches, and monitoring power usage, time usage, user usage, efficiency, etc.

Elements of the system can be installed in place of light switches, lamp controls, fan controls, AC line outlet boxes, junction boxes, etc.

In some embodiments, the solar cell shades/blinds/curtains etc. provide control functions such as those disclosed in U.S. patent application Ser. No. 13/795,149, filed Mar. 12, 2013 for a “Solar Powered Portable Control Panel”, which is incorporated herein by reference for all purposes. For example, the solar cell shades/blinds/curtains etc. can incorporate control and sensor functions or interface with controllers and/or sensors and/or loads such as those described in the above-referenced patent application, including lighting applications and sensor-based control.

The solar panels can be embedded in or attached to the curtain/blinds/shades in any manner, and can be any type of solar energy collectors and/or converters, including high efficiency panels as well as flexible panels or light-sensitive threads, strings, fabrics or other materials. The solar panels can be applied to flexible structures such as fabric curtains, shades or blinds, or to rigid structures such as blinds, shutters and/or shades. The solar panels can be used in window coverings or other types of coverings for other areas such as awnings or retractable shades to control sunlight to patios or porches. The settings, position, power/energy generation/conversion, open or close or degree of open or close, temperature, light level, etc. power level, battery and other energy storage states and levels, analytics, etc. can be remotely set, conditioned, controlled, monitored, etc.

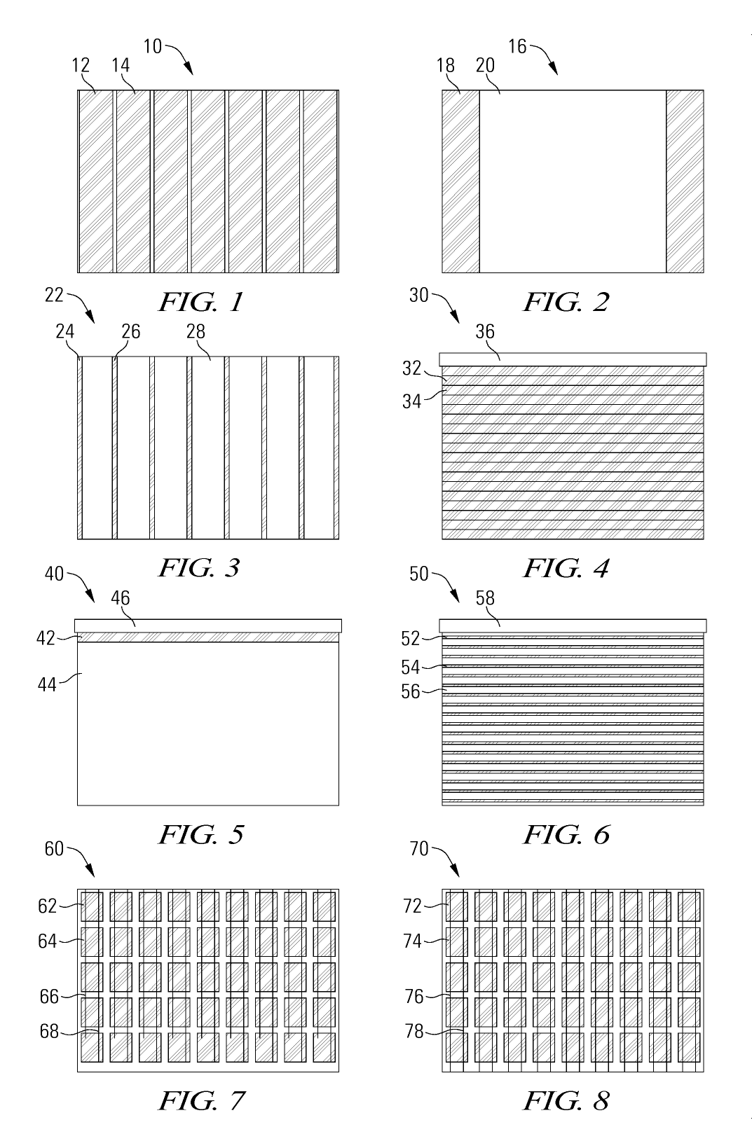

Turning to FIG. 1, a window 10 is depicted with closed window coverings or vertical blinds (e.g., 12, 14) with solar collectors embedded therein or mounted thereon in accordance with some embodiments of the invention. In this and every embodiment, the solar collectors can be any suitable device for collecting solar power, including but not limited to solar panels, solar cells, etc. Furthermore, in this and every embodiment, the window coverings (e.g., 12, 14) can be any type of window covering in any fabric or plastic or wood or other material, such as, but not limited to, vertical blinds, drapes, curtains, etc.

A window 16 is depicted in FIG. 2 with open solar collecting blinds, shutters, drapes, or curtains, etc. in accordance with some embodiments of the invention, in which the blinds shutters, drapes, curtains etc. 18 with solar collection devices mounted therein or attached thereto in any manner are pulled back, exposing much or all of the window pane 20.

Turning to FIG. 3, a window 22 is depicted showing another example window covering system including horizontal blinds (e.g., 24, 26) with solar collectors embedded therein or mounted thereon, which are rotated around a vertical axis to an open position to expose much or all of the window pane 28.

Turning to FIG. 4, a window 30 is depicted with another type of window covering system in a closed position, including horizontal solar shutters or blinds (e.g., 32, 34) with solar collectors embedded therein or mounted thereon. In some embodiments, a decorative valance 36 or a box into which the solar shutters or blinds (e.g., 32, 34) can be pulled is provided.

Turning to FIG. 5, a window 40 is depicted with horizontal solar shutters or blinds (e.g., 42) pulled up into an open position behind a valance 46, exposing window pane 44.

In FIG. 6, a window 50 is depicted with horizontal solar shutters or blinds (e.g., 52, 54) each rotated around a horizontal axis into an open position, exposing much or all of window pane 56. The wiring can be between each blind, shutter, etc., and can for example, be incorporated as part of the manual or automatic mechanical support for each blind, shutter, shade, etc.

In FIG. 7, a window 60 is depicted with example strings of solar cells or panels (e.g., 62, 64) forming a window covering in accordance with some embodiments of the invention. The wiring (e.g., 66, 68) can be between each cell/panel that effectively forms the blind, shutter, drape, etc., and can, for example, be incorporated as part of the manual or automatic mechanical support for each blind, shutter, shade, etc.

In FIG. 8, a window 60 is depicted with example strings of solar cells or panels (e.g., 72, 74) forming a window covering in accordance with some embodiments of the invention. The wiring (e.g., 76, 78) can be between each cell/panel that effectively forms the blind, shutter, drape, etc., and can, for example, be incorporated as part of the manual or automatic mechanical support for each blind. The wiring can be from the top, one or both sides, the bottom and/or combinations of these.

Embodiments of the present invention can have/use solid state lighting including, but not limited to LEDs, OLEDs, Quantum Dots (QDs), etc. as well as other types of light sources/light producing/generating lighting on the other ‘side’ (i.e., the interior/room facing) instead of the outside facing which has the solar cells/panels. In other embodiments, there may be panels on both sides. The solar cells/panels may be made up/consist of different types of, materials, homojunction, heterojunction, single crystal, poly-crystalline, amorphous, flexible, etc. solar and/or photovoltaic systems, materials that collectively cover all or part of, for example, the deep infrared through the infrared, through the infrared, through the visible, to the ultraviolet and even the deeper ultraviolet. In other embodiments, systems that absorb and use other parts of the electromagnetic spectrum including but not limited to radio frequencies (RF), microwave, millimeter-wave, sub-millimeter wave, terahertz, etc., long wavelengths, the AM and FM, short-wave, etc. to harvest additional power and potentially also block wireless signals from penetrating the window covering, shade, curtain, drape, blind, shutter, etc. In other embodiments, the solar and/or other energy absorber may be integrated and/or incorporated onto the same substrate either heterogeneously or homogeneously; for example, GaN-based LEDs and solar cells, GaAs-based LEDs and solar cells, OLED lighting and OLED solar cells, QD lighting and QD solar cells, other solid state lighting and solid state solar/photovoltaic cells, other types of materials and structures for lighting and electromagnetic absorption, transmission, generation, conversion, etc. The solar/photovoltaic/etc. cells/panels can be wired/connected in parallel, series, combinations of these, etc. The cells/panels can be square, rectangular, circular, elliptical, odd shaped, irregular shaped, essentially two dimensional, three dimensional, spherical, hemispherical, cylindrical, parallel piped, etc.

The automation system is adapted in some embodiments with motors or other actuators to open and close window coverings such as, but not limited to, curtains, blinds, drapes, louvers, shutters, etc. Such automated opening or closing can be scheduled and/or triggered based on sensors, etc. For example, window coverings can be programmed to automatically close at a particular time each evening, or when a light sensor indicates low light conditions at dusk or sundown. Window coverings can be programmed to automatically and gradually open in the morning or at a desired waking time in a bedroom to gradually awaken sleepers or at a particular light level or in combination with time, light, other stimuli and information or subsets of these or other combinations or by individual selections, etc. Window coverings can incorporate sound reduction, sound absorption, or other sound proofing materials to reduce outside noise. Window coverings can be provided with interior lighting devices or panels, for example to provide room lighting while continuing to partially or fully cover and block a window. Solar collectors on the outside of a window covering can collect power from sunlight, streetlights, or other light sources outside while powering lighting on the inside of the window covering, allowing more preferable lighting with desired color, wavelengths, illumination levels, etc. to be powered and provided on the inside of the window covering, thereby providing privacy and energy efficient lighting and dampening noise from outside.

In some embodiments, a smart phone or other mobile control device is used to control a space heater or other heating and/or cooling system, referred to herein as a temperature controlling device, or other elements of a home automation and/or home entertainment and/or home control system. In some of these embodiments, the smart phone, tablet, including but not limited to iPod, iPad, iPhone, remote control or other mobile control device includes a temperature sensor and is operable to provide temperature measurements as part of a thermostat system. In some cases, the temperature sensor in the smart phone or other mobile control device controls the space heater, fan, portable air conditioner, window air conditioner, evaporative cooler, combinations of these and other types heating, cooling, flow, HVAC, registers, vents and/or ducts, etc. based on the temperature at the location of the smart phone, rather than at the location of the space heater. In some cases, the temperature at multiple locations is measured and used to control the space heater or other heating and/or cooling system, for example including multiple smart phones in the system to control the temperature at multiple locations in a residence. The temperature at the location of the heater can also be included in the control algorithm, for example limiting the temperature at the location of a space heater so that it does not exceed a maximum temperature.

Such a “mobile thermostat” can be used to control a heating and/or cooling system in a residence to substantially heat or cool only the locations used by occupants based on temperature measurements by for example, remote controls, smart phones, tablets, laptops, personal digital assistants, other portable digital assistants, voice commands, voice recognition, motion detection, proximity detection, pressure detection, RFID, Bluetooth, other signal strength detection, GPS, smart phones etc. on or near the occupants. In some embodiments, the heating and/or cooling system can be configured to maintain a basic default level of heating or cooling elsewhere. Such “mobile thermostat(s)” can also be employed in any location within a dwelling, residence, home, office, building, warehouse, etc. and be connected with the HVAC system via wired, wireless, power line control, etc. In some embodiments of the present invention, the mobile thermostats can be powered wirelessly by solar, mechanical, vibration, radio frequency (RF), infrared, other forms of energy harvesting, etc. and can also use batteries that are charged by these various energy harvesting including, but not limited to solar cells, and combinations, etc. of solar cells and other energy harvesting. One or more of these “mobile thermostats” may be employed at any given time including a mixture of “mobile thermostats” consisting of cellular phone(s), smart detectors, tablets, laptops, etc., portable and/or fixed sensors, etc. and any combination of these that can be static or dynamically changing, etc. These “mobile thermostats” can coordinate with the master (or one or more master thermostats that control one or more furnaces/air conditioners, air blowers, fans, evaporation/evaporative (swamp) coolers, electrical heaters, baseboard heaters, radiative heaters, under floor covering heaters, ceramic heaters, quartz lamp heaters, two- and tri-fuel (including, for example, but not limited to one or more of propane, butane, natural gas, hydrogen, fuel cells, electricity, solar heaters, solar coolers, boilers, portable heater of any type, air conditioner(s) of any type including central air, window air conditioners, roof top units, radiant heaters, radiant floor heaters of all types and kinds, natural gas heaters, propane heaters, other types of fuel heaters, water heaters, hot water heaters, electrical heaters of any type, oil heaters, wood heaters, pellet heaters, hot air heaters, ceiling fans (including the direction of rotation and speed of rotation for ceiling and any other type of fan) etc. which, in some embodiments, can be coordinated with each other including coordinated in a user defined way, approach, model, algorithm, etc. Embodiments of the present invention can also take into account weather related and temperature and temporal timing events and effects that affect the heating and/or cooling and proximity including location and attributes such as location, number of windows, multiple sense/sensor points etc. Powered fans, HVAC registers, residential registers, vents and ventilation in general and heater/cooler vent shutters and ducts can be used to direct, restrict, limit, enhance, decrease, increase, etc. the temperature of various locations within a dwelling, either autonomously, or collectively coordinated, manually, automatically, user command selectable, etc. Such fans, registers and vents and in general ventilation can be powered with solar, batteries, other energy harvesting, wireless energy transfer, inductive coupled power transfer, inductive coupled wireless power transfer, AC line, etc. Example embodiments can include, but are not limited to, wireless/inductively coupled power transfer from the solar cell shades to the distribution/power charging of batteries, fuel cells, mechanical energy storage systems such as momentum flywheels, and other types of energy storage that can be used to charge cell phones, smart phones, tablets, uninterruptable power sources/systems (UPSs), computers, servers, routers, lights, smart phones, shavers, radios, music and movie players including, but not limited to, MP3, Ipod, DVD, DVR, VCR, Blu-ray, etc., flashlights, work lights, desk lamps and lights, task lamps and lights, emergency lights, etc. and in general any type of device that requires energy to operate including but not limited to the controller and monitor for the embodiments of the present invention system including, but not limited to, motors, actuators, sensors, detectors, data logging, analytics, etc. for the solar shades and HVAC fans, vents, registers, etc.

Solar cells of any type and/or any material and/or any structure and/or any efficiency can be used with the present invention. Such solar cells include, but are not limited to, silicon (Si)-based, gallium arsenide (GaAs)-based, gallium nitride (GaN)-based, other Group IV, Group III-V, Group II-VI, OLED cells, quantum dot cells, etc. semiconductor and light sensitive materials, heterojunctions, heterostructures, combinations of these, etc. Multiple types of solar cells that include and cover multiple bandgaps/ wavelength ranges/etc. may be used to together or separately to cover various regions of the spectrum including, but not limited to the infrared (IR), visible, ultraviolet (UV), etc. regions. The solar cells may be mixed and matched and may be part of a solar tracking system that changes the position, angle, or other attribute of the solar cells to obtain the desired (i.e., maximum or optimum) response and may include maximum power point tracking (MPPT) and other such methods to maximize and optimize the energy/power transfer, etc.

The present invention can use any type of sensors, detectors in any number and any combinations from simple to advanced, sophisticated and complicated including but not limited to temperature, light, solar, position, inclination, speed, location, acceleration, etc., again in any combination or use. Such sensors include but are not limited to thermocouples, thermistors, platinum-based temperature sensors, resistance temperature detectors (RTDs), semiconductor, integrated circuits, micro-electro-mechanical systems (MEMS), gyroscopes, global positioning systems (GPS), triangulation, sound, electrical (including to measure/detect/monitor electrical parameters such as input and output voltage, current, power, power factor, harmonics, distortion, total harmonic distortion (THD), etc. for example both collectively and individually) mechanical, vibrational, wind direction, strain gauges, moisture, humidity, radio frequency (RF) detectors and sensors including but not limited to radio frequency identification (RFID), infrared detectors, spectrum analyzers and detectors/sensors, time detectors including ones that can detect and use atomic clock signals sent by radio transmitters, etc. Any, a subset or all of these detectors and sensors may be used in any combinations with the present invention. Uses include but are not limited to solar/light detection and response, data logging, analytics, predictions, simulations and modeling, movement and actuation of any type or form including motor, piezoelectric, air/pneumatic, tilting, angling, rotating, etc. including, but not limited to, as part of the energy control, monitoring, management, logging of various embodiments of the present invention.

In an embodiment of the control unit, environmental conditions such as temperature, humidity, barometric pressure, dew point and luminance may be recorded to be used for interpreting the climate both inside and outside of structures, for the use of determining if a disaster has occurred such as flooding, fire, freezing temperatures, etc, for determining the insulating efficiency of windows, walls and other structures designed to insulate or otherwise block the outside climate from the inside. The control unit may be linked to devices such as motorized windows, automatic doors, thermostats, HVAC systems, motorized shades and other devices to adjust the climate inside automatically and intelligently. The sensors may also be integrated into a WIFI or LAN network and displayed in a web terminal or other device to show the status of a building and to identify areas of potential problems. The information may be uploaded to remote servers and combined with other data from other buildings/structures/elements to build a picture of complete building/site efficiency.

The control unit may use algorithms intended to allow the tracking and location determination of individuals inside a certain area. Methods such as RSSI and other techniques may be used to determine motion and occupancy. This information may be used for remote monitoring of the physically impaired to ensure that there is movement in the home/area/building. The system may also be used in situation where the mentally/physically impaired need to be located and their movements tracked in a non-contact non-observable manner. Actions that may be monitored include standing/sitting/dwelling in a certain location and the duration of the action, location relative to the sensors and triangulated in 3D space, and other actions. Movement that is sensed may also be used to detect intruders into restricted areas and for home automation such as turning on a home theater system when one enters a room, etc.

Energy derived from the wind may also be used, incorporated, integrated into the present invention including, for example, providing additional sources of energy and power including both during the day and during the night. Wind energy can be used to, for example, drive generators to produce electrical and/or mechanical energy which can be consumed, stored, etc. as part of the present invention.

Fast warm up heaters, heat guns, hot air guns and blowers, personal and larger heaters (including but not limited to quartz heaters, vortex, convection, conduction, parabolic, infrared heaters or all types)and fans and air conditioners and other methods of heating and cooling can be used to provide single or multiple users personal comfort settings and levels as part of the present invention. The present invention can also provide global, group and/or individual humidity levels based on persons and/or locations. Hot air dryers for hands can be adapted and used with the present invention to provide quick and nearly instant heat where needed and/or desired. The settings, position, temperature profiles and/or locations, open or close or degree of open or close, temperature, light level, etc. power level, other temperature and/or energy states and levels, analytics, etc. can be remotely set, conditioned, controlled, monitored, etc.

Two or more solar shades/drapes/curtains/blinds/shutters/etc. of the same, similar or different types can be coordinated and communicate with each other either directly or via the control, monitor, management system of the present invention to provide the desired/required energy profile. This includes solar shades that may have different light/solar exposures for whatever reason (including, but not limited to facing different directions or angles including, as examples, facing east and south or east, south and north, east, southwest and northeast, for whatever reasons (i.e., method of construction, architecture, part of a set of windows including bay style windows that are at different angles from each other, etc., windows in an room/area/etc. that are on/located on different walls including, but not limited to walls that are at right angles to each other, sunlights, roof windows, dormers, French doors and other types of glass patio doors, etc. Embodiments of the present invention that involve one or more solar shades/drapes/blinds/shutters/curtains can coordinate in any desired fashion including partially or completely opening or closing one or more of the solar shades/drapes/blinds/shutters/curtains while one or more other solar shades/drapes/ blinds/shutters/curtains are partially or completely closed or opened, respectively, etc. and can be statically or dynamically set, controlled, monitored, changed, etc, depending on any selected conditions, scenarios, situations, etc. including movement of the Sun, change in illumination due to, for example, time of day, weather (including clouds, rain, fog, etc.), change in direction, weather, wind, etc.

Status updates, faults, control, monitor, interrupt, changes, system updates, etc. can all be set, monitored adapted, modified, enhanced, controlled, etc. with the present invention including locally and remotely. Voice, e-mail, gestures, movement, position, web, smart phone and tablet (etc.) applications (apps), other remote controls and monitoring etc. can be used with the present invention to tailor, optimize, maximize, manage, modify, share, allocate, distribute, balance, transfer (including transferring and interacting with power grids), etc. energy/power for the present invention. The present invention can also be used with social media and other such internet software such as Facebook, Google, Instagram, Twitter, etc. and in conjunction with security and fire protection systems, etc., entertainment, cable and satellite systems and providers and to provide assistance and monitoring to and for elderly, senior, disabled person(s), etc. including remote assistance that can be controlled, monitored, managed, etc. by others remotely.

The control system can be used to control entertainment sources including providing custom apps, web pages and/or social media interfaces that combine various media (i.e., antenna TV, cable satellite, stereo, DVD, DVR, VCR, Blu-ray, CD, HVAC including but not limited to heating, cooling, humidity, temperature, air flow, air filtration, temperature distribution and profile, etc. onto user-adjustable and selectable apps or web pages that allow the user to immediately set up the entertainment, comfort, work, etc. environment or select from a suite/set of such environments which can be different for any number of users. The present invention can also measure, determine, calculate, expand upon, track, provide analytics, etc. of power consumption, energy-efficiency, power factor, energy costs, etc. by for example, using an energy/power monitoring device or devices to measure either or both the AC and/or DC input power, etc.

A microphone or microphones are used in some embodiments to receive and recognize voice commands to control temperature, entertainment, heating, cooling, lighting, including color temperature and color, etc., opening and closing vents, setting alarms, monitoring, etc.

Embodiments of the present invention can use RFID, other RF signals including but not limited to cell phones, smart phones, tablets, wireless devices in general to track, monitor, log, etc. one or more persons, animals, or objects location, duration, movements, etc. Such tracking, monitoring, logging information can be used in many ways and for many purposes and applications with the present invention including, but not limited to, deciding how to allocate resources, how to set the solar shades/curtains/drapes/blinds/shutters/etc. (including position, angle, open, closed, partially closed or open, etc.), how to distribute light/energy/power among the various components of the present invention, to send alerts if persons, animals, objects spend too long in certain locations or enter locations that they should not be in, etc. One example is if a person is detected as being in a location such as a bathroom for too long of a period, an alert could be sent out and broadcast to select devices and persons including a situation where an elderly person is in the bathroom for a long period of time.

The present invention can also interact with, incorporate and work with medical monitoring, control, sensing and detection devices, equipment and instrumentation and take appropriate actions and broadcast alerts (including to all or a subset of family members, friends, co-workers, emergency services, medical personnel, others, etc.) when a medical emergency is detected such as the onset of a heart attack, a person suddenly falling down, irregular and abnormal monitoring signals, etc. This can be accomplished using either or both wearable or non-wearable devices including wrist, head, ankle, other parts of the body, head, appendages, watches, etc.

The present invention can also work with conventional, non-programmable, or non-remotely programmable fixed location thermostats. As an example such thermostats could be set to a lower temperature in the winter time and the mobile thermostats could enhance and increase the temperature or temperatures in desired locations based on but not limited to, time of day or night, location, proximity to humans and animals, plants, fish, etc., motion or heat detected from living creatures including but not limited to humans and/or animals, voice commands, voice recognition, gesturing, alarms, remotely sent commands, outside temperature, distance and expected time of arrival, etc. In some embodiments of the present invention, the conventional, non-programmable, or non-remotely programmable fixed location thermostats could be augmented by/with a wired, wired and/or powerline controller located at and connected electrically to the furnace(s), central air conditioner(s), humidifier, dehumidifier, HVAC system(s), combinations of these, etc. which is able to either work with or take control from the existing conventional, non-programmable, or non-remotely programmable fixed location thermostats to control, set, monitor, etc. the temperature, humidity, air flow, etc. Embodiments of such a wired, wireless and/or powerline controller can be in parallel with or in series with or be implemented to take over control and block control of the conventional, non-programmable, or non-remotely programmable fixed location thermostats. As an example this can be accomplished by connecting the wired, wireless and/or powerline controller to the standard wiring for and between the conventional, non-programmable, or non-remotely programmable fixed location thermostats., for example, the furnace(s), air conditioners, central air conditioners, HVAC, humidifier(s), dehumidifier(s), combinations of these, etc. and having the wired, wireless, and/or powerline including combinations of these controller directly wired into the standard wiring for the furnace(s), air conditioners, central air conditioners, HVAC, humidifier(s), dehumidifier(s), combinations of these, etc.

The present invention can coordinate with other storage/charging systems including wall mount, desk furniture (which may also have, for example, solar cells attached to, for example, but not limited to sofas, chairs, seats, tables of any type, desks, shelves, ottomans, love seats, beds, head boards, dressers, pianos, foot boards, end tables, bookshelves, floors, walls, ceilings, etc. so as to be able to optimize/maximize the energy storage and usage. For example if the storage devices/units (e.g. batteries) that are being charged by the solar cell shades reaches full capacity, the present invention can open or partially open the solar cell shades and/or redirect the solar energy to other sources of energy harvesting including solar heat or solar cells so as to increase and distribute the energy storage and/or usage by allowing light to power and continue to power and provide energy and/or to use the excess energy to power heaters, fans, coolers, air conditioners, televisions, DVD, DVR and/or Blu-ray players and recorders, washers, dryers, stoves, furnaces, lighting including solid state lighting, combinations of these, etc.

The one or more main thermostat(s) may be replaced with a wireless or wired transceiver or receiver and/or transmitter that uses very little energy and may or may not have a display and connected and interfaced either wirelessly or wired (or both) to a remote sensor or sensor which could, for example, be a smart phone, tablet, computer, sensor (which may or may not have a display), etc. to effectively relocate either temporarily or permanently the effective location of the temperature setting element of the thermostat(s). Any or all (or a subset) of the thermostat(s), “mobile thermostat(s)”, etc. may also receive power or be power assisted from sources such as solar cells/photovoltaic cells and elements, RF, vibrational, mechanical, acoustical, inductively coupled, etc. In addition, embodiments of the present invention may employ wireless power transfer including, but not limited to, inductively coupled, resonant coupled, etc. energy/power transfer including from the solar cell shades to batteries, cell phones, tablets, smart phones, computers, lights, fans, heaters, coolers, air conditioners, phones, stereos, televisions, DVD, DVR, Blu-ray, other forms of media and entertainment, etc.

The present invention can be used to monitor any and all features, parameters, conditions, mood, settings, environment, electrical, optical, temperature, etc. information and store any and all information including color settings, color+white settings, combinations, color settings, color plus white settings with other temperature, humidity, moisture, audio, visual, sensory, vibration, mechanical, electrical, optical information, data, parameters, etc. Such storage can be of any type including, but not limited to local, mobile based device, cellular phone based, tablet based, remote control based, web based, cloud based, etc. Such stored information can be shared and transferred to others including, but not limited to, other mobile based device, cellular phone based, tablet based, remote control based, web based, cloud based, etc.

The power source for the present invention can be any suitable power source including but not limited to linear regulators and/or switching power supplies and regulators, transformers, including, but not limited to, forward converters, flyback converters, buck-boost, buck, boost, boost-buck, cuk, inverters, convertors, chargers, distribution etc. The present invention is not limited to the choices discussed above and any suitable circuit, topology, design, implementation, method, approach, etc. may be used with the present invention. Other power handling circuits can be incorporated in the home automation system, including those disclosed in U.S. patent application Ser. No. 13/674,072, filed Nov. 11, 2012 for a “Dimmable LED Driver with Multiple Power Sources”, and in U.S. patent application Ser. No. 13/301,457, filed Nov. 21, 2011 for a “Fluorescent Replacement”, which are incorporated herein by reference for all purposes.

The types of choices, selections, options, etc. for the present invention can be displayed automatically, manually, or by any other method, way, approach, implementation, etc. For example, these can be selected via physical commands, methods, and ways, such as, but not limited to, touching, typing, moving, speaking, tones, including tone of voice, using a mouse or cursor, pen, etc., vibration, light, etc.

The present invention can also use applications (Apps) either specifically or generally designed for the particular mobile device such as an iPhone, Android phone, Android tablet, iPad, iPod, etc. The present invention can also allow manual and/or automatic firmware and software upgrades to, for example, the mobile device applications, if any, and the controller that interfaces with solar shades/drapes/shutters/curtains/blinds, HVAC and also the lighting sources, internal controllers. Embodiments of the present invention can be also monitor, log, store, etc. the states and conditions system including but not limited to the dimming level including the dimming level and/or power level reduction or increase of heaters, fans, blowers, coolers, etc., the color combinations/selections/levels/ etc., the on-off status and state, the power level, the efficiency, the power factor, the input and output current, voltage and power, etc.

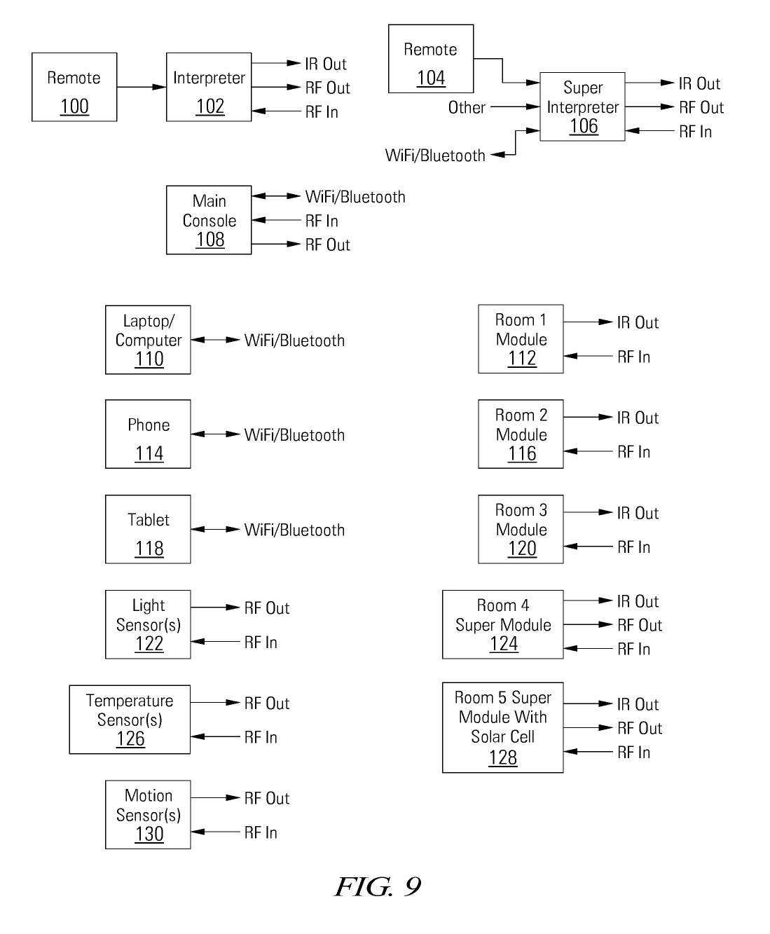

Turning to FIG. 9, a home automation system with mobile sensors is depicted in block diagram form in accordance with some embodiments of the invention. A remote control 100 of any type can transmit commands to (and, in some embodiments, receive status information back from) an interpreter 102, which interprets commands from the remote control 100 and which forwards the commands to other appropriate elements of the home automation system in whatever suitable format and transmission medium is used by the destination, such as, but not limited to, IR or RF output commands. In some cases, the interpreter 102 can also receive RF input from other devices, for example to control the interpreter 102 or to provide status information to be forwarded to the remote 100. Remotes (e.g., 104) can also transmit commands to (and, in some embodiments, receive status information back from) a super interpreter 106, which interprets commands from the remote control 104 and which forwards the commands to other appropriate elements of the home automation system in whatever suitable format and transmission medium is used by the destination, such as, but not limited to, IR or RF output commands, WiFi/Bluetooth or other format output commands. In some cases, the super interpreter 106 can also receive RF input from other devices or information or control signals from other sources, for example to control the interpreter 106 or to provide status information to be forwarded to the remote 104. In some cases, the super interpreter 106 can interact with and control other interpreters (e.g., 102) in the system.

A laptop or other computer 110 can interact with the home automation system with any wired or wireless connection, including but not limited to a WiFi or Bluetooth connection, in order to control one or more devices in the system or to receive and display status information. Similarly, a phone 114 and/or tablet 118 can interact with the home automation system with any wired or wireless connection, including but not limited to a WiFi or Bluetooth connection, in order to control one or more devices in the system or to receive and display status information. The home automation system can include one or more input devices such as, but not limited to, light sensor(s) 122, temperature sensor(s) 126, motion sensor(s) 130, etc., each of which can be connected to other elements of the system by RF links, IR links, WiFi or Bluetooth, or any other wired or wireless connections. Such information of all types including but not limited to control codes, preferred settings, scheduling and sequencing, time of day and date, other information discussed herein, etc. can be stored, for example in any or all or a subset of the system, modules, supermodules, subsystems, elements, implementation, embodiments, etc. of the present invention.

One or more room modules (e.g., 112, 116, 120) can be provided and placed in rooms having devices to be controlled, such as, but not limited to, heating or cooling devices, lighting systems, entertainment systems or audiovisual equipment such as televisions, receivers, speakers, media players, audio players, etc., and can be connected to other elements of the system by RF links, IR links, WiFi or Bluetooth, or any other wired or wireless connections. Room super modules (e.g., 124) can include all of the functionality of room modules (e.g., 112, 116, 120) and can also coordinate or control other modules (e.g., 112, 116, 120) to coordinate including but not limited to scheduling and sequencing the control of heating or cooling devices, lighting systems, entertainment systems or audiovisual equipment such as televisions, receivers, speakers, media players, audio players, etc. across multiple rooms or throughout a house or other building. Power can be provided to elements in the system from any of a number of sources, including wall power, battery power, or solar power (e.g., in a room super module with solar cell 128), including solar power from solar cells mounted directly on the elements or drawn from solar curtains/blinds/shades etc. or from storage devices charged by solar curtains/blinds/shades etc.

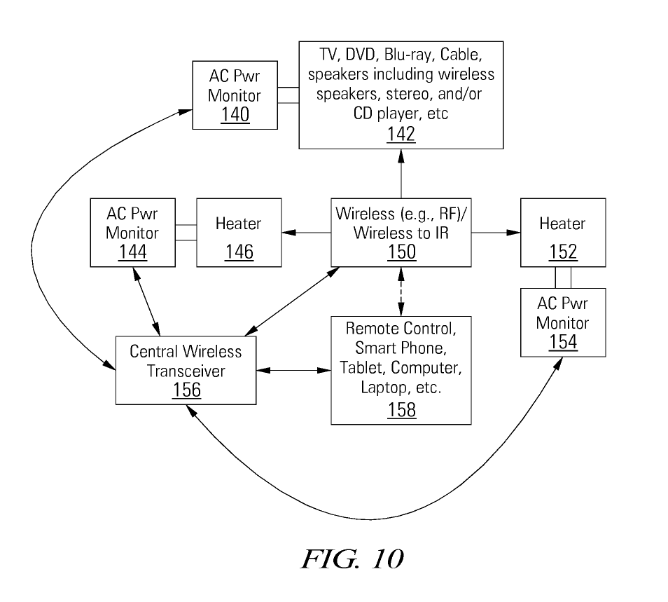

Turning now to FIG. 10, a block diagram depicts wireless/wired connections between components of a home automation system with mobile sensors in accordance with some embodiments of the invention. One or more heaters (e.g., 146, 152), coolers or HVAC equipment can be controlled by wireless controllers or interpreters (e.g., 150). Other elements (e.g., 142) of the home automation system such as, but not limited to, lighting systems, entertainment systems or audiovisual equipment such as televisions, receivers, speakers, media players, audio players, communications systems, etc. can also be controlled by wireless controllers or interpreters (e.g., 150). Power monitors 140, 144, 154 can monitor and/or control the power provided to the heaters (e.g., 146, 152) and/or other elements (e.g., 142) of the system, receiving control commands and/or sending status or other monitoring information to one or more central wireless transceivers 156. One or more remote controls, smart phones, tablets, computers, laptops etc. (e.g., 158) can also interface with the system, for example by wireless connection to central wireless transceivers 156 and/or by optional wireless RF or IR links to interpreters (e.g., 150).

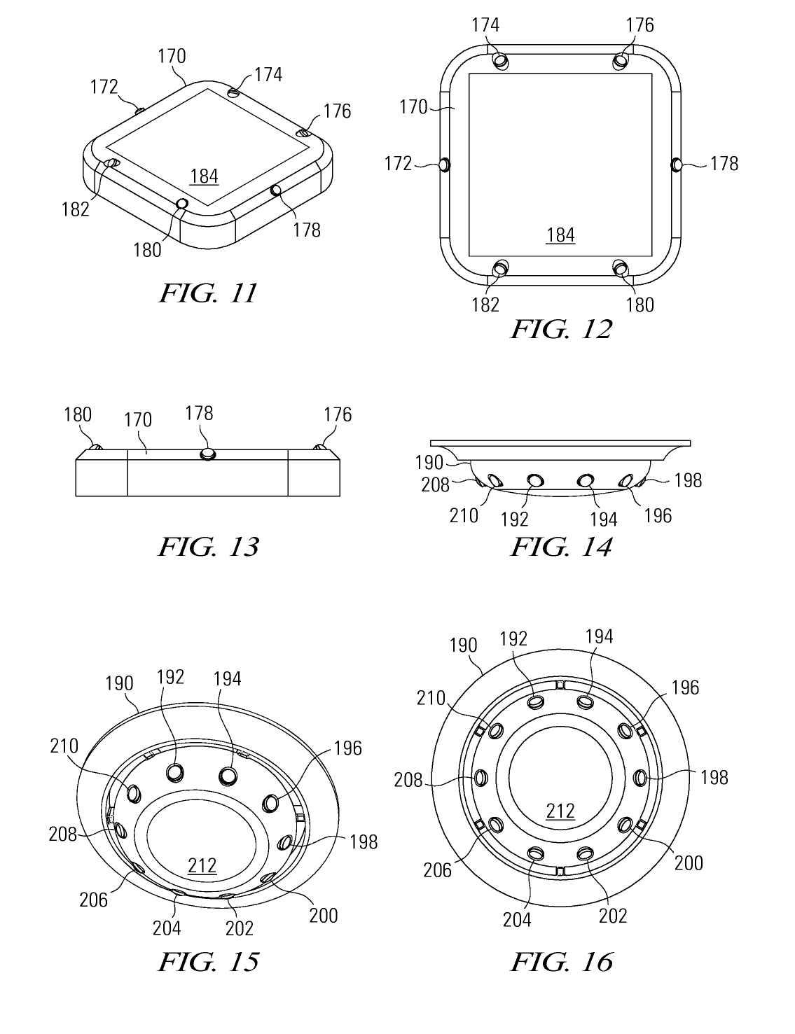

Turning now to FIGS. 11-13, an IR sensor 170 and/or transmitter suitable for use in some embodiments of a home automation system is depicted in perspective, top, and side views in accordance with some embodiments of the invention. The number of IR LEDs in the IR sensor 170 can be one or more. The IR sensor 170 can include multiple IR sensors and/or emitters 172, 174, 176, 178, 180, 182, to provide the desired direction(s) and sensitivity of coverage. The IR sensor 170 can consist of IR sensor arrays and can have any suitable shape, such as but not limited to the box of FIGS. 11-13 or the circular shape of the IR sensor 190 of FIGS. 14-16. In FIGS. 14-16, an IR sensor 190 and/or transmitter suitable for use in some embodiments of a home automation system is depicted in side, perspective, and top views in accordance with some embodiments of the invention. The IR sensor 190 can include multiple IR sensors and/or emitters 192, 194, 196, 198, 200, 202, 204, 206, 208, 210, 212 to provide the desired direction(s) and sensitivity of coverage and movement, rotation, angle(s), tilt(s), etc. Indicators such as LED lights can also be provided on IR sensors to indicate when commands are received and/or transmitted. The IR sensors (e.g., 170, 190) may include a solid state light (SSL) (e.g., 184, 212) including OLEDs, LEDs, SSLs and/or QD lights that could be room, task, general, etc. white light, white changing, color changing, etc., combinations of these, etc. The IR sensors 170,190 enable IR controlled only heater(s), air conditioner(s), fan(s), all other device, units, HVAC, entertainment, TVs, discussed herein, etc. units to be included in the home automation system, where such devices can only be used with remote controls typically which are hand held and can be made to be part of a simple to sophisticated wireless connected system using the present invention.

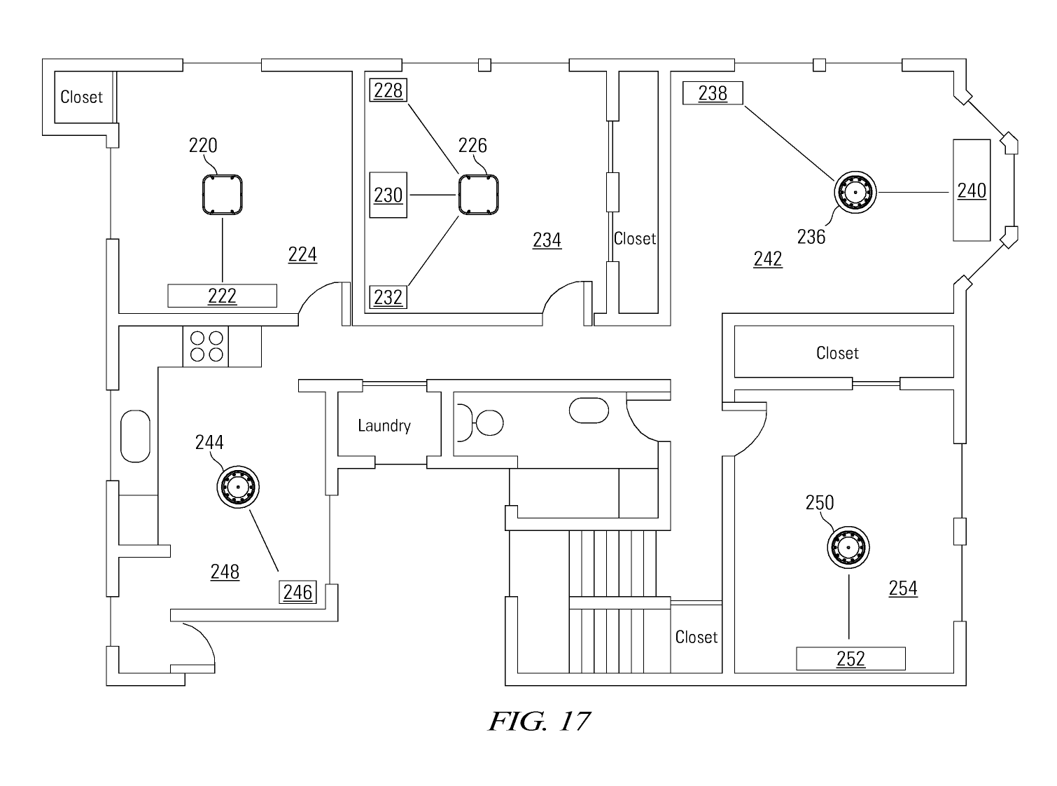

Turning to FIG. 17, an example home floorplan illustrates usage of an example embodiment of a home automation system in accordance with some embodiments of the invention. However, it is important to note that the floorplan and the usage of elements of the home automation system are merely non-limiting examples. An IR interpreter 220 is used to transmit commands to a television 222 in a bedroom 224. Another IR interpreter 226 is placed in a multimedia room 234 to control a receiver 230 and speakers 228, 232. An IR interpreter 236 is placed in a living room 242 to control a remote heater 238 and television 240. An IR interpreter 244 is placed in a kitchen 248 to interface with/receive settings from a thermostat 246, whether fixed/hardwired in place or remote, such as a temperature measurement device and/or temperature setting control interface in a smart phone or other remote control device. An IR interpreter 250 is placed in a bedroom 254 to control a television 252. Notably, other devices can be controlled in any or all rooms, and other control devices such as, but not limited to, a central wireless transceiver and/or super interpreter and/or main console and/or room super module can be included to coordinate status and control of any or all of the devices including the lighting and temperatures in the system.

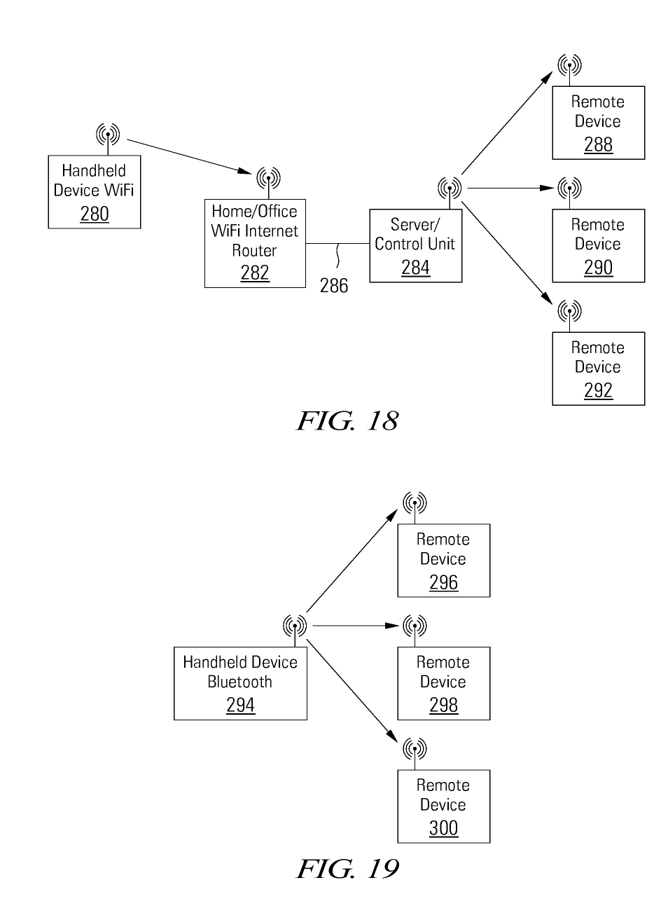

Turning to FIG. 18, a diagram depicts WiFi connections in an example embodiment of a home automation system in accordance with some embodiments of the invention. A server/control unit 284 can be connected by WiFi or other connections to remote devices 288, 290, 292 such as, but not limited to, heaters, coolers or HVAC equipment, lighting systems, entertainment systems or audiovisual equipment such as, but not limited to, televisions, receivers, speakers, media players, audio players, communications systems, power supplies and/or power controls/monitors, etc. Handheld devices, mobile thermostats, remote controls, etc. (e.g., 280) can be connected to the server/control unit 284 by wired/wireless connections or combinations thereof, for example through a home/office WiFi Internet router 282.

Turning to FIG. 19, a diagram depicts Bluetooth connections in an example embodiment of a home automation system in accordance with some embodiments of the invention. A handheld device, mobile thermostat, remote control, etc. (e.g., 294) can be connected by wireless connections such as, but not limited to, Bluetooth connections to remote devices (e.g., 296, 298, 300) such as, but not limited to, heaters, coolers or HVAC equipment, lighting systems, entertainment systems or audiovisual equipment such as, but not limited to, televisions, receivers, speakers, media players, audio players, communications systems, power supplies and/or power controls/monitors, etc.

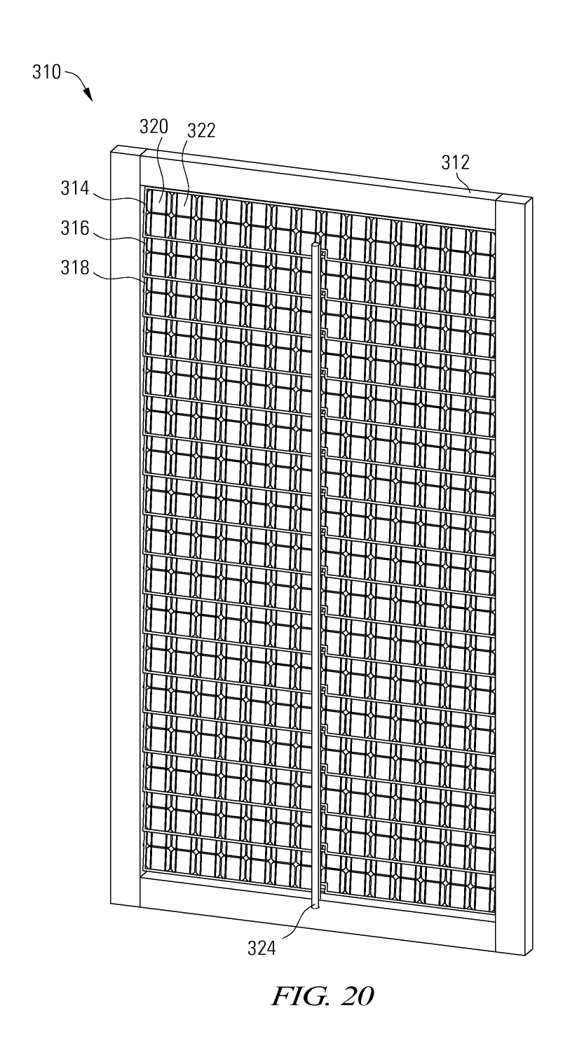

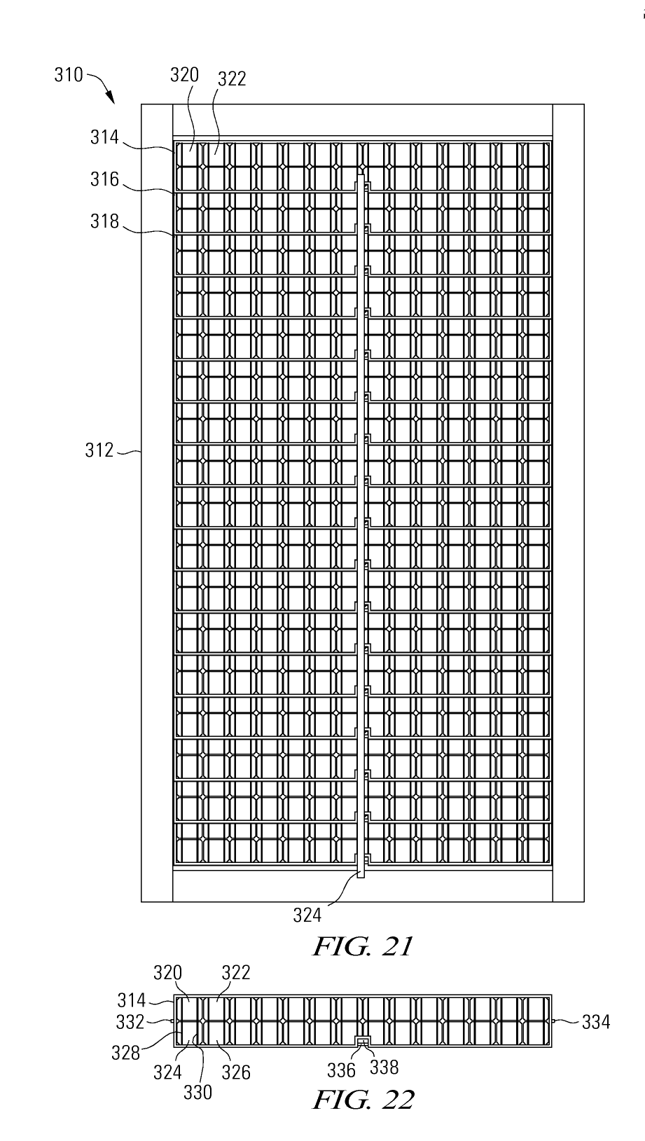

Turning to FIG. 20, a plantation shutter window covering 310 having solar collection louvers is depicted in perspective view in accordance with some embodiments of the invention. Each louver (e.g., 314, 316, 318) in the frame 312 includes a number of solar cells/panels (e.g., 320, 322) and can be rotated to expose or hide the window pane, as well as to orient the solar cells/panels (e.g., 320, 322) toward sunlight, for example using a manual control arm 324 to rotate the louvers (e.g., 314, 316, 318). The plantation shutter window covering 310 is depicted in front view in FIG. 21, and a single solar collection louver 314 is depicted in front view in FIG. 22. Electrical connections (e.g., 328, 330) between solar cells/panels (e.g., 320, 322, 324, 326) can, for example, be connected through the louver frame to connection points 332, 334, by which the solar collection louver 314 is rotatably connected in the plantation shutter window covering 310, allowing electrical current to be gathered from each louver (e.g., 314) in the plantation shutter window covering 310. A rod 336 and notch 338 can be used to connect a manual control rod (e.g., 324) in some embodiments.

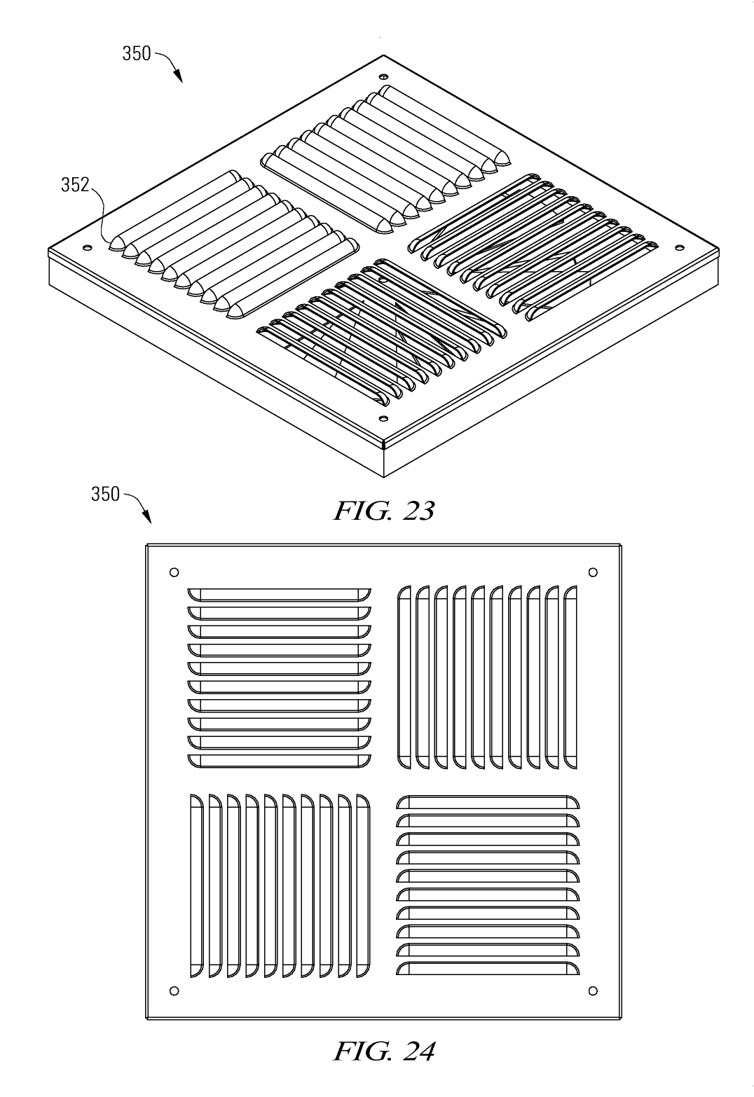

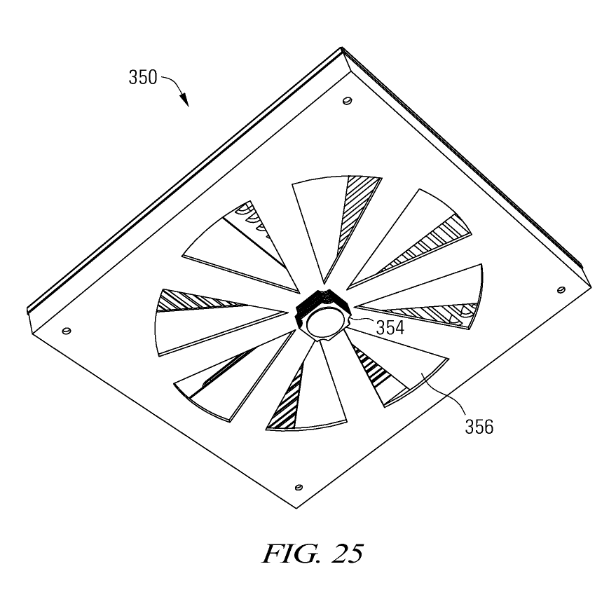

Turning to FIGS. 23-27, an automated register assembly 350 suitable for use in some embodiments of a home automation system is depicted in accordance with some embodiments of the invention. The automated register assembly 350 is shown in perspective top view in FIG. 23, top view in FIG. 24, perspective bottom view in FIG. 25, and bottom views in FIGS. 26 and 27 with airflow control blades (e.g., 356) in substantially open and closed positions, respectively. Air from, for example, HVAC ductwork is forced through the vent openings (e.g., 352) when a blower is active. A motor 354 such as, but not limited to, a stepper motor, DC motor, solenoid and gear system, etc. is connected to airflow control blades (e.g., 356) to open and close the airflow path through the vent openings (e.g., 352) under immediate or scheduled control from remote control devices, computers, laptops, tablets, smart phones, etc.

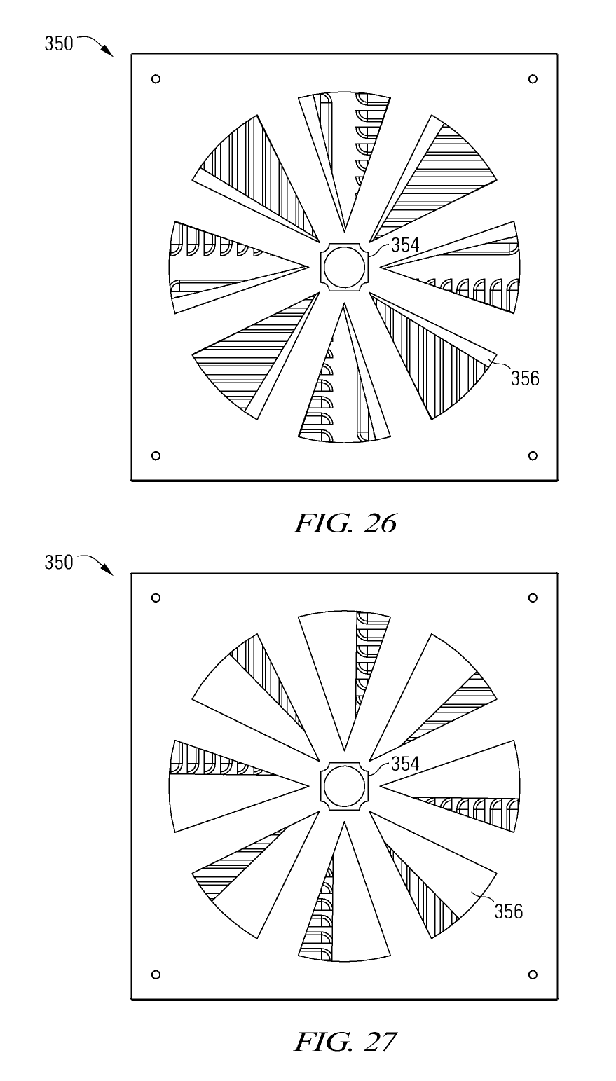

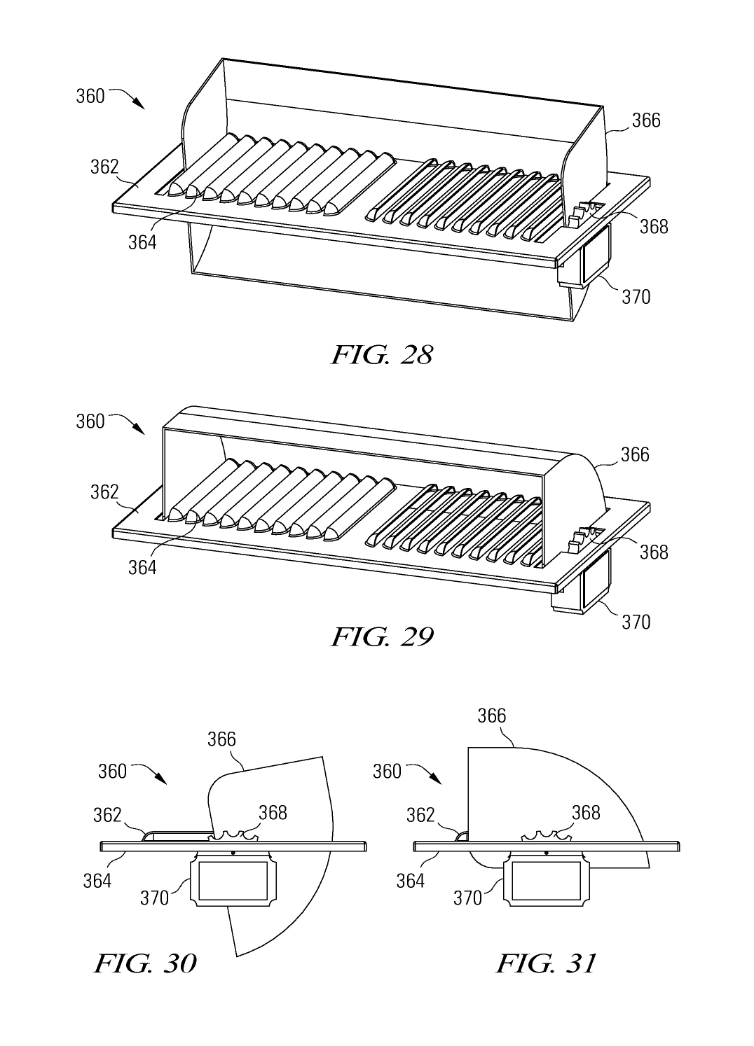

Turning to FIGS. 28-31, an automated register assembly 360 with a motorized directional control in accordance with some embodiments of the invention is depicted in perspective view in FIGS. 28-29 and side view in FIGS. 30-31, in open position in FIGS. 28, 30 and in closed position in FIGS. 29, 31. Air from, for example, HVAC ductwork is forced through the vent openings (e.g., 364) when a blower is active. A motor 370 such as, but not limited to, a stepper motor, DC motor, solenoid and gear system, etc. is connected to an airflow direction control hood 366 to allow air to flow substantially upward or to redirect the air in another direction, under immediate or scheduled and/or sequenced control from remote control devices, computers, laptops, tablets, smart phones, etc. A manual control wheel 368 can also be provided in some embodiments.

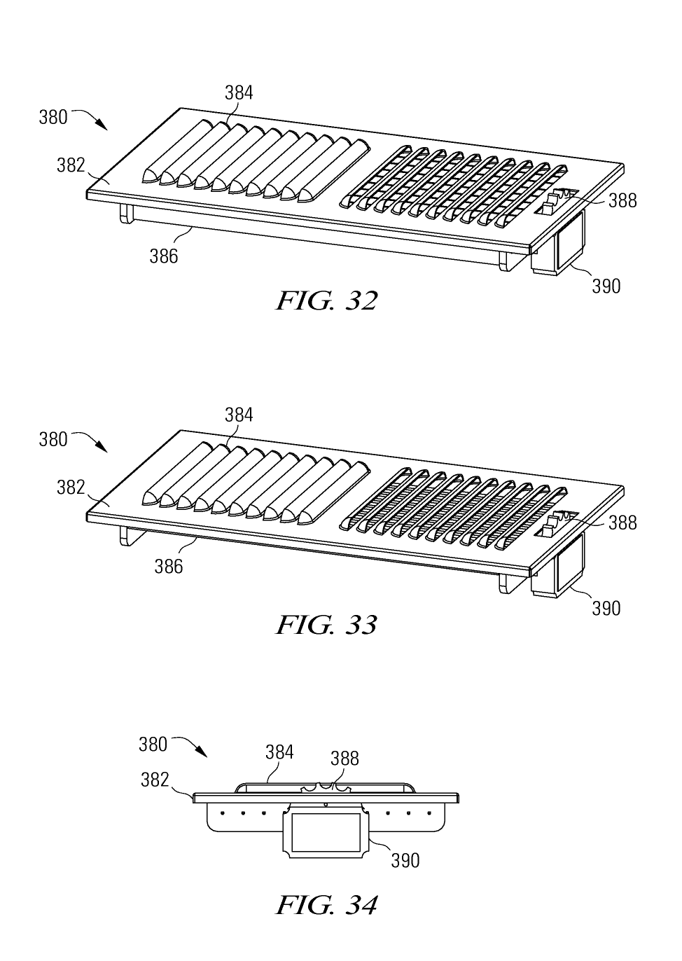

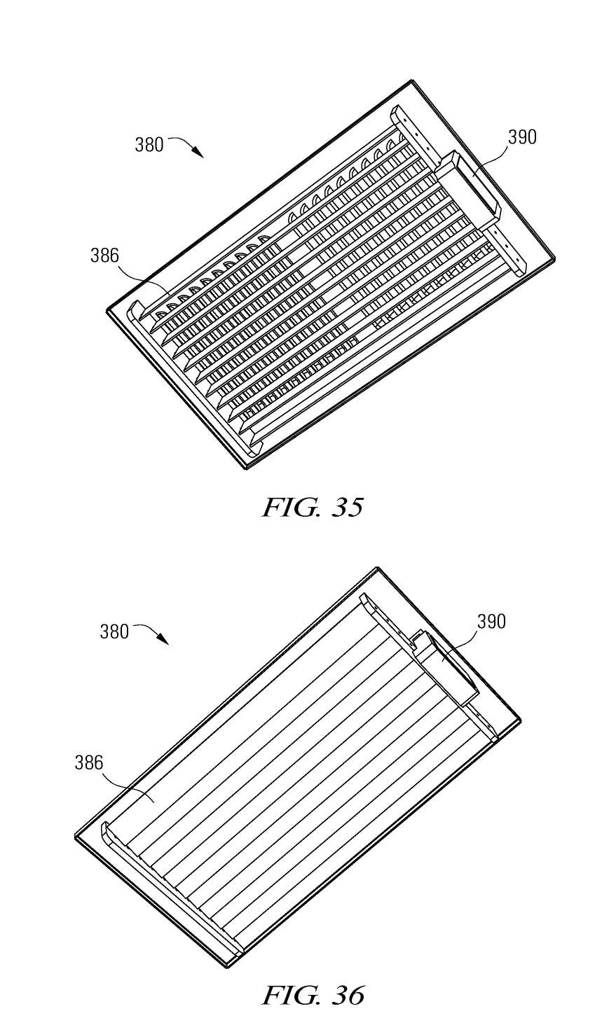

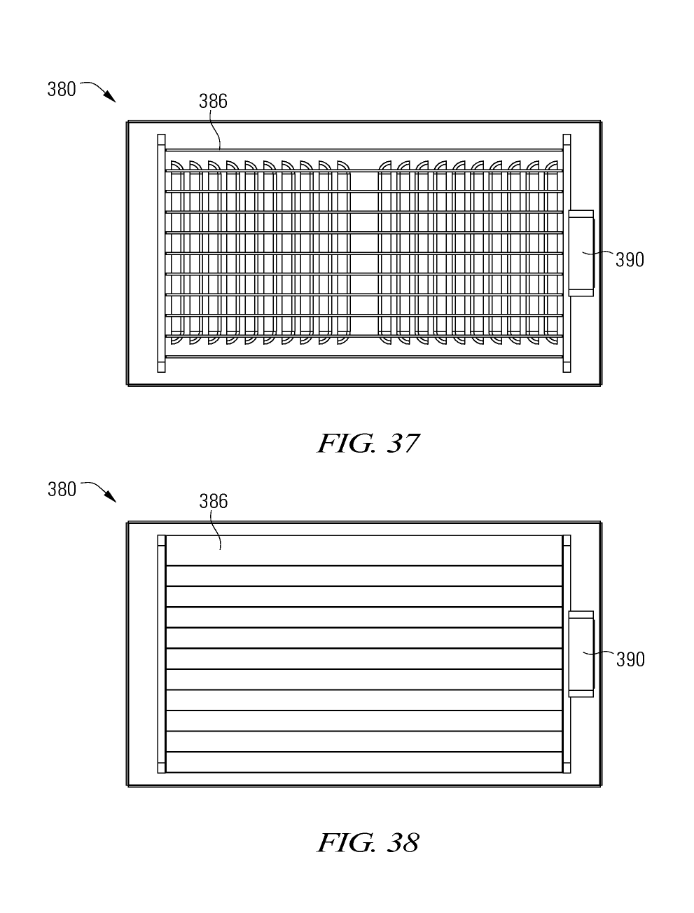

Turning to FIGS. 32-38, another automated register assembly 380 suitable for use in some embodiments of a home automation system is depicted in accordance with some embodiments of the invention. The automated register assembly 380 is shown open in top perspective view in FIG. 32, closed in top perspective view in FIG. 33, in side view in FIG. 34, open in bottom perspective view in FIG. 35, closed in bottom perspective view in FIG. 36, open in bottom view in FIG. 37, and closed in bottom view in FIG. 38. Air from, for example, HVAC ductwork is forced through the vent openings (e.g., 384) in the register body 382 when a blower is active. A motor 390 such as, but not limited to, a stepper motor, DC motor, solenoid and gear system, etc. is connected to rotatable blades (e.g., 386) to block or pass air, under immediate or scheduled control from remote control devices, computers, laptops, tablets, smart phones, etc. A manual control wheel 388 can also be provided in some embodiments.

The present invention can support all standards and conventions for 0 to 10 V dimming or other dimming techniques including but not limited to DALI, DMX, BACNET, other building systems standards. In addition the present invention can support, for example, but not limited to, overcurrent, undervoltage, overvoltage, short circuit, under current, and over-temperature protection. Interfaces that support standards including Building Automation Control Network (BACnet) developed as an open, standard communication protocol by the American Society of Heating, Refrigerating, and Air-Conditioning Engineers (ASHRAE) and LON (LonTalk), a protocol developed by the Echelon Corporation later named as standard EIA-709.1 by the Electronics Industries Alliance (EIA) that have been established for building automation system (BAS) vendors, manufacturers, suppliers, etc. can also be implemented in embodiments and implementations of the present invention including but not limited to in residential and commercial buildings and, for example associated automation. A purported primary feature of BACnet and LON is interoperability which should allow multiple control systems and lighting systems manufactured by different vendors (including the proposed system for this DOE SBIR) to work together, sharing information via a common interface which can be included in embodiments and implementations of the present invention.

The invention can support all types of lighting solutions including LEDs, CFLs, incandescent, halogen, xenon, HID and other light sources including other SSLs for the purpose of but not limited to providing light in emergency situations such as lighting that directs towards building exits, providing emergency light for critical operations, or any other uses where light is required for emergency or non-emergency needs.

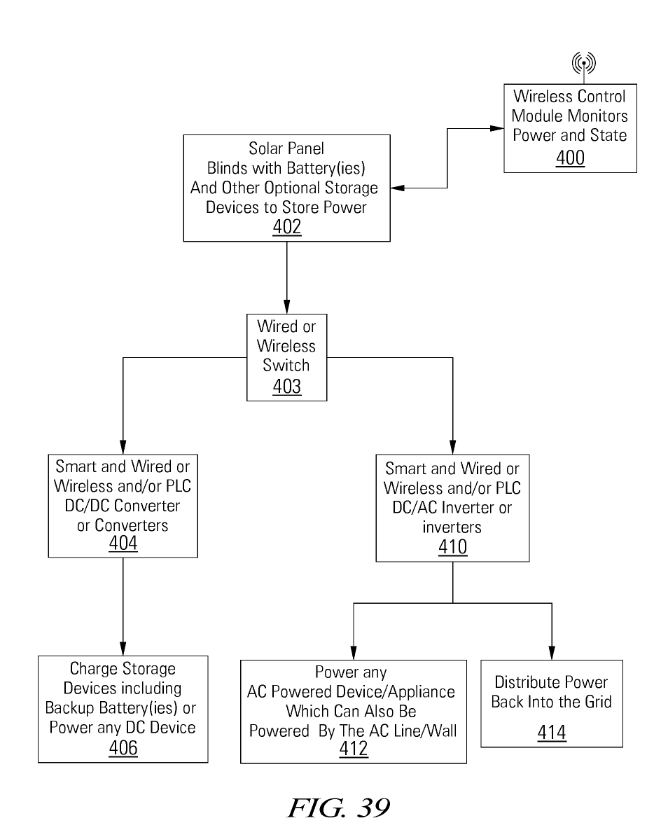

Turning to FIG. 39, wireless monitoring of power conversion and usage for collection and storage of power from solar window coverings is depicted in accordance with some embodiments of the invention. A wireless control module 400 monitors power and state of power usage in the system, including but not limited to monitoring solar panel blinds with battery backup 402. Power from the solar panel blinds with battery backup 402 can be switched between components in a wired or wireless switch 403 and converted to desired voltage and/or current levels in a DC/DC converter 404 and used to charge a backup battery or to power any DC device 406. Power from the solar panel blinds with battery backup 402 can also be converted to desired voltage and/or current levels in a DC/AC converter 410 and used to power any AC device 412 or distributed into the power grid 414. In some embodiments, the wireless control module 400 can also be used to control switches throughout the system, controlling power to DC and/or AC devices to turn devices on and off, and where appropriate, to control power levels, for example to control dimming levels and/or color of lighting systems.

The energy stored from the solar panels can be stored in a battery that can be used to charge personal devices during the daytime and also overnight such as, cell-phones, tablets, other batteries, or any other hand-held device or electronic device. This will eliminate the need for power plugs that are used to charge these devices and will reduce energy use in the household. Using a DC/AC inverter, AC appliances can be powered from the battery to potentially heat/cool rooms, power lights at night, televisions, audio/visual players, etc. A battery can be charged and taken with the individual to charge their individual hand held devices throughout the day as a backup battery.

DC to AC inverters and DC to DC converters including both smart converters and inverters as well as smart distributed DC to AC inverters and DC to DC converters can be used with the present invention. The electronics for converters, inverters (i.e., DC to AC), chargers, distribution, etc. in general may use and be configured in continuous conduction mode (CCM), critical conduction mode (CRM), discontinuous conduction mode (DCM), resonant conduction modes, etc., with any type of circuit topology including but not limited to buck, boost, buck-boost, boost-buck, cuk, SEPIC, flyback, forward-converters, etc. The present invention works with both isolated and non-isolated designs including, but not limited to, buck, boost-buck, buck-boost, boost, flyback and forward-converters. The present invention itself may also be non-isolated or isolated, for example using a tagalong inductor or transformer winding or other isolating techniques, including, but not limited to, transformers including signal, gate, isolation, etc. transformers, optoisolators, optocouplers, etc.

The present invention may include other implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, digital signal processors (DSPs), application specific integrated circuits (ASICs), etc. can also be used either alone or in combinations including analog and digital combinations for the present invention. Parts of the present invention can be incorporated into an integrated circuit, be an integrated circuit, etc.

The present invention may be used with a linear regulator, a switching regulator, a linear power supply, a switching power supply, multiple linear and switching regulator and power supplies, hybrid linear and switching regulators, hybrids of these, combinations of these, etc.

The present invention can also incorporate at an appropriate location or locations one or more thermistors (i.e., either of a negative temperature coefficient [NTC] or a positive temperature coefficient [PTC]) to provide temperature-based load current limiting.

The present invention can also be used for purposes and applications other than discussed above.

In general electrical heating where a heating element or elements are electrically controlled to, for example, maintain the temperature at a location at a certain value can be employed with the present invention. The present invention can also include circuit breakers including solid state circuit breakers and other devices, circuits, systems, etc. that limit or trip in the event of an overload condition/situation. The present invention can also include, for example analog or digital controls including but not limited to wired (i.e., 0 to 10 V, RS 232, RS485, IEEE standards, SPI, I2C, other serial and parallel standards and interfaces, UARTS in general, etc.), wireless, powerline, powerline communications (PLC),etc. and can be implemented in any part of the circuit for the present invention.

Embodiments of the present invention can use comparators, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices, field programmable gate arrays, etc.

The present invention includes implementations that contain various control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc.

The example embodiments disclosed herein illustrate certain features of the present invention and not limiting in any way, form or function of present invention. The present invention is, likewise, not limited in materials choices including semiconductor materials such as, but not limited to, silicon (Si), silicon carbide (SiC), silicon on insulator (SOI), other silicon combination and alloys such as silicon germanium (SiGe), etc., diamond, graphene, gallium nitride (GaN) and GaN-based materials, gallium arsenide (GaAs) and GaAs-based materials, etc. The present invention can include any type of switching elements including, but not limited to, field effect transistors (FETs) of any type such as metal oxide semiconductor field effect transistors (MOSFETs) including either p-channel or n-channel MOSFETs of any type, junction field effect transistors (JFETs) of any type, metal emitter semiconductor field effect transistors, etc. again, either p-channel or n-channel or both, bipolar junction transistors (BJTs) again, either NPN or PNP or both, heterojunction bipolar transistors (HBTs) of any type, high electron mobility transistors (HEMTs) of any type, unijunction transistors of any type, modulation doped field effect transistors (MODFETs) of any type, etc., again, in general, n-channel or p-channel or both, vacuum tubes including diodes, triodes, tetrodes, pentodes, etc. and any other type of switch, light/optical/temperature/humidity/pressure/force/position sensing device, circuit, system, etc. solar cells including all types and makes or photovoltaic, hybrid, etc. solar cells.

The present invention can use fans and simple to complex HVAC vent shutters with solar, batteries, other energy harvesting, wireless energy transfer, etc. to provide power to these.

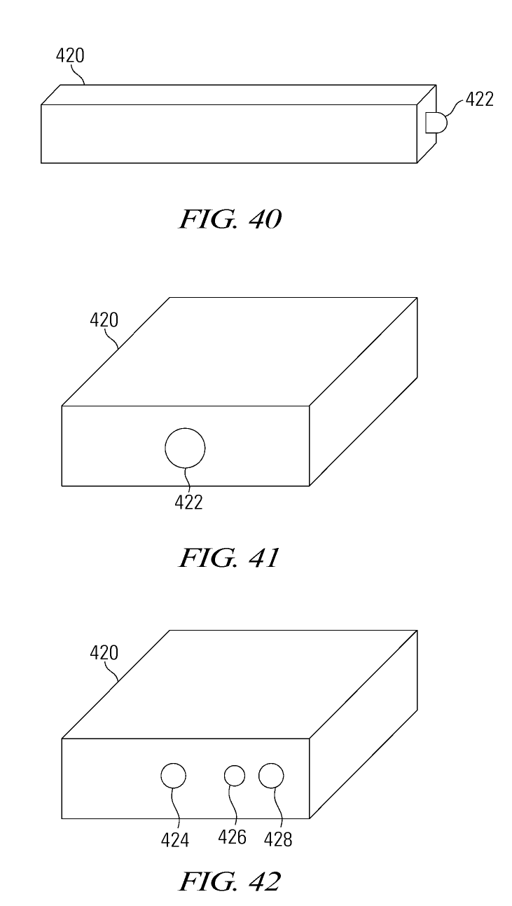

The system network can be comprised of wireless signals and command data that can be transmitted and received via a server or main control unit which can be connected to a wireless network where it can be utilized and controlled by smart phones, tablets, personal device, computers, etc. These commands and communications can be controlled and managed through software applications designed on platforms like, but not limited to, Android, iOS, C++, and Java using a graphical user interface (GUI). This main control unit can be used to communicate data and commands to and from the shades, curtains, drapes, blinds, etc. This system is also able to communicate commands to devices that control infrared devices such as, heaters, air-conditioners, televisions, personal fans, air purifiers, DVD units, DVR units, cable boxes, etc. These devices can also capture commands from remote controls that use infrared LEDs to control their counterparts. An example IR interpreter 420 that can capture commands from IR remote controls, interpret if needed, and forward in any suitable format including in IR form, is depicted in side, front perspective and rear perspective views in FIGS. 40-42 in accordance with some embodiments of the invention. An example IR interpreter 420 can include, for example, an infrared LED 422, phototransistor 422, transmit (TX) indicator 424, and power indicator 426. The information can be stored anywhere and everywhere including but not limited to the supermodules, the modules, smart phones, tablets, laptops, servers, cloud-based and web-based, etc. for certain embodiments of the present invention.

The commands for these various infrared controlled devices can be stored on, for example but not limited to, flash micro-chips, for example, inside the remote devices that are wireless extensions of the main control unit or server. The main control unit or server can have capabilities of handling Internet Protocol (IP), Transmission Control Protocol (TCP), and User Datagram Protocol (UDP) network information from the user's smart phone, tablet, personal device, computer, etc. This main control unit or server then outputs commands or data through a wireless protocol to the remote units which will then respond to the commands sent by transmitting infrared light to the devices or perform tasks such as switching a device on/off or other various tasks. Another way of controlling the remote devices in a wireless manner is using the Bluetooth protocol. This will allow the user to connect to the remote devices directly from the smart phone, tablet, personal device, computer, etc., without having to connect to the main control unit or server. It is also possible to connect the server to the remote devices via the power lines that already exist in the home or business. These remote devices can interact with a user without using a main control unit or server. The remote device can sense when a user enters a room by using Bluetooth to recognize the user by the Bluetooth unique universal identifier (UUID) and adjust the shades, curtains, drapes, blinds, heaters, air-conditioners, televisions, personal fans, air purifiers, DVD units, DVR units, Blue-ray, cable boxes, etc., to the user's liking based on personal settings on their device containing that contains a Bluetooth radio. In addition RFID can also be used in place of or in conjunction with, coupled, etc. with the Bluetooth, WiFi, etc.

These remote devices can transmit information back to the user. Information such as battery voltage, current usage, power, state, power level, efficiency, temperature, duration, humidity, input current, input voltage, input power, output current, output voltage, output power, etc. They have the ability to transmit and receive data in order to carry out their programmed functions. This data will be presented on the user's device through, for example, a GUI and managed by the software application running on the user's device. These remote devices allow the user to control a multitude of devices, as mentioned previously, in their home or business. The software applications also allow the user the ability to program automated functions into their remote devices to, for example, control and automate the lighting or heater/air-conditioner when the user is away from the home or business and to combine the functions of more than one remote control on to customized remote pages where, for example, one selection can turn on (or off) multiple devices at essentially the same time or in sequence including, for example, but not limited to, heater(s), lighting, television(s), satellite receivers, cable receivers, stereos, radios, CD players and recorders, other devices, units, etc. discussed herein, other audio-visual devices, units, appliances, etc. Having the device connected to the Internet through an Internet router, the user can control their remote devices on the wide area network (WAN) from outside their home or business network when the user is away. In embodiments of the present invention, alarms, proximity, global positioning system (GPS) information, location, signals, etc. can be used. The present invention can be present to turn on or off or dim or increase lighting, heating, cooling, temperature, air flow, other appliances, etc. home entertainment including but not limited to satellite receivers, DVD players and/or recorders, DVRs, cable boxes, stereos, TVs, etc. In some embodiments of the present invention light panels may be included and inserted on the interior facing side of the drapes, curtains, shades, blinds, etc. Customized smart phone, tablet pages that allow multiple device settings to be grouped together for entertainment including, for example, but not limited to entertainment and HVAC settings such as television, DVD, DVR, cable, heaters, fans, room and other lights including but not limited to, air conditioners, etc. on the same web page/screen, etc.

The present invention can relay information about temperature, light intensity and quality to HVAC installations that use, for example, heat pumps and compressor type AC units to intelligently control the percentage use of the two or more units working in parallel. For example, heat pumps work efficiently only in a limited temperature range and need to be supplemented outside this range. The present invention can control and instruct multiple HVAC components to work independently or in synchrony to maximize efficiency and reduce energy usage.

The present invention will be capable of interfacing to commercial and non-commercial systems in which energy may be produced and sold back to the electrical grid including residential or commercial/industrial energy systems comprising of photovoltaic solar cell installations, fuel cell energy storage, wind turbines, and any other energy system(s), renewable or otherwise, that require analyzing for home consumption as well as when the energy is provided to the grid. The invention is also capable of itself providing energy that may be sold back to the community electrical grid.

The afore mentioned appliances and devices can also be controlled or triggered by a motion sensor or proximity based sensor using the present invention on the remote device. For example, with the present invention, when an individual walks into a room, the motion sensor can turn on the heater, lights, open/close blinds, etc. This can be done by sensing motion or reading a RFID tag or ID that would be on the person or on their phone or personal device. Using a RFID tag will help make the room appliance settings personalized to the individual upon entering a room and/or building. The remote sensing devices such as an RFID reader can log when an individual is in a room to a computer or record the frequency and duration the RFID tag or ID entered/exited the room and/or building. Accelerometers can sense if the individual or person is in a laying, sitting, or standing position. This application can be applied in a home, business, hospital, care taking facility, etc., for monitoring individuals. The remote device can, for example, alert the server, main controller, or personal devices such as phones, computers, tablets, machines, appliances, health practitioner, etc., if an individual suddenly fell or was in a laying or sitting or other position for too long of a period of time.

Embodiments of the remote device can detect water, moisture, water leaks, flooding, etc., to turn on a pump or alert emergency services, phones, tablets, computers, etc., to prevent damage to homes, businesses, or other buildings/structures. It can be used to detect moisture in lawns at homes, businesses, golf courses, schools, etc., and water facilities when moisture is not detected and to prevent watering lawns and facilities when rain is present. These remote devices can also be used to sense humidity levels in rooms to maintain the humidity levels and keep them constant. They can also track data about the humidity levels and when they change. This data can be transmitted to the server or main control unit or to other devices such as phones, tablets, computers, appliances, machines, etc. It can also be used in wells to monitor ground water levels wirelessly and transmit data back to computers or to users for monitoring. Large crop farmers can use the remote devices for watering crops with the correct amount of water by sensing the amount of moisture or water in the soil. It can also use this data to automatically control the amount of water that is distributed to the crops and soil and water parts of the crop that are drier than others.

The remote device can also detect temperature to prevent fires or fire damage. They can alert emergency services, individuals, computers, phones, tablets, machines, appliances, etc., when there is a fire or smoke present in the building or structure. It can trigger the sprinkler or fire suppressant system to put fires out in a specific room or area to prevent flooding and water damage to the rest of the building and structure. They can be used to detect smoke, chemicals, and/or gases to alert emergency services or open ventilation systems to clear warehouses, labs, hospitals, homes, businesses, etc., or to trigger systems that prevent smoke, chemicals, and/or gases from becoming too concentrated or dangerous or to alert systems, machines, phones, tablets, computers, etc., that smoke, chemicals, and/or gases are present.

The remote device can be used to monitor voltage, current, and power in batteries that store power from solar panels. They can log this information and transmit it to devices that can display this data on a GUI for a user to monitor the status of the battery and solar panel or other ways to view the information including by e-mail, text, numbers, alphanumeric characters, etc. They can also connect to a DC/AC inverter to monitor the power output to devices and switch them on and off according to the amount of power stored into the battery that is being charged from the solar panels. This remote device can control the appliances or devices that are connected to it via AC connection. For example, a heater can be plugged into the DC/AC inverter and the device can control the when the user decides. This heater can be powered from the battery that was charged from the solar panel and also from the AC power lines which can be switch selectable including user or utility selectable. The user can control the heater, monitor the battery or batteries or other storage source(s) power, current, voltage level(s) from the solar panel, and customize on and off times based on month, day, and time data supplied by the user, utilities and/or other sources. This control system is not limited to a heating, cooling, flowing, etc. or other appliance(s).

The user can schedule events and/or tasks for heaters, fans, coolers, air conditioners, central air, televisions, DVD, DVR, Blu-ray, cable and satellite boxes and service providers, other appliances and/or devices to turn on/off and specific times using software applications on their phone, tablet, computer, personal device(s), etc., by specifying the month, day, hour, and minute when a device can turn on/off or perform any other varying task. For example, if the user is not present in a home, residence, apartment, condo, building or structure the lights can be scheduled to turn on to imitate the idea that the building is occupied or to turn them off to save the energy cost on the power bill. Scheduling and tasks can be prioritized and sequenced. Embodiments of the present invention also allows for priorities to be adjusted, reevaluated, be conditional, etc. The remote device can also be controlled using a personal electronic device for use by mentally or physically impaired individuals. It will allow them the ability to control devices such as, fans, heaters, air-conditioners, lights, televisions, audio/visual players, home appliances, etc., without having to physically turn them on or change their settings. These devices can also be controlled when the individual is not present in the room or building remotely or scheduled and/or sequenced events and/or tasks can be set on the users electronic device and other parts, components, modules, submodules, etc. which will trigger specific events at the designated times. If the individual is not able to program specific scheduled and/or sequenced events, another user can specify these events for them. This way the impaired individual will have appliances or devices and, for example, temperature, humidity, lighting, color temperature, etc. controlled automatically. For example, at a specific time in the evening the blinds, drapes, shades, curtains, shutters, etc. will close and the lights would come on for the individual who is unable to do those things alone.

The remote device can also be used to monitor properties, homes, businesses, etc., for security purposes. Motion sensors can be used to detect motion or magnetic sensors to detect doors or windows opening and closing. The motion and/or proximity detectors and sensors can be used to collectively determine the path of a person, animal, other entities, including an intruder. For example, either external (i.e., outdoors) or internal (i.e., indoors) motion and/or proximity detectors and/or sensors may be used to collectively determine, evaluate, decide, respond to, alarm, turn on lights, turn on alarms, make noise, voice emulate words, provide recorded messages, turn on/off lights, strobe lights, sequence lights or other devices, appliances, HVAC, etc. These devices can alert phones, tablets, computers, emergency services, etc., when these devices sensors are triggered. They can also turn on lights or trigger other devices in and around properties, homes, businesses, etc., when motion is detected or doors or windows are opened. The scheduling and sequencing can range from simple to complex, from one command for one, for example, heater, cooler, air conditioner, HVAC, TVs, DVDs, DVRs, Blu-ray, cable and/or satellite providers interface boxes, other devices and appliances, etc. The sequencing and scheduling can also be dependent on existing, future, alternative, etc. events, conditions, scenarios, etc.

Lighting may be controlled, dimmed, selected, monitored by wireless (including but not limited to Bluetooth, WiFi, ISM, IEEE 801, 2.4 GHz, etc.) or wired (DMX, DALI, RS 232, RS 485, serial, SPI, U2C, USB, etc.) means by the home automation system.

Smart T8, T5, T12, CFL, other fluorescent lamps types, etc., E26, E27, A-lamp, MR-16, GU-10, PAR 30, PAR 38, R 30, 2×2, 2×4, 2 ft.×2 ft., 2 ft.×4 ft., 1 ft.×3 ft., 3 ft.×1 ft., ½ ft.×2 ft., ½ ft. by 4 ft., etc. panels, smaller, larger custom, other sizes, sizes to fit into existing luminaires and fixtures, etc., down light, can light, under cabinet, over cabinet, sconce, troffer, pendant fixtures, chandelier fixtures, under cabinet, over cabinet, track lighting, etc. Lighting panels used or powered in the invention can include waveguided, edge emitting, edge lit, back lit, direct lit, directly lit, surface lit, surface emitter, and edge emitter, combinations of these, etc. LED lighting and lighting panels, etc. and combinations of these. The lighting panels can be white, RGB, RGBW, RGBA, RGBAW, etc., combinations of these, etc.

If the power is too high for the heat sink in lighting, the home automation system can limit then cut back the power. To determine/set/evaluate limit, can calculate or use temperature sensor(s), thermistors thermocouples (TCs), positive coefficient thermistors, negative coefficient thermistors, IC temperature measurement, semiconductor temperature measurement, etc.

The present invention works with all types of ballasts including instant start, rapid start, programmed start, dimmable ballasts, etc. Embodiments of the present invention can have internal or external power supplies/drivers.

Should the ballast at some future time fail to work properly, fail to operate, stop working, etc., the present invention allows the ballast to be disconnected, removed, etc. and, for example, a new ballast or a new power supply, power source, to be used with the present invention such that the new power source could be connected to the input of the external driver or to directly to the LED and/or OLED lights, lamps, lighting, etc. Embodiments and implementations of the external driver can have the capability to run off/be powered by AC line voltage in addition to being powered by a ballast. Embodiments and implementations of the present invention can automatically select between ballast and AC line voltage or manually, including a switch, or remote control to select whether to receive power from an AC line or a ballast (including an emergency power ballast).

In other embodiments of the present invention an input socket can be used to power the LED and/or OLED lights, lamps, lighting, etc. In other embodiments of the present invention an input and output socket can be used to power the LED and/or OLED lights, lamps, lighting, etc. such that unless power/current is applied to the input, the LED and/or OLED lights will not turn on.

The present invention can use a ballast as a power supply including but not limited to fluorescent lamp ballasts, high intensity discharge (HID) lamp ballasts, sodium lamp ballasts, etc. in which the power from the output of the ballast(s) can be used as a power source such as an AC or DC power source including where the power from multiple outputs of a single ballast or plurality of ballasts are combined. Embodiments of the present invention can use power combining with or without isolation of any type or form including but not limited to capacitors, transformers, inductors, diodes, resistors, transistors including but not limited to other components and devices and active devices including switches, transistors, triacs, thyristors, silicon controlled rectifiers (SCRs), synchronized transistors, integrated circuits (ICs), application specific integrated circuits (ASICs) of any type, any material, any material compositions including but not limited to heterojunctions, heteromaterials, etc. to provide and perform power combining of one or more ballast outputs. The power combined outputs can be single stage, two stage, multiple stage, etc. including, but not limited to, push-pull, forward converters, flyback, buck, buck-boost, boost-buck, boost, Cuk, SEPIC, half-bridge, full-bridge, voltage mode, current mode, current fed, voltage fed, etc.

In some embodiments of the present invention, the current/power of one or more lamp outputs may be combined in any number of ways including multiple ways of providing power to individual direct fluorescent lamp replacements including the example embodiment of the present invention using power combiners, power combining, etc.

Embodiments of the present invention can work with instant start, programmed start, and/or rapid start compatible. An IC or ICs can be or can include, contain, be part of, etc., a microcontroller, a microprocessor, a field programmable gate array (FPGA), an ASIC, multiple chips including being assembled and packaged together or separately that perform these functions that may also include one or more wireless and/or wired interfaces to communicate and control, monitor, dim, etc. the present devices. In some embodiments of the present invention, for example, the fluorescent lamps comprise one or more panel lights that can fit into, be interfaced with, be connected to, be retrofitted, etc. using the existing ballast, connections, fixtures, etc.

Embodiments of the present invention can be used with different fixtures and can allow additional features not currently possible including having colors such as RGB, RGBA, other color combinations, one or more colors, white plus colors, full spectrum, form factor change other than T8, T12, other fluorescent lamp shapes, etc. including changing to, for example but not limited to, approximately 2 ft.×2 ft., 3 ft.×2 ft., 3 ft.×3 ft., 2 ft.×4 ft., 3 ft.×4 ft., etc.

The present invention can also be used to provide a smart, intelligent and interactive light source to treat seasonal affective disorder (SAD) among other light/phototherapy treatments/applications/needs/ etc. For example, the present invention can be used to aid in SAD treatment by turning on appropriate brightness, color temperature, wavelength(s), intensity, light output lighting at one or more locations within a room, house, building, hospital, care facility, nursing home, anti-depressant facility or location, work environment, business, industrial setting, locations, etc. Such SAD treatment lighting can be put on the back (i.e., facing inside/interior) of solar curtains, solar drapes, solar shades, solar blinds, solar panels, etc. and coordinated, scheduled and/or sequenced with the solar energy/power uses of the present invention including harvesting energy to be used a later time to power the SAD treatment lighting, or to time shift the lighting or to perform other scheduled events including being used to simulate a sun rise wake up by gently or otherwise (e.g., quickly, immediately, ramped from zero (full dimming) to full intensity/power/lumens/etc. over a prescribed amount of time that can set or programmed by the user, automatically, by caregivers, by family or friends, by others, by the season and time, date, etc. of the year, remotely, locally, etc.). In a similar fashion, the present invention can be used to simulate sunset at any time of the day in any location in the world including locations with long periods of sun hours or short sun hours (e.g. Alaska, Nordic countries, parts of the world close to the North Pole, South Pole, etc.) depending on things such as the time of the year, weather, altitude, shadowing, obstructions, enhancement of light due to reflections including reflections off of surfaces, etc. In addition, circadian rhythms enhancements, alignments, resets, adjustment, shifts, etc. may also be accomplished and embodied in the present invention. For example, but not limited to, a person or persons who need to work night shifts including late night shifts may use the present invention to shift their effective and localized sunrise and sunset by, for example, using the solar shades, solar blinds, solar curtains, solar drapes, solar panels, etc. to block and absorb sunlight including sunrise in the morning and store that energy while providing, for example, a dark, completely dark or nearly dark, etc. environment to aid and promote healthy sleep including the production of appropriate melatonin during the daytime while storing the Sun/solar/other light/EM energy to be used at an appropriate wake-up time even if that wake up time occurs at dusk or dark and then use the stored energy to provide appropriate levels and intensity illumination including artificial illumination from solid state lighting, fluorescent lighting and other sources of lighting to simulate and stimulate, for example, but not limited to, full spectrum lighting, partial spectrum lighting, blue wavelength/shifted lighting, red wavelength/shifted lighting. The lighting can also be coordinated, scheduled and/or sequenced with heating or cooling of the room, location, environment as well as turning on (or off) radios, televisions, cell phones, computers, tablets, personal digital assistants (PDAs), other entertainment and/or communications devices, systems, components, etc. Embodiments of the present invention can accomplish this by many methods including but not limited to receiving signals from one or more sensors and detectors including, but not limited to wired and wireless signals, feedback, information, etc. from one or more devices including time, day and date information, global positioning system (GPS) information, weather conditions, atomic clock signals and information, solar sensors and detectors, sunlight sensors and detectors, photo sensors and detectors, light sensors and detectors, electromagnetic and/or optical detectors, frequency and/or wavelength detectors and sensors, CCD imaging including visible and/or infrared imaging, sensing and detection, infrared detection and sensing, ultraviolet detection and sensing, spectrum analysis, detecting and sensing, optical and electromagnetic spectrum detection and sensing, temperature sensors and detectors, humidity sensors and detectors, barometric sensors and detectors, rain and/or snow sensors and detectors, moisture sensors and detectors, wind sensors and detectors, other location and proximity sensors and detectors, motion sensors and detectors, etc. and/or combinations of these, etc. These and other types of information, sensors and detectors may also be combined and/or connected with wearable devices and other sensors that can detect, for example, but not limited to, heart rate, blood pressure, phase of the circadian rhythm cycle, other information about circadian rhythm, ambient light, pressure, movement, electroencephalogram/electroencephalography (EEG), electrocardiography/ electrocardiogram (EKG or ECG), brain waves, oxygen level, brain waves, muscle movement, body temperature, pulse rate, actimetry, sleep actigraphs, temperature, polysomnography (PSG), mood, emotional state, etc. Wearable devices can include, but are not limited to, wrist devices, or watch-shaped devices worn on the wrist of the non-dominant arm, detectors and sensors, sleep management and monitoring sensors, systems, etc. including for awake, REM, deep sleep, various other states of sleep and wake, etc., delayed sleep phase disorder, perspiration, orientation, location, vertical or horizontal sensing, etc., speech, speech patterns, voice, weather, etc. Such signals, input, feedback, information, etc. can be used to, for example, to set the level, spectrum and intensity, emulated sunlight spectrum, white temperature, color temperature, duration and intensity of treatment, etc. In addition, sensors can include light sensors, photosensors, spectrum analyzers including optical spectrum analyzers, light sensors with notch filters, motion sensors, proximity sensors, radio frequency identification (RFID), cell phones, smart phones, tablets, etc. Smart phones, tablets, laptops, computers, dedicated control and/or interface units, etc. may be used to, for example, but not limited to, transmit and/or process the information via applications or apps or can use apps to display, store, log, analyze, etc. data, results, performance, control, provide feedback, etc. The present invention can incorporate and use open platforms including but not limited to Google Fit, Apple HealthKit, FitBit, etc. The present invention allows for scheduling/programming of events remotely including for persons who are unable to do so themselves which can also include remote scheduling, programming, monitoring, control, etc. The present invention can also be used to treat and/or assist in the treatment of dementia and related conditions. The present invention can also provide power for other uses, functions including but not limited to fans, motors, heaters, blowers, fan blades, security cameras, surveillance cameras, monitors, monitoring systems, web-based cameras, motorized cameras, etc., USB and other charging, auxiliary power, etc., battery backup, emergency batteries, microphones, speakers, sensors, WiFi, wireless power, combinations of these, etc. In some embodiments of the present invention, various wireless approaches can be used that for example, but are not limited to, involve WiFi and Bluetooth to communicate with devices including but not limited to smart phones, iPods, iPads, iPhones, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention. In some embodiments of the present invention, a buck, buck-boost, boost-buck, and/or boost switching topology is used to provide power for the present invention. As an example, a buck circuit can be used to provide AC to DC regulated power to the present invention. An example of an efficient way of providing such power is to for example have the buck circuit be controlled based on the lowest and strictest required regulation voltage that typically is used for the control circuits such as, for example, the integrated circuits which could, for example, consist of but is not limited to a microcontroller, microprocessor, FPGA, DSP, CLD, etc., one or more of these or each of these, wireless or wired ICs, interfaces, devices, protocols, etc. including but not limited to, WiFi, Bluetooth, IEEE 801, ISM frequencies, other bands and frequencies, I2C, RS232, RS485, DMX, DALI, SPI, USB, serial, etc., combinations of these including one or more of the same or different ones, etc. that is used with one or more windings (as discussed in U.S. patent application Ser. No. 13/674,072, filed Jun. 2, 2013 for a “Dimmable LED Driver with Multiple Power Sources” which is incorporated herein by reference for all purposes) on the buck inductor to provide multiple outputs including, for example, but not limited to, typically 3 V to 5 V for the control electronics, 5 V to 15 V to 20 V for the power devices including the gate drive for the power transistors including FETs and in some embodiments bipolar junction transistors (BJTs) and Darlingtons and IGBTs. In addition to these windings, a winding or windings for, for example, can also be used to provide power to the LEDs and/or OLEDs as well as power for other needs and applications including fans, motors, USB, battery chargers, etc. Linear regulation, linear regulators, switching regulators, voltage regulators, current regulation, current regulators, shunt, regulation, shunt regulators, combinations of these, etc. may be used.

In some embodiments of the present invention persons and, for example, animals experiencing or suffering from seasonal affective disorder and, for example, circadian rhythm and sleep disorders, etc. can also reap additional benefits that the present invention can have for these people and, for example animals, birds, other living creatures including people who sleep patterns are shifted, for example, at such as night shift workers, who often must sleep during the day and be awake at night or people recovering from jet lag, a change in time zones, countries, locations, daylight shifts, etc. that need to regulate their circadian rhythms and sleep patterns to that different from local day and night time.

Wearable fitness and/or monitoring devices can be interfaced with the system, including but not limited to Bluetooth wearable devices such as those from iFit, Nike, Fitbit, Withings, Apple, etc. For example, such devices can provide information about a person's condition that can be interpreted by the automation system to control lights, temperature, etc. in response. As a further example, lighting can be dimmed when a device detects that a person has fallen asleep, temperature can be increased when a device detects that a person is cold, etc.

As an example, the present invention could be set/programmed to close the solar shades, drapes, curtains, blinds, shutters, panels, etc. at a certain time of, in this example, evening or night, turn on certain lights of either certain wavelengths, color, color temperature, etc. set the temperature, humidity, then later turn off the lights either quickly or gently dim the lights down or completely off as the individual case may be, and either concurrently, nearly simultaneously, or sequentially including later sequentially turn of the radio and/or television and/or other entertainment or electronic, etc. devices, units, systems, etc. and adjust the temperature, humidity, etc. for the remainder of the night and, in the morning, either provide direct natural sunlight by raising the solar shades, drapes, curtains, blinds, shutters, panels, etc. at a certain time of the morning or turning on artificial lighting such as solid state lighting, fluorescent lighting, incandescent lighting, combinations of these, etc. along with setting temperatures in the same as well as optionally other locations which in some embodiments of the present invention are set by motion and proximity detectors and sensors, RFID, Bluetooth signal detection and strength, other RF, wireless, optical, infrared detection and sensing, etc., turning on televisions to a prescribed/set channel or AV source, turning on radios, alarms, strobes, etc. for a more abrupt and immediate wakeup or gently and slowly in continuous or step fashion turning on lighting of an appropriate wavelength, range of wavelengths, color, range of colors, color temperature, range of color temperatures, etc. Throughout the day additional coordination, sequencing, scheduling could take place, etc. some or all of which may be preprogrammed, automatic, or otherwise scheduled and some or, for that matter, all of which may be event based to trigger sequences of temperature and environment new settings including setting temperatures in one or more (certain) locations as well as humidity, lighting, entertainment choices, etc. The cycle could repeat itself exactly as the evening or night before or could be changed to be very different, slightly different, etc. based on a number of factors and inputs including but not limited to day of the week and/or date of the month/year, weather conditions, external or internal variables or parameters, work habits, conditions, requirements, etc., change in occupancy, visitors, friends and/or family visiting, health conditions, etc.

In another example, a person who works the late (graveyard) night shift may need to sleep in the morning. For such an example, the particulars may depend on the individual, however one scenario would be for the solar curtains, solar drapes, solar panels, solar blinds, solar shades, solar shutters, etc. to close at night and remain closed collecting whatever nighttime ambient light can be collected and then continue to collect solar light and day light from the Sun in the morning and until the person awakes in the afternoon or, for example, early evening. When the person returns home form the late night shift, the lighting in the house can be set to an intensity, level, color/wavelength range that is suitable and conducive with inducing and supporting sleep. The temperature and optionally other parameters such as humidity can be set to a comfortable and desirable level that is tailored for one or more personal comfort zones as well as appropriate background sounds including radios, CD players other sources of sound, music, voice, talk, etc. as well as, in some cases, television set to local stations, cable or satellite networks, etc. as well as, for example, heating up a favorite drink, snack and/or meal including in a totally automated, sequenced and scheduled fashion or partially or totally manually set and/or event detected and driven including, for example the person approaching (or leaving) the house, apartment, dwelling, location, residence, worksite, etc. All or some of these can and are powered by the energy stored via the solar shades, solar curtains, solar panels, solar shutters, solar blinds, solar panels, etc. which can also power the sources of the scheduling, detection and sensing, decision making, etc.

Using temperature sensors, this invention can relay information about rooms or parts of buildings with temperatures too extreme for people or animals to habitat, for example, when there is a fire present. This invention can alert individuals, set off alarms, alert personal devices, computers, emergency services, etc. It can also be used to sense when there is excessive moisture or water in a room or building to transmit alarms or alert users. For example, when a basement pipe breaks in the winter due to freezing, the sensor could sense changes in humidity and or sense water on the floor and alert the home owners or initialize a pump or other device. Another example, it can be used in large crop farming to detect rain fall, moisture in the soil, water table levels in a well, irrigation control and relay information about these sensors data wirelessly to a WiFi network and then to a user's smart-phone, tablet, computer, or other device to record data or control devices accordingly.

This invention can be used with motion sensors to trigger lights to illuminate rooms, buildings, the exterior of homes or businesses for security or other purposes. Motion sensors could trigger alarms or notifications on a user's smart-phone, tablet, or computer when there is movement in specific areas. The motion sensors could be used to open/close blinds or shutters when an individual enters a room in the morning/night to let light in or restrict light in specific rooms.

This invention can also aid in the case where individuals with disabilities that cannot perform basic tasks such as turning on appliances, televisions, audio and video players, opening blinds, switching lights, or operating heating and air-conditioning equipment, by giving them the ability to do this from touch screen devices or computers. This will help when individuals whom are not very mobile. With this invention it will be possible to control many devices in the home or building with a mobile electronic device or by setting up scheduled events within the software application running on the personal device. For individuals that have a difficult time operating a personal electronic device, the scheduling will help the individual operate appliances and electronics in the room or building without actually having to do it themselves. In the situation where there is not a smart-phone, tablet, computer or other personal device available, a custom remote can be used that will interact with the entire invention system that will allow the user to carry out the afore mentioned tasks remotely.

The invention can eliminate the need for multiple infrared remote controls for entertainment systems in homes and businesses by possessing the same commands as the devices in its flash memory. For example, the user can open up a software application on their smart-phone or tablet and operate every device within one application. This will eliminate the need for batteries and for the remote controls themselves in many cases. Again, the present invention allows for storage of commands and information in numerous locations including in multiple smart-phones, iPods, tablets, laptops, computers, servers, cloud and web-based storage as well as within the controllers, modules, supermodules and other components of the present invention.

The above examples are merely intended to provide simple descriptions of a small subset of the present invention and are in no way or form intended to be limiting in any manner. Any practical number of different and diverse events to a very large number can be evaluated, coordinated, scheduled, sequenced, executed, re-evaluated, adjusted, monitored, controlled, feedback, interpreted, etc. using the present invention including using the present invention with existing, relatively ‘dumb’ heaters, coolers, air conditioners, central air conditioners, humidifiers, dehumidifiers, appliances, entertainment centers including televisions, radios, stereos, cable TV, satellite TV, DVD, DVR, VHS, Blu-ray, other formats, CD, MP3 players, etc., appliances, combinations of these, etc.

Ballasts can be used as power sources and supplies with multiple uses, applications, voltages, power, current and voltage control, etc.

The present invention can be used to provide, control, dimming, on/off, monitoring, logging, decision making, etc. of providing power including wall power including in a single or dual wall plug or higher count in a single gang, two gang, multiple gang box size or as a plug-in extender, etc. The present invention can be wired, wireless, etc. The present invention can be mounted/installed in, for example but not limited to, in a standard wall outlet box, a wall dimmer, an on/off switch, a light socket, including but not limited to an A-lamp socket, a E26 socket, etc. The present invention can monitor, store, log, etc., electrical parameters including, but not limited to, current, voltage, power, power factor, apparent power, real power, AC current, DC current, AC voltage, DC voltage, etc. The present invention can select between dimming, dimming with on/off and on/off only by automatic, manual including switch(es), remote control, detection and analysis, etc. The present invention can, for example, measure the AC input voltage and produce a scaled version of the AC input voltage, measure the AC input current and produce a scaled version of the AC input current, measure any DC offsets to the input current, voltage, power, etc. measure the output current, voltage, power, etc. One embodiment for measuring the AC input voltage involves the use of high resistance resistors and one or more op amps. Such embodiments can involve level shifting if needed. Measuring either the input current or voltage or both can be accomplished by the use of op amps; for example, the current can be measured by measuring the voltage across a relatively low value resistance and then applied, and voltage shifted if needed, using an op amp or op amps. In some embodiments of the present invention, various wireless approaches can be used that for example, but are not limited to, involve WiFi and Bluetooth such that devices including but not limited to smart phones, ipods, ipads, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention.

The present invention can be used to provide assisted care or monitoring in general including using voice commands, voice recognition, image recognition, pattern recognition, wearable device(s) information, wired and wireless panic buttons, proximity sensors, motion sensors, sound sensors, etc. The present invention can take, use, analyze, make decisions, etc. based on data, signals, information, etc., from one or more sensors and detectors including, but not limited to wired and wireless signals, feedback, information, etc. from one or more devices including with wearable devices and other sensors that can detect, for example, but not limited to, heart rate, blood pressure, phase of the circadian rhythm cycle, EEG, EKG, oxygen level, brain waves, muscle movement, body temperature, pulse rate, mood, emotional state, location, GPS, elevation, sound, mechanical, movement, time duration, vibration, sound, pressure, accelerometer(s), sound spectrum, ultrasound, sonar, etc. Such signals, input, feedback, information, etc. can be used to, for example, to set the level, spectrum and intensity, emulated sunlight spectrum, white temperature, lighting sensors, duration and intensity of treatment, etc. In addition, infrared detectors and sensors, motion sensors, proximity sensors, RFID, cell phones, smart phones, tablets, etc. Smart phones, tablets, laptops, computers, dedicated control and/or interface units, etc. may be used to, for example, but not limited to, transmit and/or process the information via APPs or can use APPs to display, store, log, analyze, etc. data, results, performance, control, provide feedback, etc. The present invention can incorporate and use open platforms including but not limited to Google Fit, Apple HealthKit, etc. Telephone-based, Web-based, Cloud-based, etc., Cell phone based, combinations of these, etc. can be used to transmit, receive, communicate, recognize, alert, warn, contact, control, monitor, etc. In some embodiments of the present invention, various wireless approaches can be used that for example, but are not limited to, involve WiFi and Bluetooth such that devices including but not limited to smart phones, ipods, ipads, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention. The present invention can take a number of actions including flashing lights, contacting specified people, agencies, groups, services, departments, entities, individuals, etc. via web, mobile, smart, etc., cellular phones, tablets, other mobile devices, etc., land line, conventional phones, e-mails, text messages, cellular services, etc. In embodiments of the present invention, the absence of a signal, information, and/or response including but not limited to physiological including but not limited to blood pressure, heart rate, oxygen levels, insulin levels, temperature, other physiological monitors, sensors, etc., motion, proximity, temperature, humidity, room occupancy, room temperature, electrical power usage, lack of electrical power usage, water flow, water usage, gas usage, carbon monoxide and other gas sensing, lights and other appliances turned off or turned on (state of usage, time of usage, duration of usage), voice recognition, voice commands, sounds, movements, breakage, noise(s), patterns, etc.

The present invention can use linear regulation, switching regulation including but not limited to buck, buck-boost, boost-buck, boost, etc., transformer(s) with one or more secondaries, flyback(s) with one or more secondaries, switched capacitors, etc. The RS interface provides an appropriate emulation circuit or circuits for the heater/cathode connections of, for example, rapid start ballasts.

The present invention provides a direct replacement for fluorescent tubes used in ballasts and permits dimming even/including if the ballast is not designed to support dimming.

Both wireless and wired control, dimming and monitoring can be accomplished with the present invention. For example wired dimming using 0 to 10 V can be used or ISM, WiFi, Bluetooth, etc.

Use 0 to 10V other analog, DMX, DALI, RS232, RS422, RS485, USB, and other serial and/or parallel interfaces to communicate with the present invention. Use a connector or connectors to do so. Many embodiments will use an isolated interface.

Use, for example, but not limited to, a buck or boost or flyback or forward converter circuit that can be powered by AC lines (including universal voltage 80 to 305 VAC, 100 VAC, 120 VAC, 200 VAC, 220 VAC, 240 VAC, 277 VAC, 347 VAC, 480 VAC, etc. at, for example but not limited to, nominally 50/60 Hz) via, for example, but not limited to an EMI line filter that contains, for example, but not limited to inductors and which also can be powered by an electronic ballast that contains capacitors which limit/block/attenuate/etc. the 50/60 Hz line voltage and bypass (or put in parallel with, etc.) the EMI filter.

The present invention can be dimmable when powered on the AC lines or from the ballast.

The present invention can work with dimmable ballasts of any type including but not limited to 0 to 10 V, DALI, TRIAC, and powerline control (PLC), etc., instant-start ballasts, rapid start ballasts, programmed start ballasts, programmable start ballasts, pre-start ballasts, magnetic ballasts, and essentially any type of ballast.

The present invention can use a switch, including a momentary switch, for shock hazard protection. For example a momentary switch can be depressed to complete a circuit that allows the ballast to power the present invention once the momentary switch is released. Should a potential shock hazard exist the circuit would not latch and until the shock hazard is eliminated, pushing the momentary button would not latch and activate the circuit. The present invention can also use remote enable to provide protection including protection from shock hazard by essentially keeping the ballast turned off and in a high impedance state until remote commanded (i.e., by remote control, smart phone, tablet, computer, other device, user input, controls/buttons/etc. on the implementations, etc.) to disable the protection/shock hazard. In some preferred embodiments, the user will need to request to disable the protection/shock hazard and then verify/confirm that request to actually disable.

The present invention can use wireless control to control the dimming level of the lighting, etc.

The present invention allows for full spectrum, including full visible spectrum lighting and control, dimming and/or monitoring including red, green, blue (RGB); red, green, blue, amber, (RGBA); red, green, blue, white (RGBW), red, green, blue, amber, white (RGBAW), additional or fewer colors/wavelengths, etc., combinations of these, etc.

The present invention can use small cards, memories, etc. that can consist of any type of semiconductor memory, magnetic memory, ferromagnetic memory, optical memory, etc., including but not limited to FLASH memory, non-volatile memory, EEPROM, EPROM, PROM, AND memory, OR memory, etc. Such memory can be used to provide programmable information including, for example, but not limited to, name to be used for the present invention, address, individual address, group address, location, properties, behavior, pre-programmed features, data logging, storage of audio and or video information, etc., communications, encryption, type, security, etc.

The present invention, in addition to providing analog and/or digital interfaces for control (including dimming and monitoring, logging, etc.) can also provide isolated (or non-isolated) power derived from, for example, but not limited to, the ballast itself. An example would be to take current/power from the ballast by rectifying the AC output from the ballast and filtering as desired. Example embodiments which are not intended to be limiting in any way or form include using forward converters or flyback converters for isolated, using buck, boost, buck-boost, boost-buck, etc., linear regulators including current regulators, etc. In some embodiments of the present invention, a keep-alive circuit is used when the present invention is dimmed to very low levels or off. Non-isolated supplies can use isolated windings to provide isolation for example with buck-boost, buck, boost-buck, boost etc. topologies.

The present invention can work with all types of sensors and controls including ones that sense movement, proximity, light, solar light, solar energy, daylight, light spectrum(s), temperature, time of day, mechanical, electronic, electrical, sound, vibration, words, voice, voice commands, voice recognition, cell phones, smart phones, tablets, computers, servers, WiFi, Bluetooth, IEEE 802, ISM, USB, serial and/or parallel communications, RFID, entry cards, access cards, signal strength, etc. The present invention can also be used in simple and/or autonomous control and associated modes. Some implementations may require no external controller or a very simple, easy to use, intuitive one, etc. for the user to operate.

An example of an optical spectrum analyzer can consist of optical sensors and detectors that are wavelength/frequency/color specific and can be stacked either vertically (i.e., layered/stacked on top of each other) or horizontally (stacked side by side, etc.). Such detectors/sensors could be measured using current or voltage sensitive circuits that are fed or multiplexed to one or more analog to digital converters (ADCs) that can also be used to provide either analog or digital (or both) feedback/control/readout/etc. to/for the present invention. Such sensors/detectors can be arrayed or act separately/independently to control/feedback the intensity/color/wavelength/frequency levels, etc.

The present invention can also use wired and/or wireless interfaces including but not limited to serial interfaces including but not limited to those discussed herein to, for example, program/set/assign/etc., the address, name, identification, identifier, grouping, group, etc. Such setting/assignments/etc., can be also done/accomplished/performed by the user and be user-programmed, etc. The example serial port, for example, USB port can be used for other purposes including direct communications with the present device, reprogramming the parts (or all) of the firmware/software of the present invention, charging other devices using the example USB port, etc. Such other devices could include but are not limited to cell phones, smart phones, tablets, computers, batteries, other energy storage devices, other personal assistant devices, sensors and detectors, portable lighting, etc.

The present invention can be powered by a ballast in a number of ways including both magnetic and electronic ballasts including electronic ballasts that are instant-start., rapid start, programmable start, dimmable, etc. The ballast output(s) can be combined, connected, etc. as needed to achieve the needed/desired performance.

Some embodiments of the present invention can also be used to detect the presence (or absence) of a persons or persons including whether a person or persons are spending too much time or too little time in a particular location and, in some embodiments, automatically alert and provide alerts via, for example, but not limited to, e-mail, phone calls, web messages, text messages, etc.

The present invention can have current and/or voltage control or both including with automatic switchover from voltage to current control or current to voltage control. The setpoints, parameters, conditional statements, etc. be manually set, factory set, user set, remote control set using, for example, wired or wireless control, monitoring, communications, etc. The control can be local, fixed or remotely programmed and set. Wired control can include but is not limited to 0 to 10V, 1 to 8 V, 0 to 5 V, 0 to 3V, 0 to 10 V, etc., SPI, USB, powerline control, I2C, serial, SPC., etc. Wireless control can include but is not limited to ZigBee, Bluetooth, IEEE 802, WiFi, ISM, RF, IR, infrared, IrDA, infrared modulated control (i.e., 30 to 56 kHz), RFID, ZWave, etc.

In addition to the fans discussed herein, motorized track lighting and other lighting including but not limited to PAR, MR16, GU10, etc. may be used such that the motor(s) can be controlled locally and/or remotely. Such motors and related devices and components, etc. can also be used to tilt, move, extend, articulate, direct, swing out, etc. the lighting and, for example, the fans and other accessories, etc. Some embodiments of the present invention can also have DC to AC inverters that, for example, provide 50 or 60 Hz AC voltage (e.g., 120 V AC or 220 V AC) that can also be selected.

These examples of implementations of the present invention in which the fluorescent lamps have been replaced by embodiments and implementations of the present invention that, for example, consists of power supply or supplies that are powered by the ballast and provide conventional and new lighting designs, capabilities, form factors, etc. which can be both retrofitted into existing fixtures and luminaires as well, for example, into new construction, etc. The renderings are intended to be examples and in no way or form should be considered to be limiting of the present invention. The present invention can be supported in a number of ways including being supported by the electrical bi-pin connectors of the fixture or luminaire, by inserting other supports and structures, by using magnets, by using screws, tape, double sided tape, etc. The structures may also be illuminated by lighting including, but not limited to, OLEDs and/or LEDs which could be, for example, white, RGB, RGBW, RGBAW, etc. along with and, for example, various other types of functions and applications including those that provide/require electrical energy, mechanical strength, energy harvesting, solar detection, solar energy, daylighting harvesting, motion sensing, infrared sensing, spectrum sensing/detection, proximity detection/sensing, other sensors and detectors, etc. Note in some embodiments of the present invention the ballast bi-pins may be connected to the support structure so as to provide a path including an electrically safe path that allows electrical connection between the ballast and the power supply and the lighting that is powered by the power supply. This permits a number of advantageous features and functions including replacement/interchangeability of the power supply and/or the lighting, safe connections, shock hazard protection, etc.

As examples, a center tapped transformer, non-center tapped transformers and, in general, any type of transformer may be used including ones that require full bridge rectifiers, synchronous rectifiers, silicon controlled rectifiers, etc. In other embodiments, flyback transformer(s) may be used. Additional primary, secondary and other windings may be added/included/used/etc. in and with the present invention. For example but not limited to, power is fed to the lighting or additional circuitry, including for example, current control and/or voltage control, etc. The present invention can have shock protection, over current protection, over voltage protection, over temperature protection, etc. The present invention can use/have more than one color or more colors than just white or any color temperature of white including but not limited to, red green blue, red green blue amber, red green blue amber white, etc. and can provide current control (or, in some embodiments, voltage control or both) by shunting excess current from reaching the primary of the transformer. In these embodiments the AC output of the ballast provides the input for the transformer. As the electronic ballast typically puts out high frequencies often higher than 30 kHz to 40 kHz, the transformer(s) can be compact in size, weight, form factor, etc. The transistors, switches, etc. are configured in a back to back configuration with common gates and sources.

Lighting which can be bars of mostly any size and shape including but not limited to relatively long and thin ones such as nominally 1 ft.×4 ft. 1/2 ft. by 4 ft., 2 ft. by 4 ft., ½ ft.×3 ft., ½ ft.×3½ feet, etc. which can consist of lighting of one or more colors including virtually any color such as but not limited to, white, red, blue, green, amber, cyan, orange, violet, yellow, etc., other colors, combinations of these, etc. In some embodiments of the present invention, white light can be turned on to provide “Sun-like” illumination. In other embodiments of the present invention, the white light can be augmented with the color temperature set/controlled/modified by other colors including possible feedback with optical and/or spectral sensors/detectors/arrays/etc. In yet other embodiments of the present invention full spectrum Sun emulation can be accomplished by the proper selection of color light sources such as LEDs, OLEDs, quantum dots (QDs), combinations of these, etc. Embodiments of the present invention allow for protection against too much power to the various light source elements and colors to avoid, for example, degradation, damage and potential harm, etc.

T In addition, the fixture/luminare can also be used for support as well as surrounding fixtures, supports, ceiling, wall, floor, grids, etc. The present invention allows for turnable/twistable connections to, for example, the bi-pins of the fluorescent light sockets/fixtures that may also contain switches for a number of functions and purposes including but not limited to safety, shock hazard, ballast phase, ballast polarity, power requirements, power usage and selection, etc. In some embodiments of the present invention these switches may be automated, remotely selected, remotely controlled and monitored, etc.

The present invention does not only apply to fluorescent lamps and fixtures and luminares of all types and kinds—the present invention also applies in general to all types of high intensity discharge (HID) lighting including but not limited to mercury vapor lamps, metal-halide (MH) lamps, ceramic MH lamps, sodium-vapor lamps, xenon short-arc lamps, other types of arc lamps, sodium-based and other element-based lighting, gas discharge, etc.

Embodiments of the present invention can also have lighting on the outside of, for example, the light bar, panel, etc. including direct lit, edge lit, back lit, etc. Some example embodiments can also include one or multiple LEDs, OLEDs, QDs including examples herein that can consist of one or more of white, red, green, blue, amber, yellow, orange, etc. In addition, such lighting can be used to convey information about the status of a situation including flashing lights which may convey emergency situations, warning, greetings, alerts, alarms, attention, directions including changing colors or displaying shapes such as arrows, etc. The arrows can be different colors and point in different directions depending on the reason for the arrow to be displayed. As an example, a red arrow can point in the direction of an exit in case of an emergency including a flashing arrow. In other embodiments, for example, the arrow could flash or turn on and alternate with emergency lighting that could be white full brightness or dimmed down or another color or colors or be color-changing.

In other embodiments of the present invention wind energy/power harvesting may also be used independently or combined with the solar shades, blinds, covers, drapes, curtains, panels, etc. to increase, augment, offset, combine, etc. additional energy harvesting. Embodiments of the present invention allows for sharing of the energy storage elements, components and systems as well as the energy consuming components, devices, systems, etc. including but not limited to heaters, coolers, air conditioners, humidifiers, dehumidifiers, entertainment units including but not limited to televisions, cable, VHS, DVD, DVR and Blu-ray players and recorders, CD players and recorders, computers and laptops, tablets, other entertainment and audio-visual components, parts, systems, units, etc.

An example embodiment of the present invention could contain a light border that is lit red; white as well as other colors such as blue, green, amber, orange, yellow, etc. can also be used and lit either individually or as combinations of, for example, discrete, blended or mixed, etc. colors or white color temperatures, etc. Note that the power conversion/supply unit can be inside the tubes/bars/etc., external to the tubes/bars/etc., inside the lighting, or combinations of these as well as having some of the parts/components distributed among these, etc. The arrangement(s) and colors discussed herein is/are merely for example purposes and is not intended to be limiting in any way or form.

The lighting for the present invention can be back lit, edge lit, side lit, direct lit, etc. and can be white or any color or combinations of colors or combinations of white and colors, etc. including but not limited to white, red, blue, green, amber, orange, yellow, cyan, etc. The arrangement(s) and choice of colors is/are merely for example purposes and is not intended to be limiting in any way or form.

In another example, the present invention may be used in a hospital or office to minimize the energy cost of occupied and non-occupied space. For example, many hospital rooms have the lights on at all times, as well as climate control and even music to ensure a room is ready for use. Using the present invention the occupancy of the room may be automatically detected resulting in the automatic activation of some or all lighting, climate control, audio, and other devices that contribute to ensuring the room is comfortable. Equipment for example but not limited to such as tablets for reading, TV's, and other devices may also be charged using the renewable energy provided by the solar shades/curtains to further curtail the cost associated with running the room. The system may also control lights in closets, hallways, and other areas that are generally useful only when occupied. The system may also alter the light quality during, for example, quiet hours, to comfortably indicate to the building occupants the time. By using different light colors/wavelengths and intensities many circumstances can be indicated without the need for using an intercom system, or other audible methods of conversation. The present invention can also be used to indicate an emergency by flashing or otherwise displaying a non-continuous or varied light output that is indicative of danger such as, for example, but not limited to, a red or orange or other color or colors. The use of light for indicating time allotments may also be used in, for example, a day-care setting in which the time for naps is indicated by a certain light color, etc. Not only will this simplify the use of scheduling certain events at certain times, but it will in some instances facilitate the event such as, for example, napping. In the same context the system can be used in relaxing environments such as at spas, massage clinics, psychology clinics, headache treatment centers, and many other establishments where light-sensitive conditions can be treated with the use of modulating light colors, intensities, and pulses. In addition, as mentioned elsewhere herein, the color/wavelength(s) range(s) can be used to regulate, control, sync up, reset, etc. circadian rhythms, treat SAD, stimulate or depress melatonin generation, etc. as the situation and circumstances arise within the settings, and locations, and types of environments mentioned and discussed herein including but not limited to hospitals, including, but not limited to neonatal units, intensive care, recovery, surgical, waiting, children, critical care, emergency, urgent care, elderly care, hospice, rooms, etc. as well as cancer treatment, sleep disorder treatment, dementia and Alzheimer treatment and care, libraries, student classrooms and other places of elementary, K12, high school, college and university educational facilities and locations, places of worship, office and business buildings and locations, etc.as well other locations, residences, businesses, temporary housing and shelter, etc.

In the case that smart phones, tablets, computers, or personal devices are not available, the user may use a remote that is created specifically for this system. This remote will allow them to control the system in the same way a smart phone or tablet running a software application would. This remote control will have the ability for the user to customize the buttons and functions of the remote to their preference. It could, for example, have a liquid crystal display (LCD) for readout and stats that will be readable by the user. This LCD screen will also display notifications or alerts for example, if there is a fire, flood, or if motion sensors are triggered, it will notify the user on the LCD screen. The remote could also alert the user with a vibration in the remote control. For example, if the user has the remote in a pocket or on a table the user can feel or hear the alerts or notifications. The remote can also alarm with a sound so the user knows that there is a notification or alert. This remote will connect to the system wirelessly so that it can be used in the building and not have to be in a line of sight to the server or main controller. The remote could also be worn as a necklace, bracelet, watch or on any other appropriate part of the body.

Units employing IR only communications such as space heaters, air conditioners, fans, etc. can be used with remote controls other than smart tablet/cell devices to ensure that the system is usable without additional components. The remote can vary from a simple IR transmitter to a sophisticated wireless transceiver capable of interfacing with the present invention and other automated devices. The remote may include microphone(s) and speaker(s) to employ voice activation/commands in conjunction with controlling a TV, computer monitor, lights, etc. Some embodiments of the present invention do not require a smart-phone or tablet approach to intelligently interface to the control system; other embodiments can use and accept input from both smart wireless devices such as phones, tablets, PDAs, iPod touches, computers, laptops along with devices that are designed to interface with the present invention. Other embodiments of the present invention can interface to other existing wired and wireless sensors, thermostats, heaters, fans, coolers, air conditioners, HVAC, humidifiers, dehumidifiers, television, entertainment systems and components, satellite receivers and remotes, cable box receivers and remotes, smoke and fire detectors and sensors, burglar alarm systems, garage door openers, etc., to be included in and expand upon the present invention including coordinating, scheduling, tasks, synchronizing, sequencing, responding, replying, etc.

The number of IR LEDs in the present invention can be more than one, and may vary as is needed or desired. The IR units may be used to monitor movement and light intensity throughout the system installation and may be used to detect motion/movement for use in triggering specific events such as movement, intrusion and location. This motion information may be used with artificial intelligence of various types and forms to determine what, if any, actions should be taken due to any number of specific events or sequence triggers such as but not limited to alerting the police or other security authorities of an intrusion, opening or closing the garage, turning outside/inside lights on, adjusting interior climate controls, activating a home theater room, etc. In some embodiments of the present invention, if motion is detected either inside or outside or both of the house, residence, business, apartment, condo, room, etc. and that motion is not accompanied by a proper form of identification including, but not limited to, electrical identification, visual identification, optical recognition, pattern recognition, vision recognition, etc., an alarm, warning, including audio warning, strobe lighting, flashing lights, color changing lights, lights being turned off, etc. may occur or select individuals including neighbors, family and friends and others may be notified by e-mail, web content, web alert, phone calls, text messages, video transmissions, etc. In some embodiments of the present invention, if smoke or fire is detected, the lights that are connected to the present invention may flash on and off, may change color, may dim and then go brighter, etc. as well as speakers issuing warnings and contacting friends and family, neighbors and others as well as, in some cases, 911 or the fire department, combinations of these, etc. Radio frequency identification (RFID), Bluetooth or all types and forms including Bluetooth Low Energy (BLE) and similar such systems can be used with the present invention to turn on or off or dim embodiments and implementations of the present invention remotely.

In some embodiments of the present invention, motion throughout a house or other area can be predicted or otherwise analyzed based on the input from multiple sensors such as, but not limited to, multiple motion detectors. For example, if signals are detected from multiple motion detectors, the sequence and timing of the signals from the motion detectors can be analyzed to determine not only movement but also path, speed etc. of a person, animal etc. Such information can be used for a number of applications, such as, but not limited to, predictive actuation of lighting or other devices, for example to turn on lights along the predicted path based on previously detected signals, or to distinguish false alarms from actual detected motion. When combined with identification devices such as RFID, mobile phone signals, or other identifiers carried by users authorized to be in the area, such path predictive analysis can also be used to determine unauthorized persons moving toward restricted or private areas, possibly triggering audible warnings against proceeding or other actions such as locking of doors, flashing of lights, triggering of alarm systems, etc., to prevent unwanted intrusion.

The automation system disclosed herein can be adapted to interface and interact with other systems, such as, but not limited to, other home automation systems, temperature control systems, lighting control systems, communication systems, entertainment systems, security systems, fire and protection systems, cable and satellite systems, etc.

This invention can be equipped with light-emitting diodes (LED), organic light-emitting diodes (OLED), QDs, or other types of lighting and/or LEDs. These LEDs can be dimmed, brightened, turned on/off, rotated, turned, moved, change color, etc., wirelessly through a network that is comprised of wireless signals and command data that is transmitted and received via a server or main control unit which can be connected to a wireless local area network (WLAN), where it can be utilized and controlled by smart phones, tablets, personal device, computers, etc. These commands and communications can be controlled and managed through software applications designed on platforms like Android, iOS, C++, and Java using a graphical user interface (GUI). This main control unit can be used to communicate data and commands to and from the lamps, lights, light fixtures, ballasts, desk lamps, etc. This system can also be able to communicate commands to these devices and can be remotely controlled from anywhere where there is a signal from the WLAN, for example, they can be dimmed, turned on and off, the color can be changed, etc. The main control unit or server can have capabilities of handling Internet Protocol (IP), Transmission Control Protocol (TCP), and User Datagram Protocol (UDP) network information from the user's smart phone, tablet, personal device, computer, etc. This main control unit or server can then output commands or data through a wireless protocol to the remote units which can then respond to the commands sent by carrying out tasks such as switching a device on/off or other various tasks. Another way of controlling the remote devices in a wireless manner is using the Bluetooth protocol. This allows the user to connect to the lamps, lights, light fixtures, ballasts, desk lamps, etc. directly from the smart phone, tablet, personal device, computer, etc., without having to connect to the main control unit or server. It is also possible to connect the server to the remote devices via the power lines that already exist in the home or business.

These lamps, lights, light fixtures, ballasts, desk lamps, etc., can interact with a user without using a main control unit or server. For example, the remote device can sense when a user enters a room by using Bluetooth by recognize the user's Bluetooth unique universal identifier (UUID) and adjust the lamps, lights, light fixtures, ballasts, desk lamps, etc., to the user's liking based on personal settings they set-up on their device, which contains a Bluetooth radio. For example, the lamps, lights, light fixtures, ballasts, desk lamps, etc., can dim to the preset dimming level set on a smart phone, tablet, personal device, etc. A desk lamp, for example, can move, due to the internal servos on the arms and change the brightness of the light to a desired position when a user enters a room or building. The Bluetooth can automatically connect to the lamp, light, light fixture, ballast, desk lamp, etc., and then the application on the personal device transmits the desired preset data to the lamp, light, light fixture, ballast, desk lamp, etc. This system is not limited to lamps, lights, light fixtures, ballasts, desk lamps, etc. The smart phone, tablet, personal device, etc., may connect to many lights at once and send the same command to all or it can send commands individually to many different lights. For example, if a user walked into a large warehouse or business, specific lights can be turned on automatically or manually by the user. The user can set-up the application to turn on lights only in the rooms the user is occupying, therefore, conserving energy by turning off the lights when the user leaves a room and turning on the lights in the room that the user is entering automatically by way of the software application that connects to the lights wirelessly. Another example, the lights can be programmed to turn on when a user enters their driveway or garage while in their vehicle. This way they can see where they are parking etc.

This invention can be equipped with motion sensors to detect motion in rooms, home/business exteriors, hospitals, schools, etc., to turn on lights or brighten lights for individuals passing by. These motion sensors can also aid in the exposure of intruders lurking outside and inside of buildings, homes, businesses, etc. Motion sensors can be used for security purposes on the exterior of a building for example. When motion is detected the blinds/shades can close as to not allow individuals to have a visible line of sight into the home, office, or building/structure through the windows. Lights can be triggered to turn on in a building when motion is detected outside to imitate the idea that the building is being occupied, for example. These motion detectors can also sense motion made by vehicles and animals.

The servos in these lights can change the lighting scheme in a room or building by physically relocating the light source and/or where the light is aimed, focused, directed, etc. For example, a desk lamp can be controlled to move the LED and/or 180 degrees to point up at the ceiling or down at the desk or below. The lamp position can be changed wirelessly on a smart phone, tablet, personal device, computer etc., for example using a software application installed on the device.

These light movements can also be preprogrammed by the user to move, dim, turn on/off, etc, at scheduled times, days, months, and years, allowing the user to set the lights to turn on in the morning or flash repeatedly or, in other scenarios, slowly increase in light intensity to wake the user up, for example. The lights can also be set to change color/wavelength as part of waking up in the morning and preparing to and/or falling asleep at night or in the evening. The lights can be set up to turn on while the user is away from the home or business as to give the impression the building is occupied for security purposes, for example. The light can be scheduled to change direction at specific times during the day or week. For example, the lights can turn up the power and lumen intensity aimed for example at the ceiling in the evening to change the ambiance of the room. As another example, the lights could soften, dim and/or change color(s), etc. in the evening to provide different ambiances and mood shifts as well as health care benefits. The light(s) can be scheduled to turn on specific colors at specific times during the day, week, month, or year. For example, the light can turn on a blue, red, green, yellow, pink, orange, etc., LEDs, OLEDs, QDs, other SSLs, other lighting, etc. in the evening or day time or night time. The lights can be programmed to change through the different colors smoothly or the colors can be changed randomly automatically to the users' wants desires or to support healthy living and lighting.

The present invention can include voice recognition and can respond to vocal commands given to the light, temperature, humidity, environment, ambiance, etc. combinations of these in any sequence or schedule or dependency, etc. For example, the light can be dimmed, brightened, turned on/off, rotated, turned, moved, change color, etc., by voice commands from the user. The light can be across a room or on a desk, on a wall, in a fixture and respond to voice commands. These lights can vary from and include, but are not limited to, lamps, lights, light fixtures, ballasts, desk lamps, etc.

These lamps, lights, light fixtures, ballasts, desk lamps, etc., can transmit information back to the user. Such information includes but is not limited to battery voltage, current usage, power, state, etc. They also have the ability to transmit and receive data in order to carry out their programmed functions. This data can be presented on the user's device through a GUI and managed by the software application running on the user's device. These remote devices allow the user to control a multitude of devices, as mentioned previously, in their home and/or business, etc. The software applications also allow the user the ability to program automated functions into their remote devices. For example, the system can control and automate the lighting when the user is away from the home or business. Having the device connected to the Internet through an Internet router, the user can be able to control their remote devices on the wide area network (WAN) from outside their home or business network when the user is away.

This invention is not limited to one LED, OLED, etc. It can have various lights in/on one lamp, light, light fixture, ballast, desk lamp, etc. The user can have the ability to control the various lights on the fixture via a smart phone, tablet, personal device, etc. These auxiliary or alternative LEDs, OLED, QDs, can be in various places on the lights, fixtures, body, etc. They can be controlled independently from each other or altogether or in a diverse number of sequenced and scheduled events, etc. As an example, they all can be set according to a scheduled time to flash, turn on/off, dim, brighten, etc. These lights can be voice controlled as well.

In conjunction with solar charging blinds in the window, lights of the present invention can be connected to the battery that is being charged by the solar panels and become illuminated at night. The user can close these blinds remotely with the use of a smart phone, tablet, personal device, computer etc. The blinds/shades/drapes/curtains/shutters/etc. can be programmed to close and turn on the lights at the same time in the evening for example. The blinds/shades can be powered from a wall socket or by battery so they can be placed in hard to reach places without the need for a wall outlet. The blinds/shades are connected to the same network as the lights so they can all work together with each other and can, for example, all be controlled by the same device, by the user, etc. The blinds or shades can be controlled by a server or Bluetooth similarly to the lighting.

This invention is part of a connected network therefore, when the solar powered battery is running low on power, it can communicate to the light that is being powered from the battery to dim if the light is on, in order to conserve power from the battery. Similarly, if the batter is running low on power and multiple devices are plugged into the battery, the light can dim to a lower level or send an alert notification to the user that the battery is getting too low to power some or all devices and that an alternative power source may be needed. The battery can also be set up to have priorities as far what to power from its source. The battery can cut off power to devices in a set priority or restrict current to those devices in order to provide power to the devices with a higher priority.

The lighting devices can also have solar panels on them to collect power and charge an internal battery. This benefits light fixtures that are for example, on the interior and on the exterior of a building in a well sunlit area. In some cases, the light may not need to be connected to a main power source.

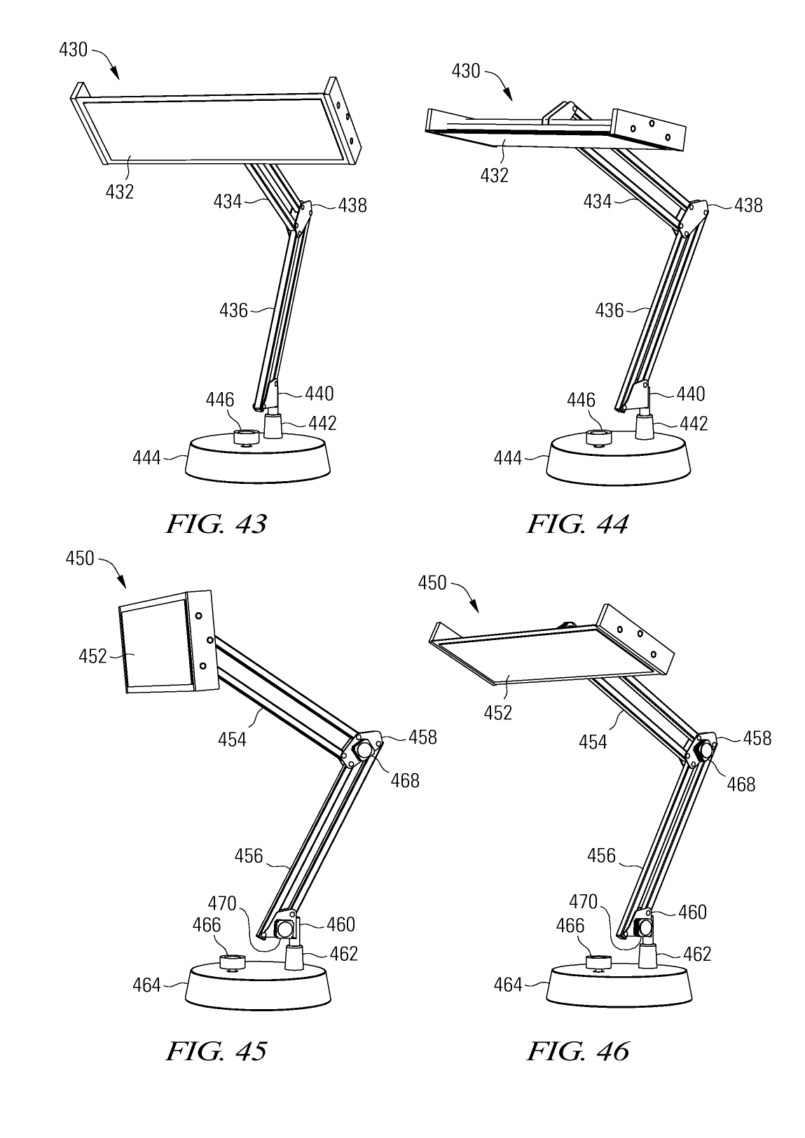

Turning to FIGS. 43-44, an articulating desk lamp 430 with one or more rotating solid state lighting panels 432 is depicted in accordance with some embodiments of the invention. The desk lamp 430 can be mounted on one or more support members 434, 436 connected by hinges 438, 440 and mounted by a rotating sleeve 442 to a base 444, allowing the lighting panel 432 to be pointed in any desired direction. The support structure is not limited to the articulating arm assembly shown in FIGS. 43-44, but can include any device or assembly suitable for positioning and orienting the lighting panel 432, such as, but not limited to, a ball and socket chain, gimbaled arm, etc. A power supply/dimming control circuit can be provided to power and control the lighting panel 432 and can be positioned in any suitable location, such as the base 444. An IR receiver (not shown) and/or other wired or wireless connection can be provided to link the desk lamp 430 to other parts of an automation system, enabling the illumination level, color, on/off state to be controlled, scheduled, sequenced, etc. The position and/or orientation of the lighting panel 432 can be automatically controlled in some embodiments by motors (e.g., 468, 470) such as stepper motors, DC motors or other actuators as shown in FIGS. 45-46. For example, IR receivers are provided on the motors (e.g., 468, 470) and/or motor controllers in some embodiments to remotely control/schedule motor movements. Encoders, decoders, etc. can be used to monitor, track, store, record, remember, replay, move to, etc. existing and previous positions, locations, etc. and can also be used to respond to, interact with, track, move, position, etc. the present invention depicted in FIGS. 43-46 based on, for example, but not limited to one or more inputs, information, sensing, detection, time of day, date, ambient temperature, light intensity, movement, proximity, location, GPS information, atomic clock information, etc.

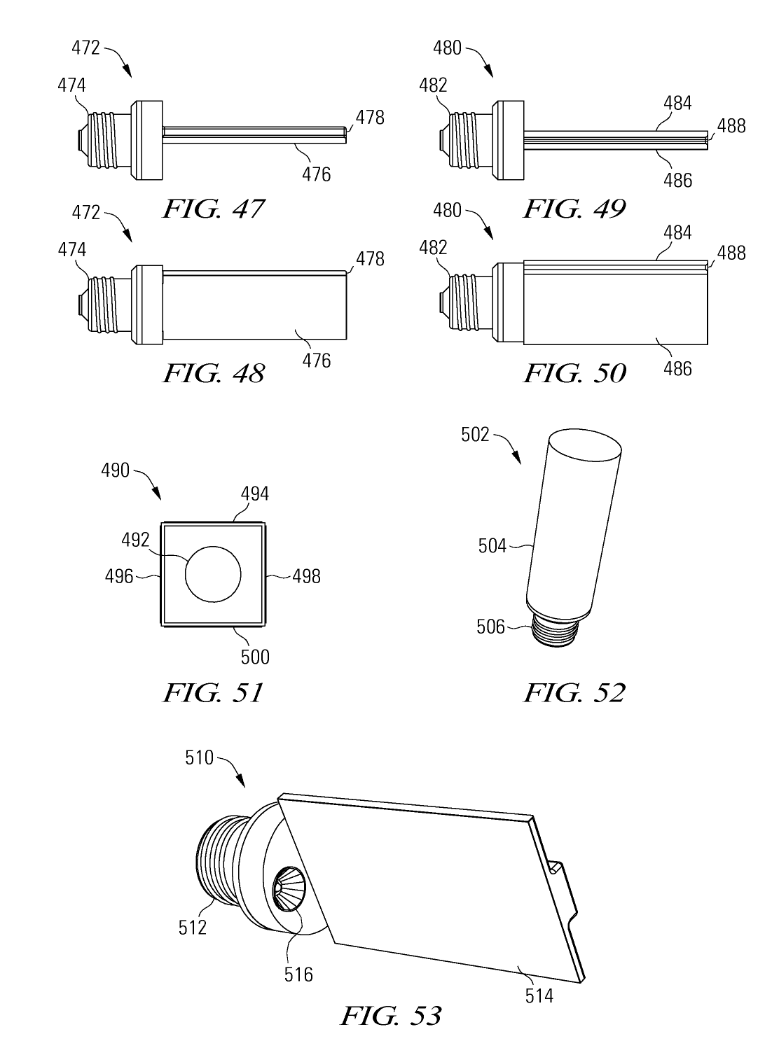

Turning to FIGS. 47-53, solid state lighting can be combined with standard lamp bases for use in existing light fixtures. For example, a single OLED panel 476 can be mounted to an OLED substrate 478 on a lamp base 474 as in the light 472 shown in FIGS. 47-48. The number of lighting panels included can vary from one to any number, such as the two back-to-back OLED panels 484, 486 mounted to OLED substrate 488 on base 482 as in the light 480 shown in FIGS. 49-50, or four OLED panels 494, 496, 498, 500 mounted to base 492 in the light 490 of FIG. 51.

In some embodiments, a SSL, a LED, a QD and/or an OLED or combinations of these, etc. A-lamp can swivel about the axis of the socket. Embodiments of the present invention may also use motors, actuators, etc. to tilt, move, angle, etc. the OLED (or LED or both or other SSL including QD) lighting. The internal power supply is contained within the socket and, optionally, other portions of the light. The internal drivers are dimmable, high efficiency and high PF. Protective covers can be provided, such as, but not limited to, the cylindrical cover 504 mounted to the base 506 in light 502.

In some embodiments, as in FIG. 53, a white LED (e.g., 516) and an amber OLED (e.g., 514) are used to provide white light ‘daylight’ and amber light ‘nightlight’ to support, for example, circadian rhythms and other health effects at work places, homes, hospitals, etc. In other embodiments of the present invention, the OLEDs may be replaced or augmented with either white LEDs (or any other color) or amber or RGB and/or RGBA LEDs (or other SSL devices including but not limited to QDs) to perform the T8, T12, T5, U shaped or other fluorescent lamp replacement, etc. Other embodiments of the present invention may employ wireless power transfer such as inductive coupling or resonant coupling to remotely power the OLEDs or LEDs.

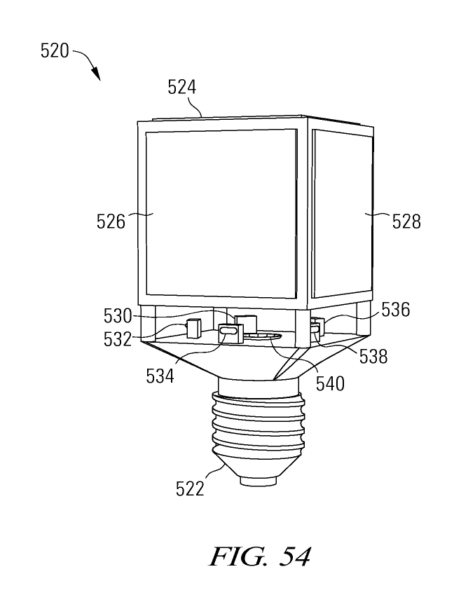

Turning to FIG. 54, a light 520 with one or multiple solid state lighting panels (e.g., 524, 526, 528), a point light source (in reflector cup 540) such as an LED or LEDs and IR receivers 530, 532, 534, 536, 538) is depicted in accordance with some embodiments of the invention. The light 520 can be turned off, dimmed, and the color can be controlled in some embodiments based on commands received by IR receivers 530, 532, 534, 536, 538). A power supply/dimming/monitor control circuit can be located in the lamp base 522 and/or behind the solid state lighting panels (e.g., 524, 526, 528), along with a heat sink as needed or desired. The light 520 can include any number of solid state lighting panels (e.g., 524, 526, 528), such as, but not limited to, 5 forming an open-base cube as in FIG. 54, or any other number and configuration of SSL panels. Similarly, any number, type and positioning of point light sources can be included, such as white LEDs (or any other color) or amber or RGB and/or RGBA LEDs (or other SSL devices including but not limited to QDs). In some embodiments of the present invention, only one or more than one but less than 5 SSL panels such as OLED panels are used to provide light. In some embodiments this light may be a different or same color temperature white light; in other embodiments this SSL panel such as OLED panel lighting may be, for example, amber or close to amber (i.e., orange or yellow) OLED lights such that the light can be a rather high power/high intensity ‘white’ light when needed and then switch to an amber light that can support and enhance circadian rhythm cycles and alignment, etc. as well as good sleep to aid in mitigating, eliminating or reducing sleeping disorders and other illnesses. In some embodiments of this present invention, one or more amber panels can be used with, for example, a white LED such that the amber panels when not powered are mirror-like and reflective such that the light from the example LED to reflect the LED light out where there are no OLED panels but only transparent material so as to effectively increase the useful lumens of white light including dimmed white light that are delivered outside of the present invention. Both the white light (i.e., LED) and, in this example, the amber panel light (OLED or LED edge lit, edge emitters, front emitters, back emitters, etc., combinations of these) can be dimmed including remotely dimmed including but not limited to dimmed and/or turned off based on time of day, information including but not limited to sensor and detector of any and all type(s) information, web-based information, weather, etc., user input and programming, others' input including medical staff, caregivers, family and friends, hospital staff, etc. input, directions, control, monitoring, and programming, etc., combinations of these, etc., including remotely, locally, etc. In other embodiments of the present invention, the SSL panels can be replaced with SSL or other point sources including LEDs and QDs, etc. which can perform the similar function and operation as the SSL point and panel sources including having white and amber, white and RGB and amber, other colors, other combinations of white, amber, RGB point sources and panels, etc. An example of a potentially beneficial circadian rhythm and/or sleep aid would be embodiments and implementations of the present invention that, for example produce white or blue, combinations of these, etc. wavelength light in the morning to wake up with and produce amber, yellow, orange, red wavelength light, combinations of these, etc. at night. In the case of a night shifted person or worker, the opposite may be apply in that amber or similar wavelength lighting would be used when the person returns in the late night or early morning to promote proper circadian rhythm and sleep patterns without interfering with melatonin cycle production and then wake up to bright white and/or blue light or be woken up with a gently increasing white or blue light that, in some embodiments could be set, programmed, sequenced, etc. to start out with amber or near-amber wavelengths and then progress to white/blue light, etc. The present invention can also be designed to turn on either fully or to a dimmed level the amber (and similar) wavelength lighting should a person awake at night and, for example need to get out of bed or find something and then go back to bed and sleep. Such turned on or dimmed on amber, yellow, orange, red, etc. wavelength lighting could be motion sensed, detected and activated, REM or other sleep pattern sensed, detected and activated, sound sensed, detected and activated, voice sensed, detected and activated, voice recognition sensed, detected and activated, gesture sensed, detected and activated, remote set and activated, time of night (or day) sensed, detected and activated, medically detected or sensed and activated, wearable electronics, etc. sensed, detected and activated, brain activity sensed, detected and activated, etc. and combinations of these, etc. In some embodiments of the present invention the light can consist of SSL panels, for example, OLED panels, that have, for example, both blue and amber OLEDs that are separately addressable and can be turned off or dimmed individually to provide the same blue/white and amber lighting discussed above for supporting circadian rhythm and sleep health, wellness and well-being. Although FIGS. 53 and 54 show essentially rectangular and square lights, respectively, the present invention can be virtually any shape including the more common and practical ones like an A-lamp, PAR 30, PAR 38, R30, can lamps, down lights, accent lights, under cabinet lights, accent lights, sconces, pendants, chandeliers, track lighting, hallway lighting, patio lighting, entrance lighting, M16, etc. round, square, cylindrical, rectangular, etc. as well as U-shaped, linear such as T8, T5, T12, linear however more two dimensional like a panel, etc., combinations of these, etc. The SSL lights can also be adapted for and installed in ceiling lights or other fixtures, whether recessed or mounted to a ceiling or wall, including but not limited to ceiling fixtures with decorative and/or functional covers or diffusers. SSL lights can be installed in any orientation in fixtures, such as horizontally or vertically or any other orientation.

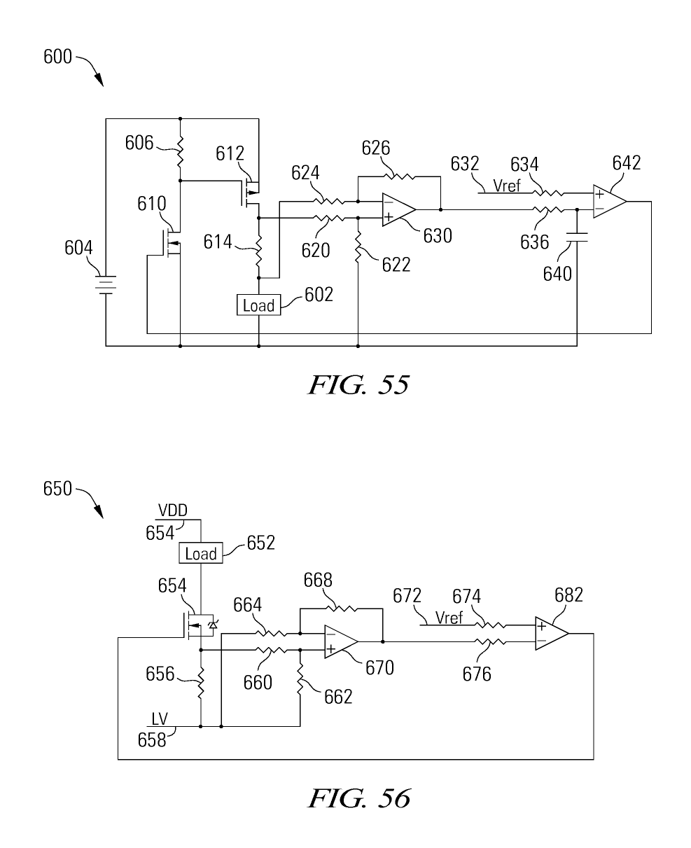

FIGS. 55-58 are circuit diagrams of power supply/dimming control circuits for solid state lighting devices such as LED, OLED, QD, etc. in accordance with some embodiments of the invention. Turning now to FIG. 55, a power supply/dimming control circuit 600 is illustrated in accordance with some embodiments of the invention. A load 602 such as, but not limited to, an LED or OLED or QD or other solid state light or combinations of these, etc. is powered from a DC source 604 that can be generated or provided in any manner including from an AC source such as wall line power, an inverter, etc. or a DC source such as a solar cell or cells or batteries, fuel cells, DC to DC converter, etc. An electrical current to the load 602 flows through a transistor 612 and a low value current sensing resistor 614. The transistor 612 is a P channel metal oxide semiconductor field effect transistor (MOSFET), but is replaced with a PNP bipolar junction transistor in some embodiments. The transistor 612 is controlled by a feedback signal from an error amplifier 642, via transistor 610 and resistor 606. A difference amplifier 630 amplifies the voltage across current sense resistor 614, with the gain controlled by resistors 620, 622, 624, 626. This voltage is optionally filtered by resistor 636 and capacitor 640, and is compared by the error amplifier 642 with a reference voltage 632 provided through resistor 634. In some embodiments, the power supply/dimming control circuit 600 operates as a linear regulator, but can also be adapted as a switching regulator, PWM controlled circuit, etc.

Turning now to FIG. 56, another power supply/dimming control circuit 650 is illustrated in accordance with some embodiments of the invention. A load 652 such as, but not limited to, an LED or OLED or other solid state light is powered from a DC source 654 that can be generated or provided in any manner. Current through the load 652 is controlled by transistor 654 based on an error feedback signal from difference amplifier 682. Current through the load 652 is measured by current sense resistor 656, amplified by difference amplifier 670, with the gain set by resistors 660, 662, 664, 668. The resulting signal is compared by the error amplifier 682 with a reference voltage 672 provided through resistor 674. Node 658 provides a low voltage reference, such as, but not limited to, a ground. In some embodiments, the power supply/dimming control circuit 650 operates as a linear regulator, but can also be adapted as a switching regulator, PWM controlled circuit, etc.

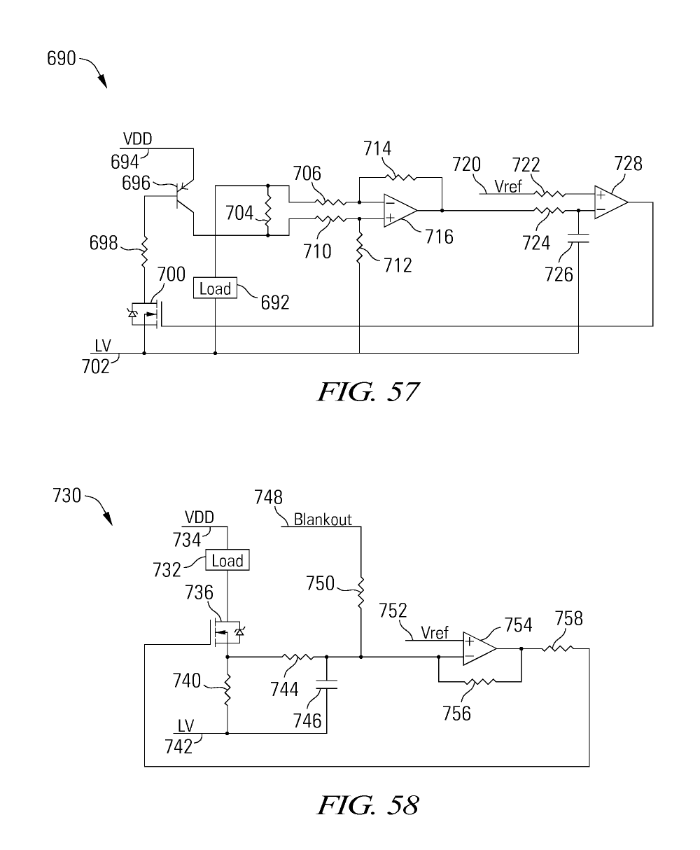

Turning now to FIG. 57, another power supply/dimming control circuit 690 is illustrated in accordance with some embodiments of the invention. A load 692 such as, but not limited to, an LED or OLED or other solid state light is powered from a DC source 694 that can be generated or provided in any manner. Current through the load 692 is controlled by BJT transistor 696 based on an error feedback signal from difference amplifier 728, via transistor 700 and resistor 698. Current through the load 692 is measured by current sense resistor 704, amplified by difference amplifier 716, with the gain set by resistors 706, 710, 712, 714. The resulting signal is optionally filtered by resistor 724 and capacitor 726 and compared by the error amplifier 728 with a reference voltage 720 provided through resistor 722. Node 702 provides a low voltage reference, such as, but not limited to, a ground. In some embodiments, the power supply/dimming control circuit 690 operates as a linear regulator, but can also be adapted as a switching regulator, PWM controlled circuit, etc.

Turning now to FIG. 58, another power supply/dimming control circuit 730 is illustrated in accordance with some embodiments of the invention. A load 732 such as, but not limited to, an LED or OLED or other solid state light is powered from a DC source 734 that can be generated or provided in any manner. Current through the load 732 is controlled by transistor 736 based on an error feedback signal from summing error amplifier 754, via resistor 758. Current through the load 732 is measured by current sense resistor 740, with the current measurement optionally by capacitor 746 along with input resistor 744. Error amplifier 754 compares the current measurement voltage with a voltage reference 752, with the gain set by optional feedback resistor 756. A blankout signal 748 can be applied through resistor 750 to an input of the error amplifier 754 to turn off or limit the current through the load 732. Node 742 provides a low voltage reference, such as, but not limited to, a ground. In some embodiments, the power supply/dimming control circuit 730 operates as a linear regulator, but can also be adapted as a switching regulator, PWM controlled circuit, etc.

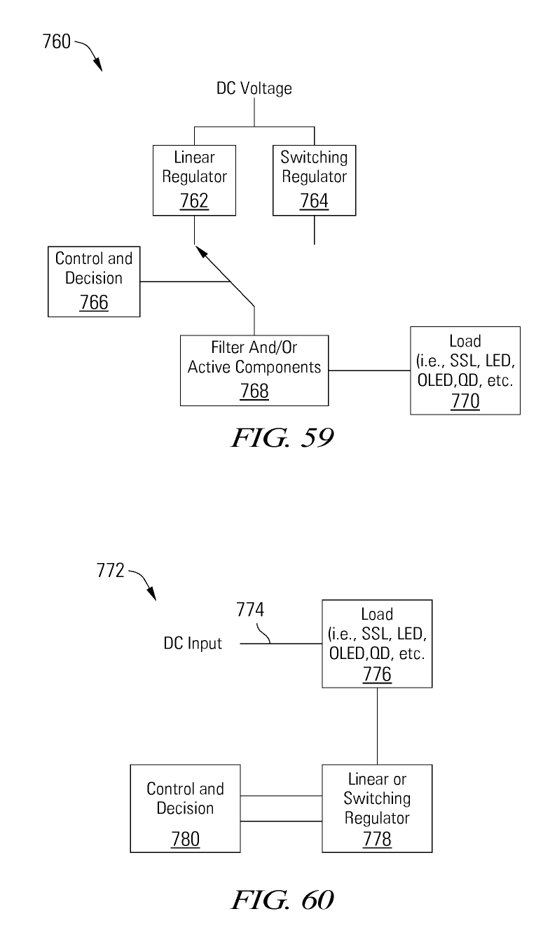

Turning to FIG. 59, a block diagram of a power supply/dimming control circuit 760 with selectable linear and switching regulation for solid state lighting devices such as LED, OLED, QD, etc. is depicted in accordance with some embodiments of the invention. A DC power input can be regulated by either a linear regulator 762 or switching regulator 764, as selected by a control and decision circuit 766. The resulting power signal can be filtered or otherwise processed by circuit 768 and provided to load 770. The selection between linear and switching regulation can be made dynamically, for example based on the dimming level or any other criteria. For example, the switching regulator 764 can be selected when performing digital deep dimming to prevent color changes in an OLED, LED or other SSL being powered by the system at low to very low current/power levels or, for example, analog dimming at higher power levels or, in the situation, where analog dimming is preferred/desired for whatever reasons.

Turning to FIG. 60, a block diagram of another power supply/dimming control circuit 772 for solid state lighting devices such as LED, OLED, QD, etc. is depicted in accordance with some embodiments of the invention. A load 776 is powered from a DC input 774 or other suitable power input for which such input shown in the block diagram FIG. 60 originating for example, but not limited to an AC wall line power source, an AC inverter, a DC source such as a battery or batteries, fuel cells, solar panels, DC to DC converters, etc., controlled by a linear or switching regulator 778 based on a control and decision circuit 780.

Excessive use of electricity in consumer residential housing and commercial and industrial buildings in the United States has negative effects including the excessive and unnecessary use on the United States economy. Energy consumption by buildings account for close to 40% of the total energy consumed and use almost 70% of all the electricity used in the United States. They are also responsible for nearly 40% of carbon dioxide emissions and close to 50% of sulfur dioxide emissions. It is therefore necessary to reduce the energy consumed by residences and buildings. A major source of this excessive and wasted electrical energy is inefficient lighting. This source of waste creates an opportunity for highly efficient and innovative replacement solutions.

Current lighting technology has limitations that leave many lighting customers' needs unmet. However, most customers in the High CRI commercial lighting market find the best available alternative to be T8/T5/TSHO fluorescent luminaires. A reduced total-cost-of-ownership approach made possible by the

the use of SSL combined with power supplies and drivers will permit customers and end-users, who are also concerned with the energy and maintenance costs of their lighting installations, to reap substantial benefits compared to other legacy and emerging light source technologies and luminaires. Performance benefits include potentially increased energy savings over many other light source technologies including with OLEDs as OLEDs do not require diffusors, higher levels of sustainability, and improved lighting quality. In addition, user-adjustable, -friendly and powerful control and monitoring lighting options enabled for the SSLs including but not limited to LEDs, OLEDs and QDs by the present invention will further enhance the user experience while reducing energy consumption especially when dimming.

White OLED, white-changing/tunable OLED and/or RGB OLED and/or one or more colored LEDs as well as other SSL, etc. in the present invention provide for tunable light bulbs, task lights, desk and task lamps, table lights and lamps, down lights and luminaires as well as for outdoor lighting including street lamp and parking lot lighting.

Implementations of the present invention include intelligent white and color changing SSL including but not limited to LED, OLED, QD, other SSL, combinations of these, etc. for desk and task lamps, table lamps, floor lamps, wall lamps and lighting, ceiling lamps and lighting, floor lamps and lighting, sconces, pendant, etc. lighting that also can be directly and relatively-easily applied to other products including under-cabinet and over-cabinet lighting for kitchens and bathrooms, vanity application, accent lighting, etc. An example of the present invention includes a desk lamp that is locally and remotely dimmable. Another example is a 12 channel common cathode OLED driver in which each channel/all channels can be separately addressed, controlled and dimmed. All of the above can be wirelessly interfaced, controlled and monitored using, for example, smart phones (i.e., iPhones, Droids), tablets (i.e., iPad, iPod touch, droid, Kindle, Samsung, etc. tablets), laptops, desktops and other such digital assistants. The universal drivers can also support Triac and 0 to 10 Volt dimming as well as optional powerline (PLC) and wired and/or wireless remote control.

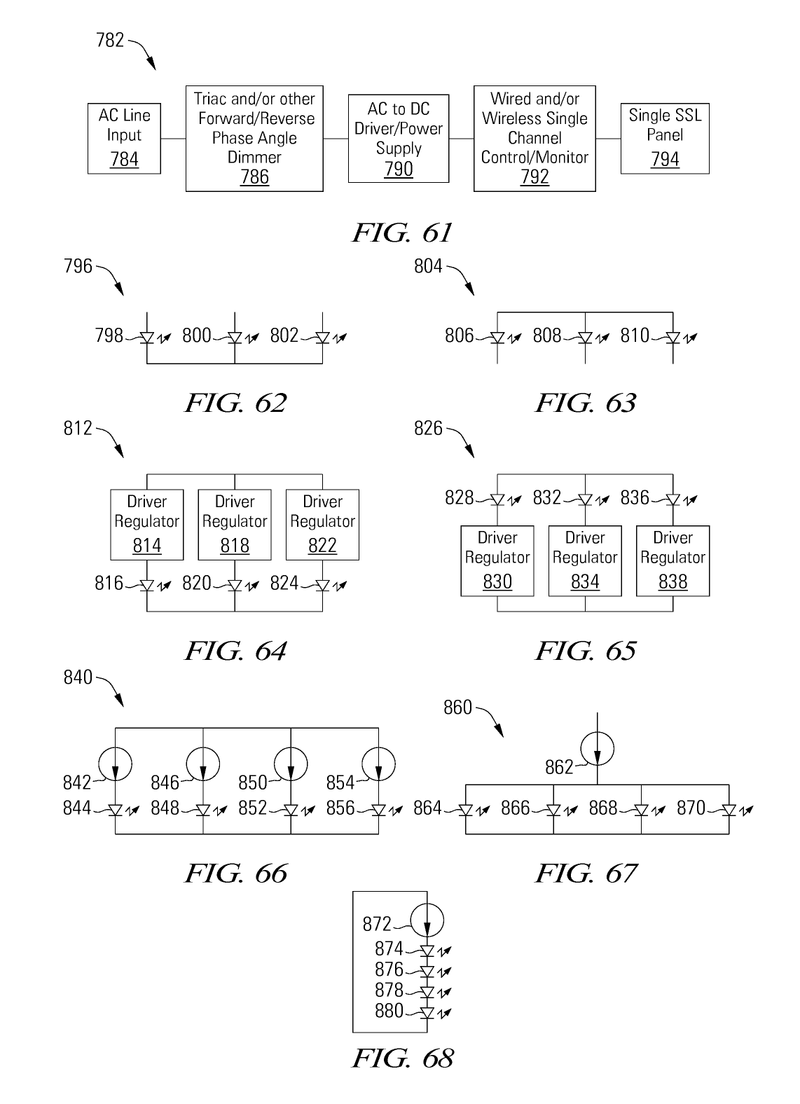

Turning now to FIG. 61, a block diagram of a solid state lighting power supply/dimming circuit 782 is depicted in accordance with some embodiments of the invention. The power supply/dimming circuit 782 powers a single monochrome (i.e., white) OLED (or LED or QD) panel 794 from an AC line input 784 via an AC to DC driver/power supply 790 that can be dimmed using a Triac dimmer and/or other types of forward/reverse phase angle/cut dimmer 786 and also controlled/dimmed and monitored by wired and/or wireless (or both) interfaces 792.

An OLED panel can consist of, for example, more than one stack, stripe and/or color that is separately electrically contacted and isolated from the others. Such a situation can arise, for example, when more than one OLED color (or set of electrodes) is being driven by a power supply. Typically in such situations and arrangements the electrodes are configured so that there is a common electrode (i.e., a common cathode or a common anode configuration) such that all stripes, colors, etc. share a common electrode which could be either the anode or the cathode of the OLED panel. An example illustration 796 of a common cathode configuration for three OLEDs (or LEDs) 798, 800, 802 having common cathode connections is shown in FIG. 62. An example illustration 804 of a common anode configuration for three OLEDs (or LEDs) 806, 808, 810 having common anode connections is shown in FIG. 63. Example control topologies 812, 826 for each are shown in FIG. 64, in which driver regulators 814, 818, 822 are connected in series with each of the common cathode OLEDs (or LEDs) 816, 820, 824, and in FIG. 65, in which driver regulators 830, 834, 838 are connected in series with each of the common anode OLEDs (or LEDs) 828, 832, 836.

With LEDs, typically both the cathode and anode for each individual LED color are available to put in parallel and/or put in series either individually or in groups/arrays/etc. multiple LEDs such that often there are only two electrical power connections from the power to the LEDs and therefore the power supply/driver output and output connection configurations are often much simpler and more universal for LEDs than OLEDs. With the continued widespread growth and use of LEDs, there are and will be numerous exceptions to just the two connections per LED fixture or luminaire, although such a generalization usually applies.

Any practical number of channels from 2 to hundreds or thousands of channels with, for example, 12, 16, 32, 64, 100, 128, 250, 256, 500, 512, and so on can be implemented.

Embodiments of the present invention include smart remote dimming of, for example, an SSL including but not limited to LED, OLED, QD, combinations of these, etc. for desk/task lamps and all other types of lamps, lighting, luminaires, etc. discussed herein that is locally and remotely dimmable with power supply/driver design considerations that include, but are not limited to, high PF, low THD, low EMI, dimming, flicker-free operation and high to extremely high efficiency.

The present invention includes multi-panel configurations including parallel (i.e., same voltage, shared total current through each panel) and series (i.e., same current, stacked voltage). In FIG. 66, a circuit 840 includes multiple SSLs 844, 848, 852, 856 in parallel with a same voltage, each with an individual current control 842, 846, 850, 854. In FIG. 67, a circuit 860 includes multiple SSLs 864, 866, 868, 870 are connected in parallel with a same voltage and a shared current control 862. In FIG. 68, multiple SSLs 874, 876, 878, 880 are connected in series with a same current 872 and stacked, different voltage. Typically most OLED panels, whether single or multi-color, operate at a total voltage of less than 20 VDC and are typically connected in parallel as shown in FIG. 68. FIG. 68 is typical of a two terminal/electrode LED power supply with anode (+) and cathode (−) connections.

Various embodiments of the present invention can have OLED panel configurations for both parallel (i.e., common voltage) and series (i.e., common current) fixed white, white changing, amber and RGB, RGBA, WRGBA, etc. LED, OLED, QD, other SSL, etc. combinations of these, etc. lighting and light panels, lamps, combinations of these, etc.

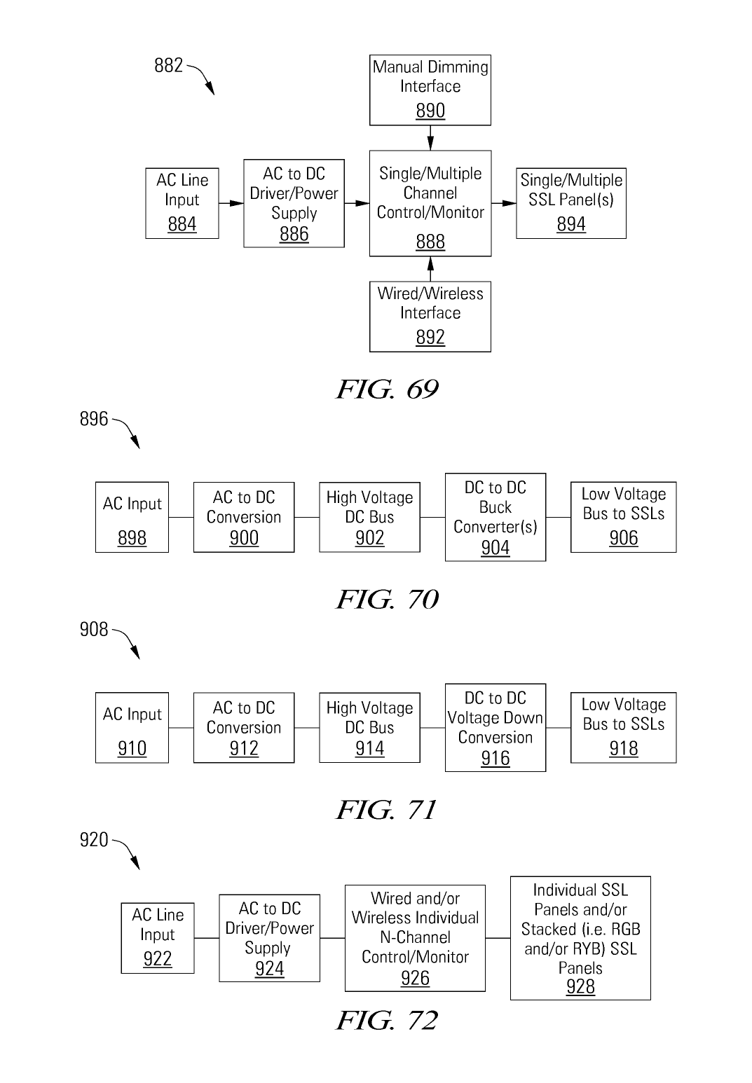

The remote control and monitoring wired/wireless interfaces can also have manual/local control and dimming via, for example, a potentiometer or encoder/decoder. As shown in FIG. 69, a power supply/dimming control circuit 882 for a solid state lighting device 894 can support both manual/local dimming 890 which can be selectively allowed or overridden by a wired and/or wireless interface 892 in accordance with some embodiments of the invention. Power from an AC line input 884 can be converted in an AC to DC driver/power supply 886, with current control and monitoring 888 provided for each channel of an SSL lighting device 894.

For some of the embodiments of the present invention, a simple interface can be used as an OLED identification system that allows the power supply/driver and each of the individual OLED panels to communicate with each other to identify the current and voltage requirements of the respective OLED panels. This allows multiple OLED panels to be connected to the OLED power supplies and drivers safely and correctly. This simple interface can use an OLED identification system that allows the power supply/driver and each of the individual OLED panels to communicate with each other in, for example, but not limited to, a similar but much simpler (and slower) fashion as, for example TIA/EIA485 also known as RS485 interface 2 wire systems using a very simple two wire protocol that is also inexpensive and small size and does not permit power to be supplied to the LED, OLED, QD and/or other SSL, etc. combinations of these, etc. panels as well as being compatible with other standards such as DMX, DALI, 0 to 10 V, other standards, protocols, interfaces, etc. discussed herein.

Some embodiments and implementations of the present invention include smart SSL including but not limited to LED, OLED, QD, other SSL, etc. combinations of these, etc. driver monitoring and logging of pertinent data and parameters including input current, input voltage, inrush current, voltage spikes, power factor, true input power, Volt-Amp (VA) input power, PF, output current, output voltage, output power, etc. Most of these parameters and especially the input parameters can be transmitted either as waveforms (e.g., amplitude vs. time) or as instantaneous or average data points.

Implementations of the present invention can also address LED, OLED, QD and other SSL aging effects on power supply voltage compliance, intelligently addressing for example but not limited to OLED lifetime issues including, but not limited to, color degradation, lifetime lumen depreciation, OLED voltage increase over lifetime, blue OLED issues, etc.

In some embodiments of the present invention wired/wireless interfaces can be designed to accept a ‘daughter card or cards’ that contain the desired wired and/or wireless interfaces which could include, but are not limited to, analog and/or digital interfaces such as 0 to 10 V, DALI, DMX, RS485, etc., with wireless including industrial, scientific and medical (ISM) radio bands/frequencies, Bluetooth, IEEE 802, WiFi, ZigBee, ZWave, etc. The LED and OLED power supplies/drivers/modules can also be Triac/Forward/Reverse phase angle or powerline control (PLC) dimmable (Note: phase angle/cut when dimming result in poor PF and high THD thus defeating the high PF requirement, whereas the other methods of dimming maintain high PF/low THD when dimmed) including forward/reverse phase angle dimming that are compatible with each other and can use the same wireless/wired platforms and interfaces—all of which can be controlled/dimmed/monitored by smart phones, tablets, PDAs, laptops computers, servers, custom remote controls, etc.

Initial configurations of addressing issues involving multiple to numerous OLED panels that collectively constitute a luminaire especially issues directly involved with power distribution to the OLED panels. As mentioned previously, OLEDs to date tend to be low voltage and often paralleled when more than one panel is used in a lighting/luminaire application.

Turning to FIGS. 70-71, higher power applications where, for example, 10 to 20 (or more) panels are used in a single luminaire, can be supported by making the overall AC to low voltage DC conversion as efficient as possible. Notably, the example versions shown in FIGS. 70-71 can be designed to be either isolated or non-isolated depending on the specifics of the end application and regulatory agency requirements. For example galvanic isolation can be realized if the AC to DC conversion block in FIG. 70 is isolated via a flyback or forward converter (or even a bulky 50/60 Hz transformer). In some embodiments the buck converter(s) (which could also be a buck-boost, boost-buck, boost, etc.) could be replaced with isolated flyback, forward converters, push-pull, half bridge, full bridge, etc. combinations of these. In addition to a galvanic isolated AC to DC conversion, the DC-DC conversion block could also employ a high-switching-frequency galvanic isolated transformer to achieve both efficiency and small size. As shown in the circuit 896 of FIG. 70, an AC input 898 is provided to an AC to DC conversion circuit 900, and high voltage DC power is provided via a high voltage DC bus 902 to one or more DC to DC buck converter(s) (or, again, buck-boost, boost-buck, boost, etc.) 904, and the resulting relatively low voltage DC is provided to load(s) via a low voltage bus 906. Although a buck converter is mentioned, any type of converter, including, but not limited to, buck, boost, boost-buck, buck-boost, Cuk, SEPIC, flyback, forward-converter, fly-back converter, etc. may be used. As shown in the circuit 908 of FIG. 71, an AC input 910 is provided to an AC to DC conversion circuit 912, and high voltage DC power is provided via a high voltage DC bus 914 to one or more DC to DC voltage down converters 916, and, for example, the resulting relatively low voltage DC is provided to load(s) via a low voltage bus 918. In other embodiments, higher output voltages may be used for the bus voltage. With regards to FIG. 71, it can be either isolated or non-isolated.

Turning to FIG. 72, an example embodiment of a solid state lighting system 920 is depicted in which AC power from an AC line input 922 is converted to DC in an AC to DC driver/power supply 924, and an N-channel driver 926 controls and monitors the DC current to multiple individual OLED panels, stacked OLED panels, RGB/RYB etc., OLED panels 928. The wired and/or wireless individual control feature can be considered optional in certain applications where wireless/wired control is not needed or lowest cost is paramount. The N channel individual control/monitor driver 926 (where N>1) can be of any practical number such as 12 individual channel control/monitoring drivers (typically N=2 to 256 with N greater than 1000 or higher also possible).

Various embodiments of the present invention can include smart and intelligent power supplies for OLEDs and OLED panels that are fixed color (e.g., white, blue or amber), color changing, white changing, blue and amber, etc. and special purpose OLEDs for medical, cleanroom, office, industrial, warehouse, stores, markets, grocery stores, subways, trains, subway stations, light rail stations, train stations, airports, bus stations, architecture design, etc., combinations of these, etc.

Implementations and embodiments include being able to accept a large range of input voltage requirements, output power/current and number of individual independent channels needed for the various types of OLED panels including white and other fixed color, white-changing, color-changing and multi-color, multi-panel, etc. combinations of these, etc.

Additional methods of intuitively and easily interacting with implementations and embodiments of the present invention include but are not limited to voice recognition and/or gesturing command control for the power/supplies, forward/reverse dimmer, and on/off elements of the present invention. In certain implementations of the present invention voice recognition control can be modular, field retrofit-compatible and installable, can seamlessly be integrated with the existing controls and can be readily adapted to and interfaced with the OLED power supplies/drivers.

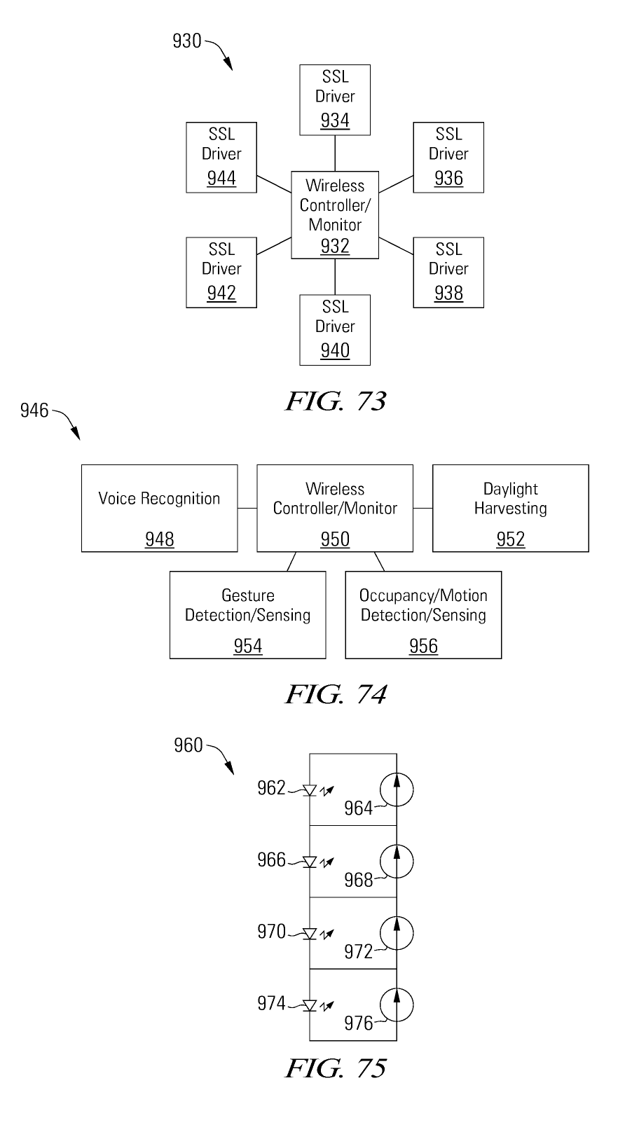

Turning to FIG. 73, a solid state lighting system 930 is depicted with a wireless controller/monitor 932 addressing and controlling six wireless OLED drivers 934, 936, 938, 940, 942, 944 which, in turn, each could be addressing N1, N2, N3 . . . , N6 OLED panels or, for example OLED colors where Ni (i=1 to 6) could all be the same or different. The wireless controller/monitor can be interfaced to, for example, an intranet, the Internet, custom remote controls, autonomous controls, Bluetooth, etc. and can be securely encrypted or unsecure.

Turning to FIG. 74, a solid state lighting system 946 is depicted with an example of the ways a wireless controller/monitor 950 can control, monitor and interact with ambient and human sensing, detection, recognition, information, etc., for example supporting voice recognition 948, gesture detection/sensing 954, occupancy/motion detection/sensing 956, daylight harvesting 952 through solar power collection, etc. In addition open source protocols can be used.

Implementations of the smart/intelligent power supplies/drivers for white-changing and color-changing OLEDs can also be color-changing LED power supply/driver products such that they are compatible and use the same interfaces and devices (i.e., smart phones, tablets, computers, laptops, etc.) and be managed/controlled/monitored with the same interfaces, protocols, etc. OLED-based applications include both full and partial color-changing lights that can also be essentially any or mostly any ‘shade’ (color temperature) of white light (i.e., dual/multi-mode applications) including desk/task lamps, can lights, table lamps, wall, floor, ceiling, down lights, track lights, sconce lights, under-cabinet, desk, table, other portable lighting, sconces, etc.

White-changing OLED panels can also provide a certain subset of color changing/tunability. The white-changing/tunable OLED panels and associated power supplies and drivers include, but are not limited to, under-cabinet and over cabinet lighting for kitchens, bathrooms, etc., desk, table, task, reading, and portable lamps/lights, accent lamp/lights and special environment lighting. An example of multiple floating output current control to drive white-changing and color changing panels is depicted in the schematic illustration 960 of FIG. 75, in which each SSL 962, 966, 970, 974 in a stack can be individually controlled by a corresponding constant current source 964, 968, 972, 976, respectively. Each constant current source (which can be, for example, remotely controlled/dimmed is configured to flow only into the corresponding OLED subpanel with no current from one current source flowing into a different subpanel/stack layer. The floating approach can also be used with blue and amber panels including stacked, integrated, etc. blue and amber OLED panels.

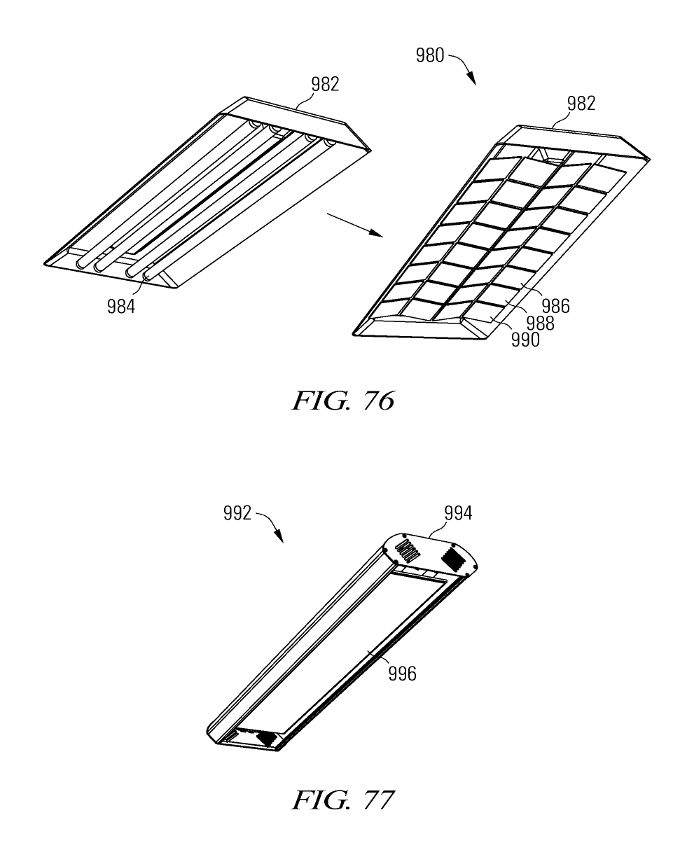

Turning now to FIG. 76, a retrofit 980 of a fluorescent lighting fixture 982 with an array of solid state lighting panels (e.g., 986, 988, 990) is depicted in accordance with some embodiments of the invention, in which fluorescent tubes (e.g., 984) are replaced by the array of solid state lighting panels (e.g., 986, 988, 990), along with a power controller/monitor and optional wired/wireless/other interfaces. In some embodiments, as in the illustration 992 of FIG. 77, a single SSL panel 996 can be mounted in a fixture 994, including existing fluorescent lamp fixtures. Such a SSL panel could be made/consist of LED, OLED, QD, other SSL, etc., combinations of these, etc.

Although an OLED panel is shown in FIGS. 61 and other figures including for example FIGS. 69-72, it can in general be any type of lighting panel including but not limited to SSL panels such as LED, OLED, QD, other SSL, combinations of these, etc.

The solid state lighting systems disclosed herein provide energy efficient, controllable and highly efficient lighting for a variety of applications. The lamps, lights, light fixtures, ballasts, desk lamps, etc., can have IR LED modules or arrays mounted or attached to them. These lamps, lights, light fixtures, ballasts, desk lamps, etc., can receive commands from the user that were outputted to IR devices in order to control them by turning them on/off, etc. Attaching the IR LEDs to the lamps, lights, light fixtures, ballasts, desk lamps, etc., can often give them a better point of view and access to the devices to be controlled in the room or building. For example, IR LEDs can be mounted into a screw in socket type light bulb fixture or any other type of mounted fixture with the LEDs pointing so as to surround the light, making it easier to transmit the IR to all areas of a room or building. These IR LEDs can also be installed into a ballast type light fixture. This has the same or similar effects as the previously mentioned IR array but in different types of configuration in the fixture (i.e., linear, angled, tilted, etc.). These IR LEDs can also be installed into a lamp or fixture that has servos, stepper motors, or other motors, which can allow the light or fixture to move. This allows the IR LEDs to be mobile as well in the case that the IR device is out of view from the LEDs. IR LEDs can be placed on the tops, sides, bottoms, etc., of the light fixtures to better send IR commands to their corresponding devices in the room. This helps transmission of information to televisions, heaters, air-conditioners, fans, etc. The IR LEDs are not for lighting purposes but for sending commands to IR controlled devices specifically.

The present invention uses LEDs and OLEDs which are energy efficient and use less energy in the long term compared to conventional lighting types. These lighting types can benefit hospitals, schools, libraries, convalescent homes, assisted living homes, colleges, universities, dormitories, etc.

In conclusion, embodiments of the present invention provide novel systems, devices, methods and arrangements for solar energy collection. While detailed descriptions of one or more embodiments of the invention have been given above, various alternatives, modifications, and equivalents will be apparent to those skilled in the art without varying from the spirit of the invention. Therefore, the above description should not be taken as limiting the scope of embodiments of the invention which are encompassed by the appended claims.

What is claimed is:

1. A solid state lighting system, comprising:
at least one solid state light;
a power supply powering the at least one solid state light; and
a control circuit operable to control an electrical current to the at least one solid state light and to cause the at least one solid state light to change color based on time of day.

2. The solid state lighting system of claim 1, wherein the at least one solid state light comprises at least one organic light emitting diode panel.

3. The solid state lighting system of claim 2, wherein the at least one organic light emitting diode panel comprises multiple panels.

4. The solid state lighting system of claim 3, wherein the control circuit comprises a separate control channel for each of the multiple panels.

5. The solid state lighting system of claim 2, further comprising a solid state point light source.

6. The solid state lighting system of claim 5, wherein the solid state point light source comprises a light emitting diode.

7. The solid state lighting system of claim 5, wherein the solid state point light source comprises a quantum dot.

8. The solid state lighting system of claim 1, wherein the color change is programmed based on circadian rhythms.

9. The solid state lighting system of claim 1, further comprising a threaded lamp base operable to screw into a lamp socket.

10. The solid state lighting system of claim 1, further comprising a connector operable to connect to a fluorescent lamp fixture.

11. The solid state lighting system of claim 1, further comprising at least one IR sensor operable to receive remote control commands from at least one remote device.

12. The solid state lighting system of claim 11, further comprising at least one IR transmitter operable to forward the remote control commands.

13. The solid state lighting system of claim 1, wherein the at least one solid state light comprises a plurality of lights, and wherein the control circuit comprises a channel for each of the plurality of lights.

14. The solid state lighting system of claim 13, wherein the control circuit comprises floating output current control.

15. The solid state lighting system of claim 1, wherein the power supply comprises a constant current power supply.

16. The solid state lighting system of claim 1, wherein the power supply comprises a linear regulator and a switching regulator.

17. The solid state lighting system of claim 16, wherein either the linear regulator and the switching regulator is selected based on a dimming level of the solid state lighting system.

18. The solid state lighting system of claim 1, wherein the at least one solid state light is rotatably mounted to a base.

19. The solid state lighting system of claim 18, further comprising at least one motor operable to position the at least one solid state light.

20. The solid state lighting system of claim 19, wherein the control circuit is operable to control the at least one motor.
